(19) United States
(12) Patent Application Publication
(10) Pub. No.: US 2024/0270786 A1
(43) Pub. Date: Aug. 15, 2024

(54) STEROID COMPOUNDS

(71) Applicant: Epirium Bio Inc., San Diego, CA (US)

(72) Inventors: Sundeep DUGAR, San Jose, CA (US); Bhavesh DAVE, Maharashtra (IN); Ajay Ramchandra TILEKAR, Maharashtra (IN); Somdutta SEN, Manesar (IN)

(21) Appl. No.: 18/391,191

(22) Filed: Dec. 20, 2023

Related U.S. Application Data
(60) Provisional application No. 63/434,417, filed on Dec. 21, 2022.

(51) Int. Cl. C07J 73/00; C07J 63/00

(52) U.S. Cl. CPC C07J 73/005; C07J 63/002

(57) ABSTRACT

The present application discloses derivatives of steroids, pharmaceutically acceptable salts, stereoisomers or tautomers thereof, and processes for preparation thereof. The compounds and compositions described herein can be used in therapy.

### FIELD

Disclosed herein are novel compounds of 11β-hydroxy steroids and related compounds and compositions and their application as pharmaceuticals for preventing or reversing injury to mitochondria, for treating or preventing diseases relating to mitochondrial dysfunction or depletion, and for inducing regeneration or restructuring of mitochondria as a means of treating diseases relating to abnormalities in mitochondrial structure and function in a human or animal subject. Also disclosed herein are methods for diagnosing injury to mitochondria and for diagnosing the success or failure of therapeutics designed to treat, prevent, or reverse injury to or depletion of mitochondria.

### BACKGROUND

Mitochondria are responsible for generating more than 90% of the energy needed by the body to sustain life and support growth. When mitochondrial function fails, less energy is generated within the cell, resulting in cell injury and ultimately cell death. Mitochondria are susceptible to degradation due to oxygen radicals produced by their own metabolic processes. Damaged mitochondria involute and are expelled by the cell. Their replacement by new mitochondria is called mitochondrial biogenesis. The proliferation of mitochondria or their hypertrophy to meet increased metabolic demand is also called mitochondrial biogenesis. It is signified by the expression of additional mitochondrial proteins, particularly those related to oxidative phosphorylation. The capacity for mitochondrial biogenesis is significantly lost with age. Thus many diseases of aging are associated with loss of mitochondria in various tissues, whose specialized function is diminished in the context of diminished mitochondrial function and/or number. Many disease states, such as those that have neuromuscular disease symptoms, sarcopenia, muscular dystrophy, diabetes mellitus, dementia, Parkinson's disease, Huntington's disease, amyotrophic lateral sclerosis (ALS), obesity, hyperlipidemia, heart failure, lupus, and ocular conditions such as age-related macular degeneration (AMD), are associated with progressive mitochondrial loss in various tissues.

In addition, a number of drugs and drug classes also have an effect on mitochondrial function and biogenesis and can affect organ function and even lead to organ degeneration or other side effects which are directly related to the toxic effect of these drugs on the mitochondria.

A non-limiting list of drugs and drug classes that are associated with their mitochondria effect can be found in: Pereira et al., Current Drug Safety, 4:34-54, 2009; Gohil et al., Nature Biotechnol., 28:249-257, 2010; and Wagner et al., Nature Biotechnol., 26:343-351, 2008, each of which is hereby incorporated by reference in its entirety. Reflecting this understanding, the phrase “mitochondrial toxicity” as used herein refers to failure of the mitochondria resulting from the administration of chemical compositions to a subject.

Ischemic and ischemia/reperfusion injury are accompanied by decreases in mitochondrial function and number, leading to apoptotic cell death, necrosis, and functional organ deterioration in ischemic conditions such as myocardial infarction and stroke. Despite considerable advances in the diagnosis and treatment of such conditions, there remains a need for prophylactic and therapeutic approaches for the treatment of these conditions.

Mitochondria are critical to cell function and the effects of mitochondrial disease can be varied and can take on unique characteristics. The severity of the specific defect may be great or small and often affect the operation of the mitochondria and multiple tissues more severely, leading to multi-system diseases. Injury to, or dysfunction of, skeletal muscle mitochondria generally results in muscle weakness and atrophy, termed sarcopenia in severe states. In the case of generalized muscle weakness, reduction in bone density can be generalized, one of the causes of the bone disease known as osteoporosis. Depleted mitochondria in the heart can eventuate in the symptoms of congestive heart failure and eventual death. Loss of mitochondrial density in the brain is associated with neurodegeneration states such as Huntington's disease, Alzheimer's disease, and Parkinson's disease. Generalized loss of mitochondria including liver mitochondria can result in hyperlipidemia, hypertension, and insulin resistance progression to Type 2 diabetes. Liver mitochondria are injured by fructose uptake. Fructose, uric acid, and other agents injurious to liver mitochondria can cause accumulation of intracellular lipids, particularly triglycerides that contribute to the syndrome of hepatic steatosis, and increased synthesis and export of triglycerides that contributes to systemic hyperlipidemia, and ultimately obesity and insulin resistance.

Treatment options are currently limited and there remains a need for prophylactic and therapeutic approaches for the treatment of these conditions associated with chronic mitochondrial dysfunction and toxicity. Thus there is a need for treatments that stimulate mitochondrial function in response to increased metabolic demand and induce mitochondrial replication in response to agents or conditions that cause depletion of mitochondria in one or more tissues. Complicating potential therapies is the fact that the aging process is generally associated with progressive loss of the ability to support mitochondrial biogenesis, for reasons that are unknown.

### SUMMARY

Provided herein are compounds, salts thereof, pharmaceutical compositions of the foregoing and methods of making and using the same. In one aspect, provided is a compound of formula (I):

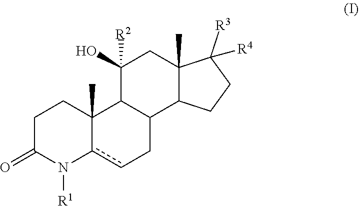

or a pharmaceutically acceptable salt thereof, wherein R¹, R², R³, and R⁴ are as detailed herein.

In one aspect, provided is a compound of formula (II):

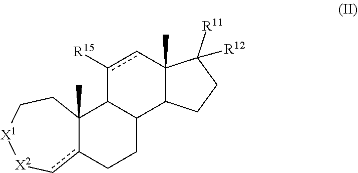

or a pharmaceutically acceptable salt thereof, wherein X¹, X², R¹¹, R¹², and R¹⁵ are as detailed herein.

In one aspect, provided is a compound of formula (III):

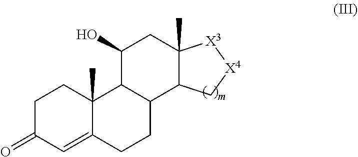

or a pharmaceutically acceptable salt thereof, wherein X³, X⁴, X⁵, R¹⁶, R¹⁷, and m are as detailed herein.

Also provided is a pharmaceutical composition comprising a compound of any formula herein, including formulas (I) or (II), a pharmaceutically acceptable salt thereof, and a pharmaceutically acceptable carrier.

Hydroxysteroids are hydroxylated compounds with a sterol structure and are are known to be produced in cells when the mitochondria are exposed to high levels of endogenous H₂O₂ which then acts via the mitochondrial enzyme, 11β-hydroxylase, to hydroxylate a variety of steroids, including cholesterol, pregnenolone, progesterone, and others. Hydroxylation can occur in numerous positions, including the 7, 16, and 11 positions. These molecules, termed hydroxysteroids, are then sulfated and secreted into the extracellular space, where in the brain they modulate GABA-receptors and calcium channels on the plasma membrane. No intracellular activity of hydroxysteroids has previously been described.

### BRIEF DESCRIPTION OF DRAWINGS

FIG. 1. ATP synthesis and hydrolysis of compounds 26, 40, and (+)-epicatechin in fresh heart mitochondria.

FIG. 2. Oligomycin controls of ATP synthesis and hydrolysis in fresh heart mitochondria.

FIG. 3. ATP hydrolysis assay of 26, 40, and (+)-epicatechin in frozen heart mitochondria.

### DETAILED DESCRIPTION

The present application discloses derivatives of steroids, pharmaceutically acceptable salts, stereoisomers or tautomers thereof, and processes for preparation thereof. The compounds and compositions described herein can be used in therapy.

### Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group can be referred to as a “perhaloalkyl.” An exemplary perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to a subject (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

“Pharmaceutically acceptable salts” are those salts which retain at least some of the biological activity of the free (non-salt) compound and which can be administered as drugs or pharmaceuticals to a subject. Such salts, for example, include, without limitation: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound in its free acid or base form with a suitable organic or inorganic base or acid, respectively, and isolating the salt thus formed during subsequent purification.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

### Compounds

In some embodiments, provided is a compound of formula (I),

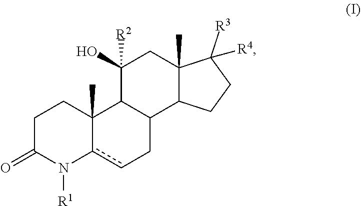

or a pharmaceutically acceptable salt, stereoisomer or tautomer thereof, wherein:
R¹ is H or C₁₋₆ alkyl;
R² is H, D, or C₁₋₆ alkyl;
R³ is C₁₋₆ alkyl, C₁₋₆ alkoxy, C(O)R⁵, NC(O)R⁵, NC(O)NR₆R₇, C(O)OR⁵, NHR⁵, or 5-membered heterocyclyl, wherein C₁₋₆ alkyl is optionally substituted with one or more of OH or D, and 5-membered heterocyclyl is optionally substituted with C₁₋₆ alkyl; R⁴ is H;
or R³ and R⁴ taken together with the carbon atom to which they are attached form a 5-membered heterocycle;
R⁵ is H, C₁₋₆ alkyl, C₃₋₁₀ cycloalkyl, SO₂R⁶, NR⁶R⁷ or 5-6 membered heterocyclyl optionally substituted with one or more C₁₋₆ alkyl;
R⁶ and R⁷ are each independently H, C₃₋₆ cycloalkyl, or C₁₋₆ alkyl optionally substituted with C₃₋₆ cycloalkyl; and
the dashed line represents an optional double bond.

In some embodiments, R¹ is H. In some embodiments, R¹ is methyl.

In some embodiments, R² is H. In some embodiments, R² is D. In some embodiments, R² is methyl.

In some embodiments, R³ is C₁₋₆ alkyl substituted with OH. In some embodiments, R³ is ethyl substituted with OH. In some embodiments, R³ is ethyl substituted with OH and D. In some embodiments, R³ is ethyl substituted with OH and methyl. In some embodiments, R³ is methoxy. In some embodiments, R³ is ethoxy. In some embodiments, R³ is NH₂. In some embodiments, R³ is acetyl. In some embodiments, R³ is

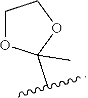 [SMILES: *C1(C)OCCO1]

In some embodiments, R³ is C(O)R⁵. In some embodiments, R³ is C(O)OR⁵. In some embodiments, R³ is NC(O)R⁵.

In some embodiments, R⁵ is methyl. In some embodiments, R⁵ is ethyl. In some embodiments, R⁵ is isopropyl. In some embodiments, R⁵ is cyclopropyl. In some embodiments, R⁵ is cyclobutyl. In some embodiments, R⁵ is OH. In some embodiments, R⁵ is methoxy. In some embodiments, R⁵ is NH₂. In some embodiments, R⁵ is selected from the group consisting of

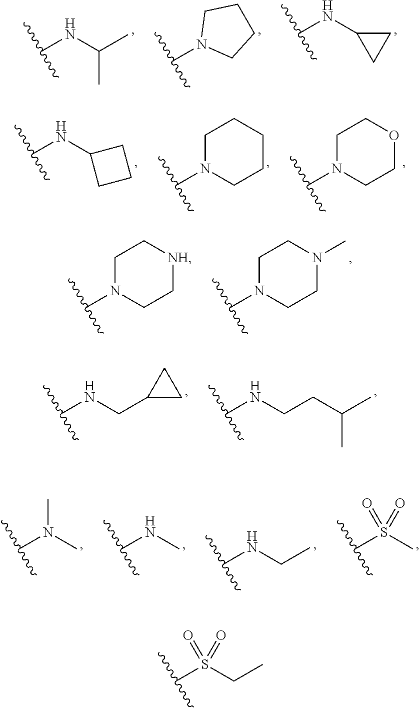

In some embodiments, R³ and R⁴ taken together with the carbon atom to which they are attached form

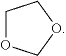

In some embodiments, R¹ is methyl, and the dashed line is a double bond. In some embodiments, R¹ is methyl, and R² is hydrogen. In some embodiments, R¹ is methyl, the dashed line is a double bond, and R² is hydrogen. In some embodiments, R¹ is methyl, the dashed line is a double bond, and R³ is C(O)R⁵, NC(O)R⁵, NC(O)NR₆R₇, or C(O)OR⁵.

In some embodiments, provided is a compound of formula (Ia),

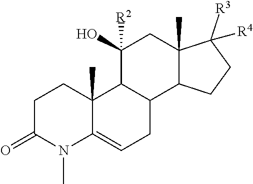

or a pharmaceutically acceptable salt, stereoisomer or tautomer thereof, wherein each variable is as defined for formula (I) or any variation thereof.

In some embodiments, provided is a compound of formula (Ib),

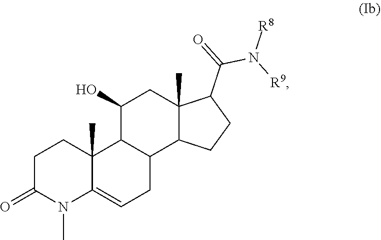

or a pharmaceutically acceptable salt, stereoisomer or tautomer thereof, wherein R⁸ and R⁹ are each independently H, C₃₋₆ cycloalkyl, or C₁₋₆ alkyl optionally substituted with C₃₋₆ cycloalkyl; or
R⁸ and R⁹ together with the nitrogen atom to which they are attached form a 5- or 6-membered heterocycle; and
each remaining variable is as defined for formula (I) or any variation thereof.

In some embodiments, R⁸ is methyl. In some embodiments, R⁸ is isopropyl. In some embodiments, R⁸ is cyclopropyl. In some embodiments, R⁸ is methyl substituted with cyclopropyl. In some embodiments, R⁸ is cyclobutyl. In some embodiments, R⁹ is methyl. In some embodiments, R⁹ is hydrogen. In some embodiments, R⁸ is

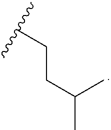 [SMILES: *CCC(C)C]

In some embodiments, R⁸ and R⁹ together with the nitrogen atom to which they are attached form a ring selected from the group consisting of

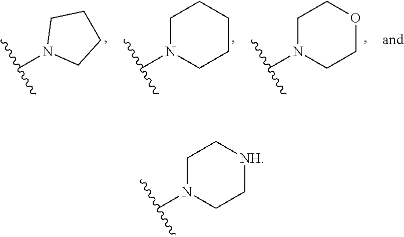

In some embodiments, provided is a compound of formula (Ic),

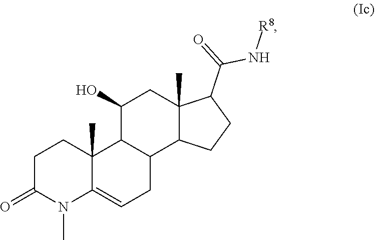

or a pharmaceutically acceptable salt, stereoisomer or tautomer thereof, wherein each variable is as defined for formula (I) or any variation thereof.

In some embodiments, R⁸ is hydrogen. In some embodiments, R⁸ is C₁₋₆ alkyl. In some embodiments, R⁸ is methyl. In some embodiments, R⁸ is isopropyl. In some embodiments, R⁸ is C₃₋₁₀ cycloalkyl. In some embodiments, R⁸ is cyclopropyl. In some embodiments, R⁸ is cyclobutyl. In some embodiments, R⁸ is C₁₋₆ alkyl substituted with C₃₋₁₀ cycloalkyl. In some embodiments, R⁸ is methyl substituted with cyclopropyl. In some embodiments, R⁸ is

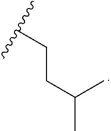 [SMILES: *CCC(C)C]

In some embodiments, provided is a compound of formula (Id),

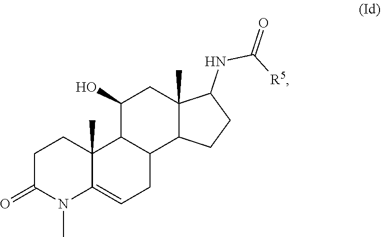

or a pharmaceutically acceptable salt, stereoisomer or tautomer thereof, wherein each variable is as defined for formula (I) or any variation thereof.

In some embodiments, R⁵ is C₁₋₆ alkyl. In some embodiments, R⁵ is methyl. In some embodiments, R⁵ is ethyl. In some embodiments, R⁵ is isopropyl. In some embodiments, R⁵ is C₃₋₁₀ cycloalkyl. In some embodiments, R⁵ is cyclopropyl. In some embodiments, R⁵ is cyclobutyl.

In some embodiments, R⁵ is 6-membered heterocyclyl. In some embodiments, R⁵ is 6-membered heterocyclyl optionally substituted with one or more C₁₋₆ alkyl. In some embodiments, R⁵ is 6-membered heterocyclyl optionally substituted with methyl. In some embodiments, R⁵ is selected from the group consisting of

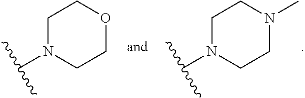

In some embodiments, provided is a compound of formula (Ie),

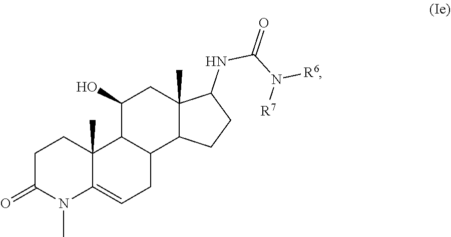

or a pharmaceutically acceptable salt, stereoisomer or tautomer thereof, wherein each variable is as defined for formula (I) or any variation thereof.

In some embodiments, R⁶ is C₁₋₆ alkyl. In some embodiments, R⁶ is ethyl. In some embodiments, R⁶ is C₃₋₁₀ cycloalkyl. In some embodiments, R⁶ is cyclobutyl. In some embodiments, R⁶ is C₁₋₆ alkyl optionally substituted with C₃₋₁₀ cycloalkyl.

In some embodiments, the compound of Formula (I) is selected from the group consisting of:

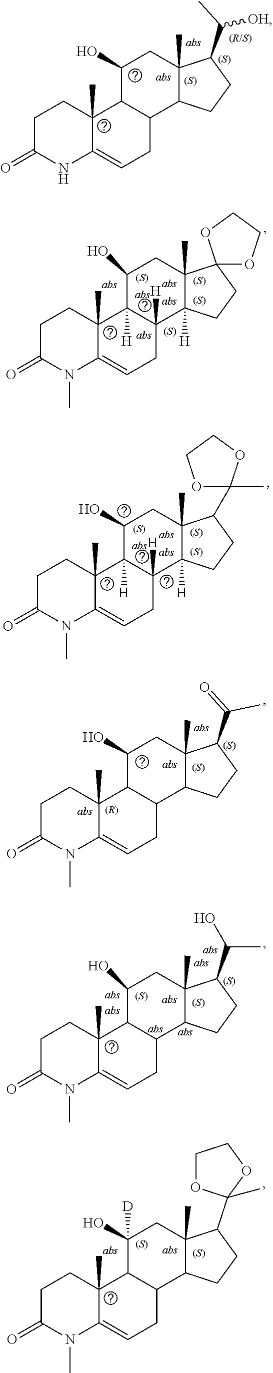

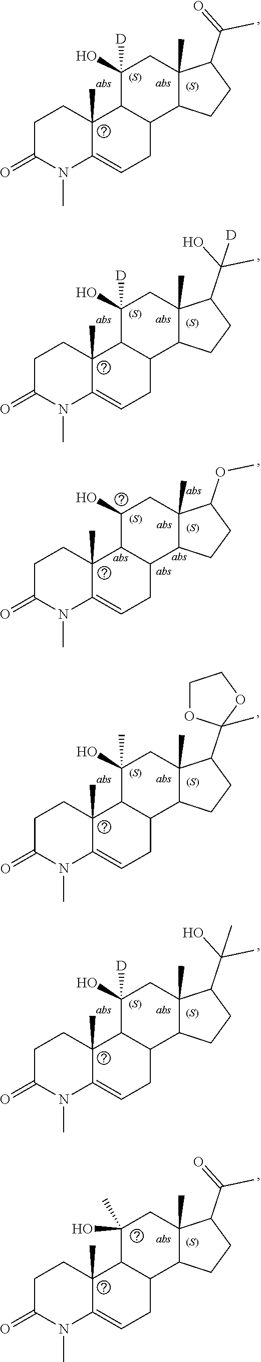

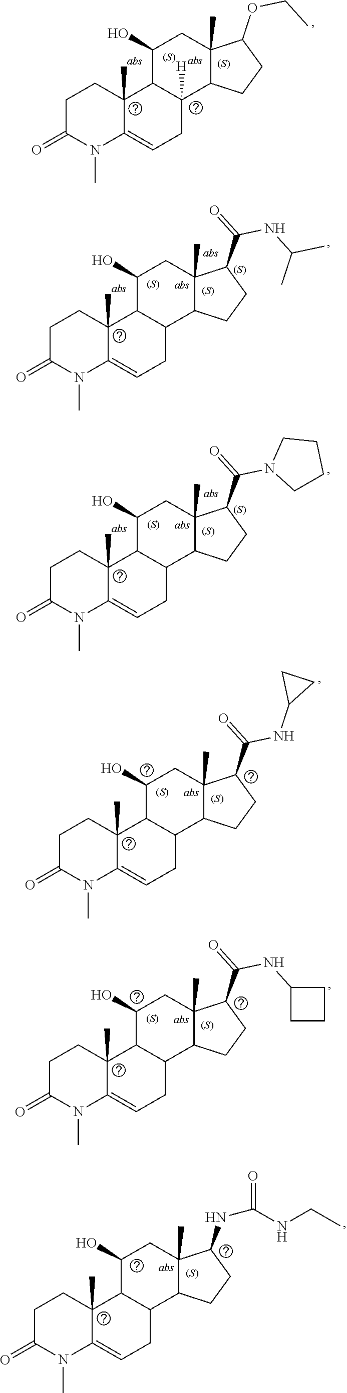

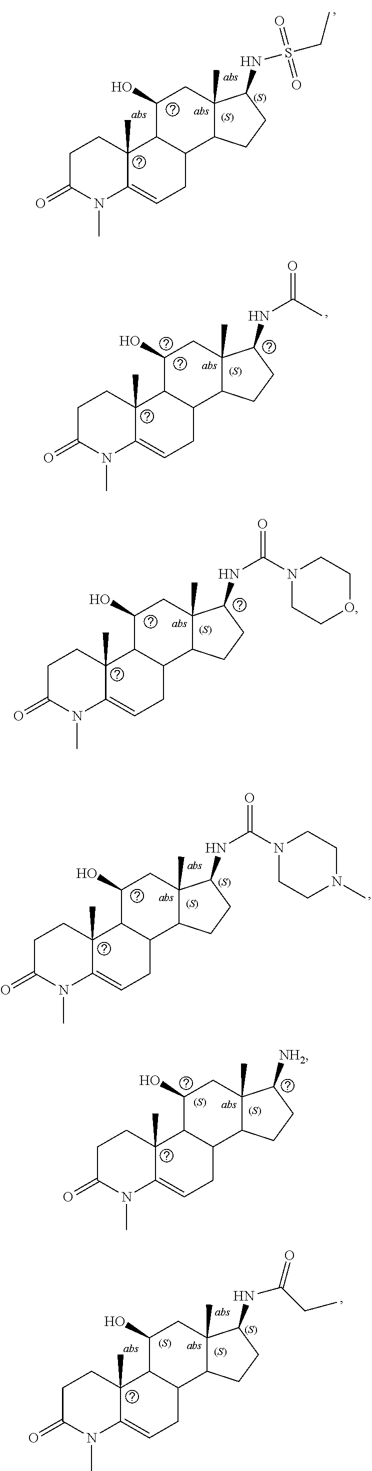

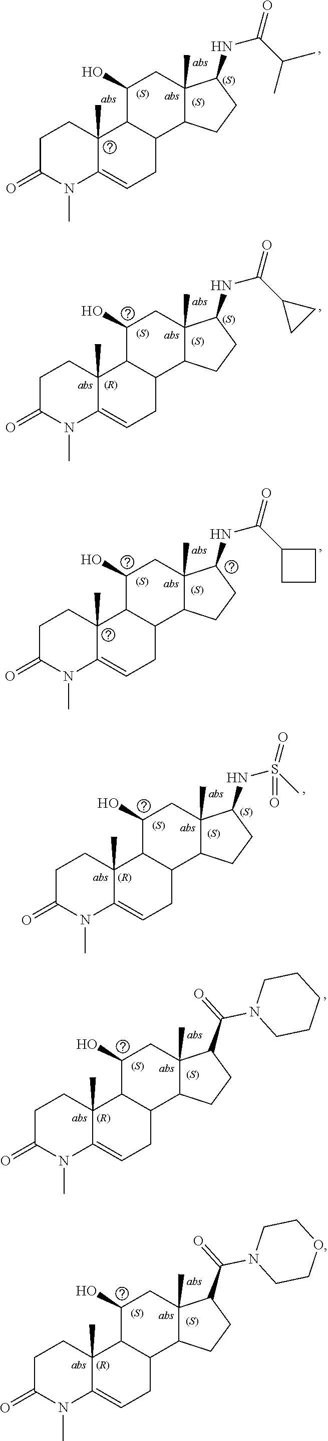

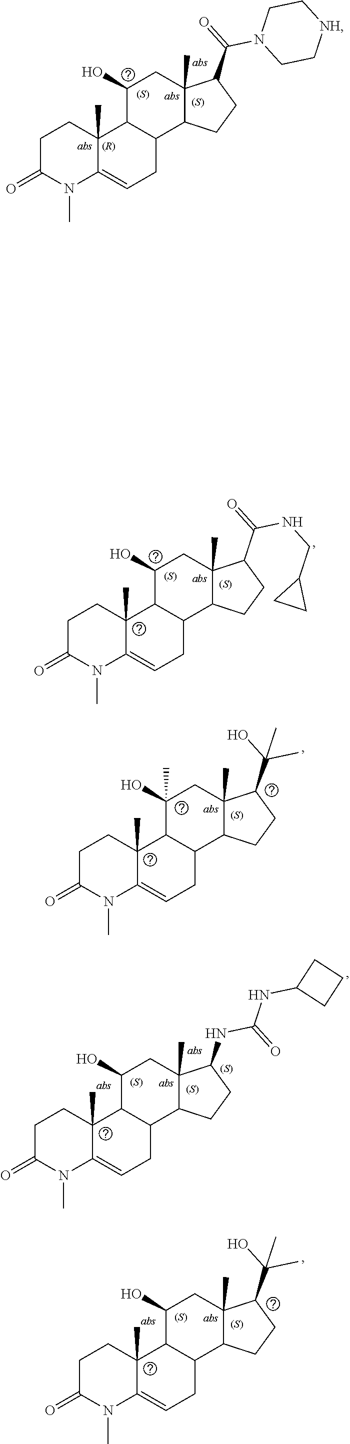

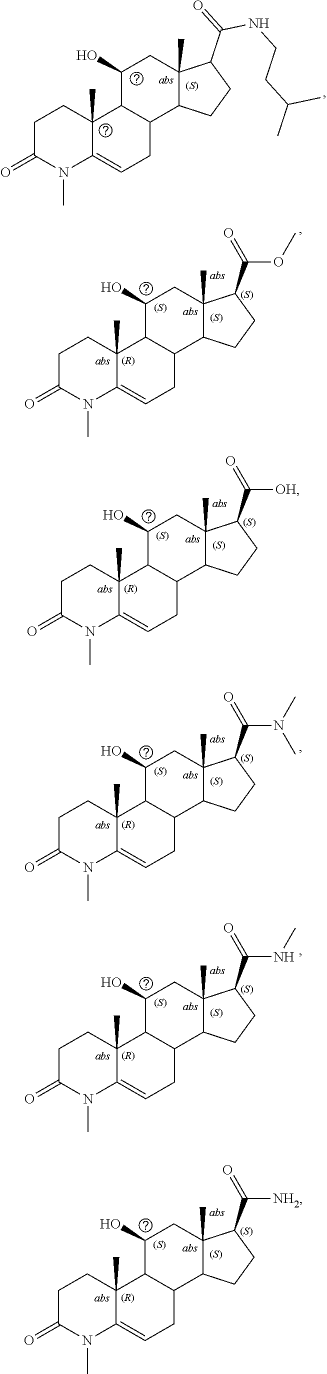

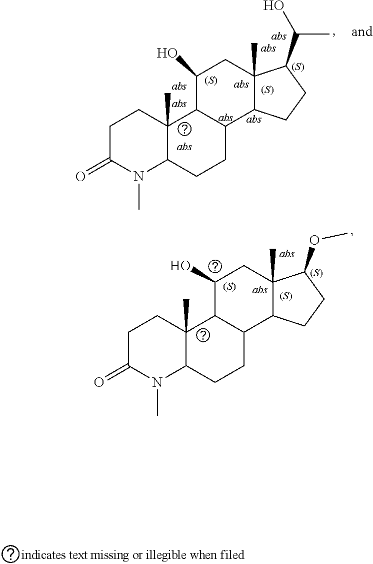

or a pharmaceutically acceptable salt, stereoisomer or tautomer thereof.

In some aspects, provided is a compound of formula (II),

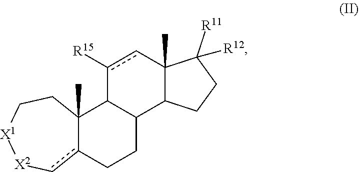

wherein:
one of X¹ and X² is NR¹⁰, and the other is C═O,
R¹⁰ is H or C₁₋₆ alkyl;
R¹¹ is C₁₋₆ alkoxy, C(O)OR¹³, C(O)NR¹³R¹⁴, C(O)R⁴, NR¹³R¹⁴, NC(O)R¹³, or C₁₋₆ alkyl optionally substituted with one or more R¹³;
R¹² is H or OH; or
R¹¹ and R¹² together form oxo;
R¹³ and R¹⁴ are each independently H, C₁₋₆ alkyl, C₃₋₆ cycloalkyl, or NH₂;
R¹⁵ is H or OH; and
   each dashed line represents an optional double bond.

In some embodiments, X¹ is NR¹⁰, and X² is C═O. In some embodiments, X¹ is C═O, and X² is NR¹⁰. In some embodiments, R¹⁰ is hydrogen. In some embodiments, R¹⁰ is or C₁₋₆ alkyl. In some embodiments, R¹⁰ is methyl.

In some embodiments, R¹¹ is C₁₋₆ alkoxy. In some embodiments, R¹¹ is methoxy. In some embodiments, R¹¹ is carboxylic acid. In some embodiments, R¹¹ is acetyl. In some embodiments, R¹¹ is NH₂. In some embodiments, R¹¹ is C₁₋₆ alkyl optionally substituted with one or more C₁₋₆ alkyl, C₃₋₆ cycloalkyl, or NH₂. In some embodiments, R¹¹ is ethyl. In some embodiments, R¹¹ is ethyl substituted with NH₂.

In some embodiments, R¹¹ is selected from the group consisting of

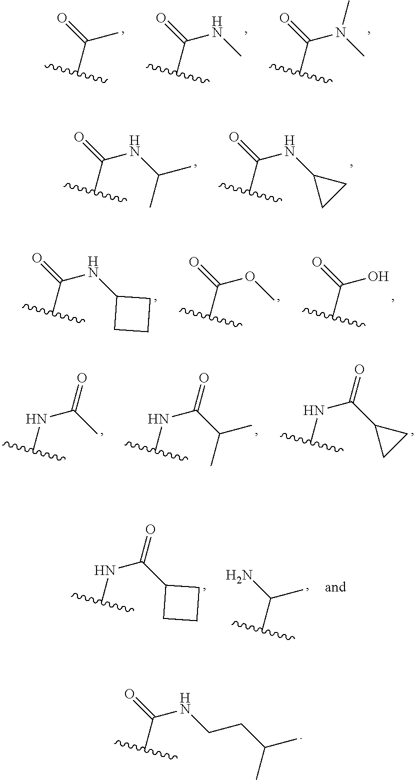

In some embodiments, R¹² is hydrogen. In some embodiments, R¹² is OH. In some embodiments, R¹¹ and R¹² together form oxo.

In some embodiments, R¹⁵ is H. In some embodiments, R⁵ is OH.

In some embodiments, X¹ is C═O, and X² is N—CH₃. In some embodiments, X¹ is C═O, X² is N—CH₃, and R¹¹ is C(O)OR¹³, C(O)NR¹³R¹⁴, or NC(O)R¹³.

In some embodiments, provided is a compound of formula (II-1),

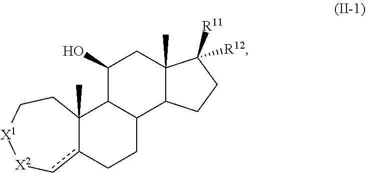

or a pharmaceutically acceptable salt, stereoisomer or tautomer thereof, wherein each variable is as defined for formula (II) or any variation thereof.

In some embodiments, provided is a compound of formula (II-2),

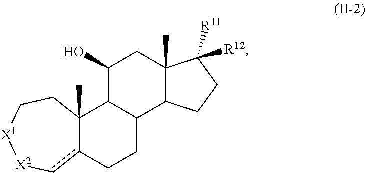

or a pharmaceutically acceptable salt, stereoisomer or tautomer thereof, wherein each variable is as defined for formula (II) or any variation thereof.

In some embodiments, provided is a compound of formula (IIa),

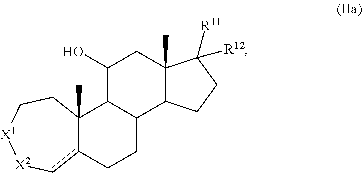

or a pharmaceutically acceptable salt, stereoisomer or tautomer thereof, wherein each variable is as defined for formula (II) or any variation thereof.

In some embodiments, X¹ is NR¹⁰, and X² is C═O. In some embodiments, X¹ is C═O, and X² is NR¹⁰. In some embodiments, R¹⁰ is hydrogen. In some embodiments, R¹⁰ is or C₁₋₆ alkyl. In some embodiments, R¹⁰ is methyl.

In some embodiments, provided is a compound of formula (IIb),

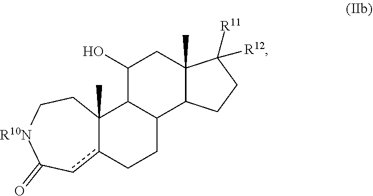

or a pharmaceutically acceptable salt, stereoisomer or tautomer thereof, wherein each variable is as defined for formula (II) or any variation thereof.

In some embodiments, R¹⁰ is or C₁₋₆ alkyl. In some embodiments, R¹⁰ is methyl. In some embodiments, R¹² is hydrogen. In some embodiments, R¹² is OH.

In some embodiments, provided is a compound of formula (IIc),

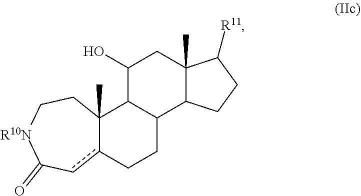

or a pharmaceutically acceptable salt, stereoisomer or tautomer thereof, wherein each variable is as defined for formula (II) or any variation thereof.

In some embodiments, R¹⁰ is C₁₋₆ alkyl. In some embodiments, R¹⁰ is methyl.

In some embodiments, R¹¹ is C₁₋₆ alkoxy. In some embodiments, R¹¹ is methoxy. In some embodiments, R¹¹ is carboxylic acid. In some embodiments, R¹¹ is acetyl. In some embodiments, R¹¹ is NH₂. In some embodiments, R¹¹ is C₁₋₆ alkyl optionally substituted with one or more C₁₋₆ alkyl, C₃₋₆ cycloalkyl, or NH₂. In some embodiments, R¹¹ is ethyl. In some embodiments, R¹¹ is ethyl substituted with NH₂.

In some embodiments, R¹¹ is selected from the group consisting of

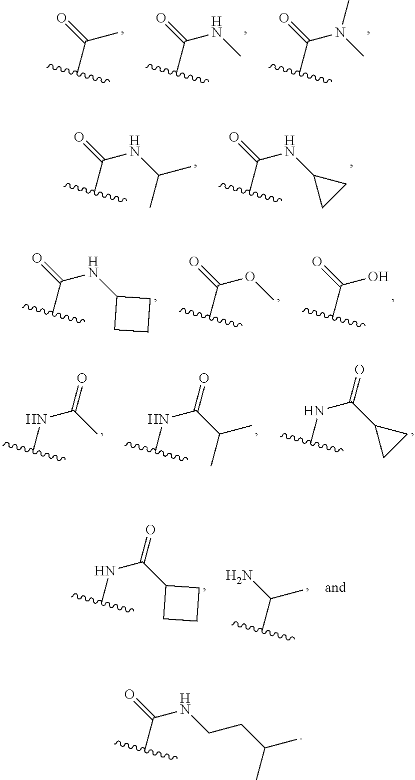

In some embodiments, provided is a compound of formula (IId),

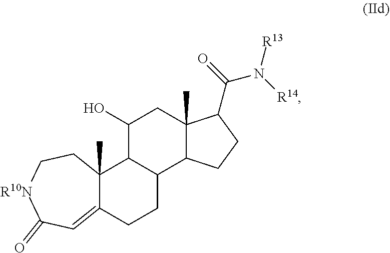

or a pharmaceutically acceptable salt, stereoisomer or tautomer thereof, wherein each variable is as defined for formula (II) or any variation thereof.

In some embodiments, R¹⁰ is C₁₋₆ alkyl. In some embodiments, R¹⁰ is methyl.

In some embodiments, R¹³ is hydrogen. In some embodiments, R¹³ is C₁₋₆ alkyl. In some embodiments, R¹³ is methyl. In some embodiments, R¹³ is isopropyl. In some embodiments, R¹³ is C₃₋₆ cycloalkyl. In some embodiments, R¹³ is cyclopropyl. In some embodiments, R¹³ is cyclobutyl. In some embodiments, R¹³ is

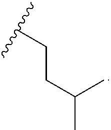 [SMILES: *CCC(C)C]

In some embodiments, R¹⁴ is hydrogen. In some embodiments, R¹⁴ is C₁₋₆ alkyl. In some embodiments, R¹⁴ is methyl.

In some embodiments, provided is a compound of formula (IIe),

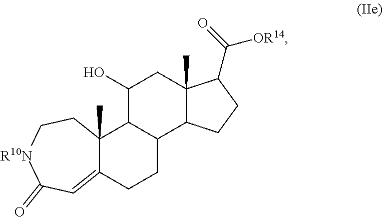

or a pharmaceutically acceptable salt, stereoisomer or tautomer thereof, wherein each variable is as defined for formula (II) or any variation thereof.

In some embodiments, R¹⁰ is C₁₋₆ alkyl. In some embodiments, R¹⁰ is methyl.

In some embodiments, R¹⁴ is hydrogen. In some embodiments, R¹⁴ is C₁₋₆ alkyl. In some embodiments, R¹⁴ is methyl.

In some embodiments, provided is a compound of formula (IIf),

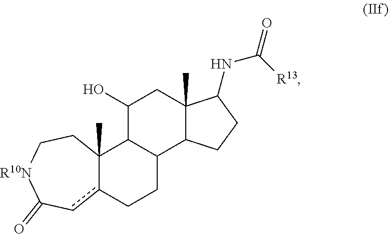

or a pharmaceutically acceptable salt, stereoisomer or tautomer thereof, wherein each variable is as defined for formula (II) or any variation thereof.

In some embodiments, R¹⁰ is C₁₋₆ alkyl. In some embodiments, R¹⁰ is methyl.

In some embodiments, R¹³ is C₁₋₆ alkyl. In some embodiments, R¹³ is methyl. In some embodiments, R¹³ is isopropyl. In some embodiments, R¹³ is C₃₋₆ cycloalkyl. In some embodiments, R¹³ is cyclopropyl. In some embodiments, R¹³ is cyclobutyl.

In some embodiments, provided is a compound of formula (IIg),

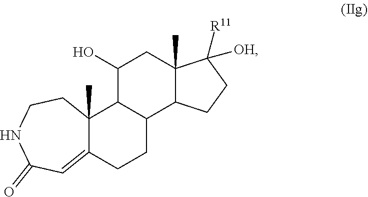

or a pharmaceutically acceptable salt, stereoisomer or tautomer thereof, wherein each variable is as defined for formula (II) or any variation thereof.

In some embodiments, R¹¹ is C(O)NHR¹³. In some embodiments, R¹³ is C₁₋₆ alkyl or C₃₋₆ cycloalkyl. In some embodiments, R¹³ is isopropyl or isopentyl. In some embodiments, R¹³ is cyclopropyl or cyclobutyl.

In some embodiments, R¹¹ is selected from the group consisting of

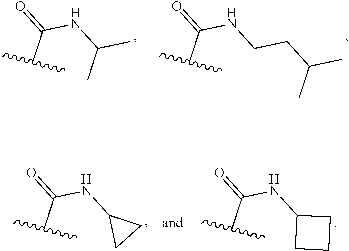

In some embodiments, provided is a compound of formula (IIh),

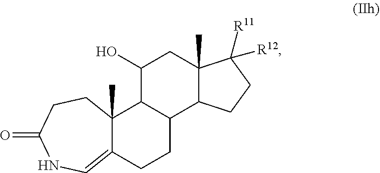

or a pharmaceutically acceptable salt, stereoisomer or tautomer thereof, wherein each variable is as defined for formula (II) or any variation thereof.

In some embodiments, R¹¹ is selected from the group consisting of

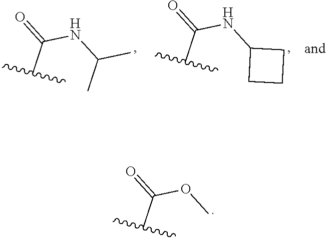

In some embodiments, R¹² is hydrogen.

In some embodiments, the compound of formula (II) is selected from the group consisting of:

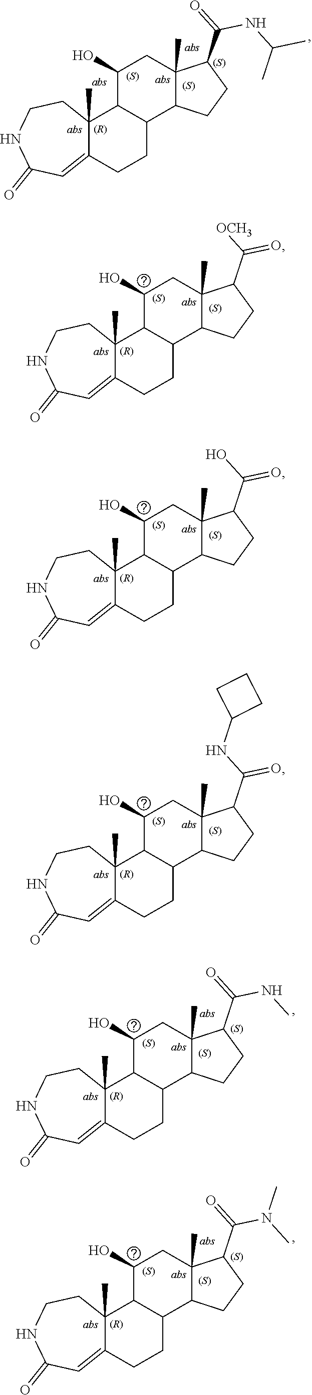

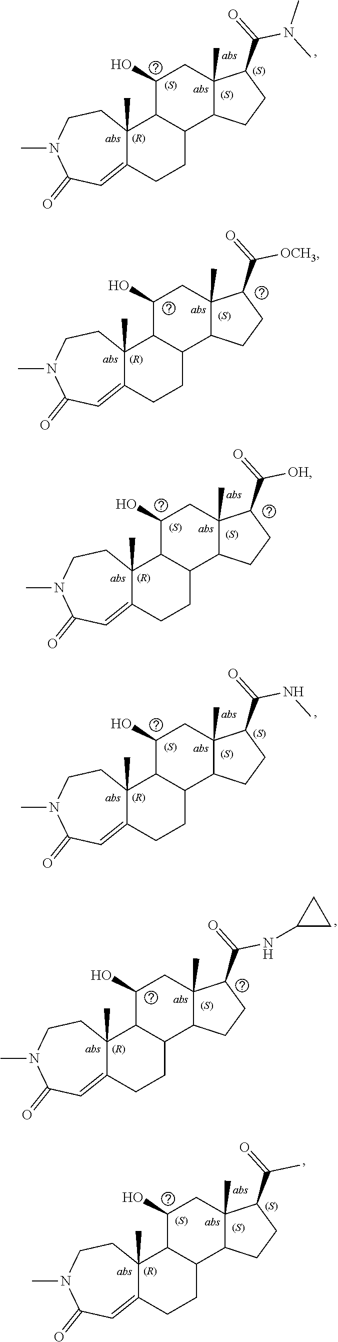

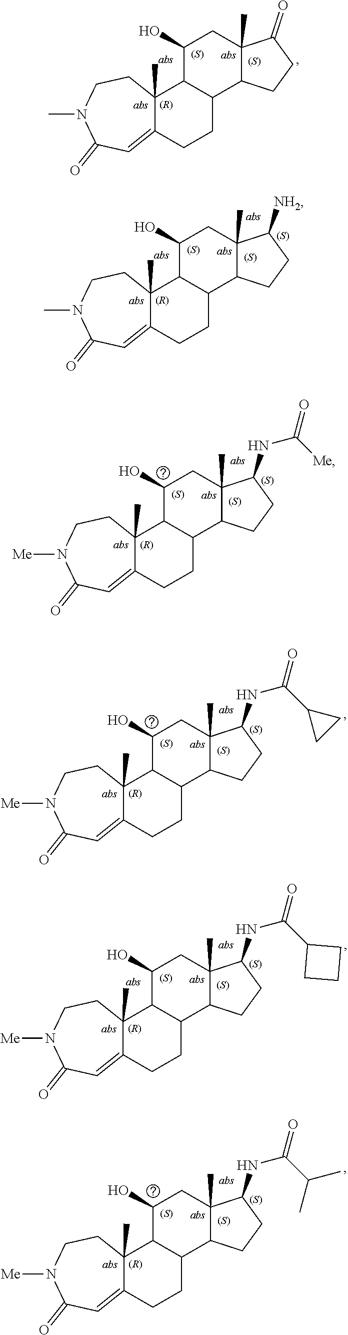

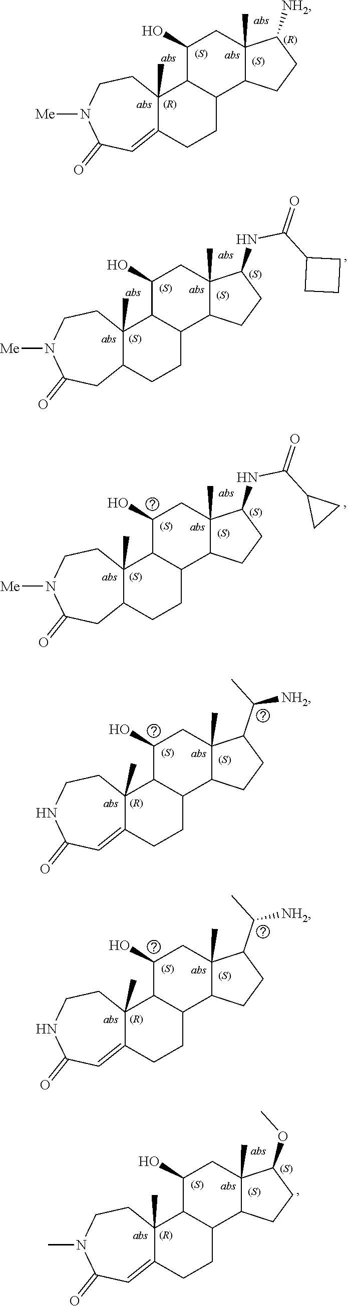

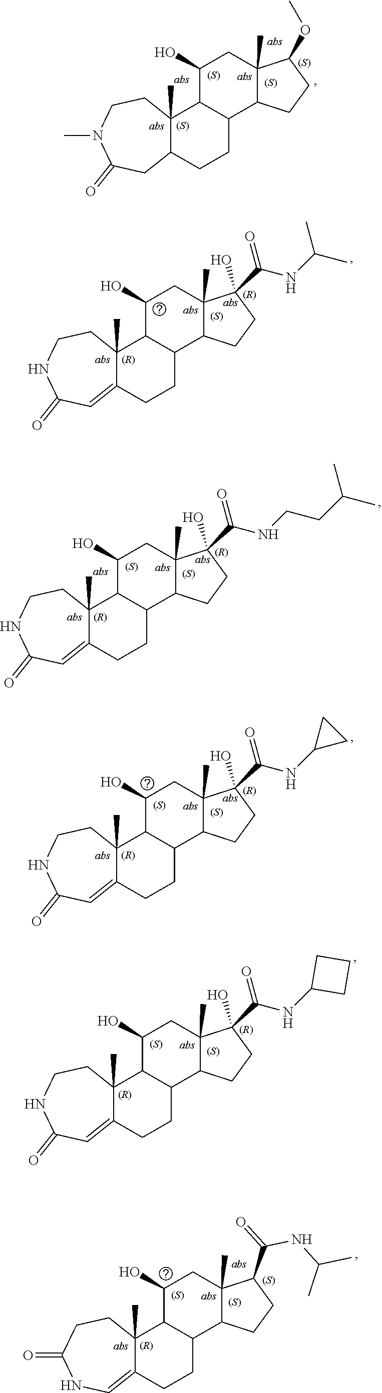

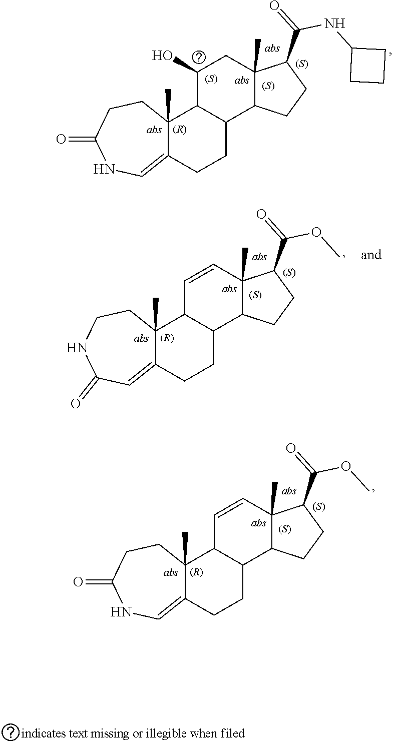

a pharmaceutically acceptable salt, stereoisomer or tautomer thereof.

In some embodiments, provided is a compound of formula (III):

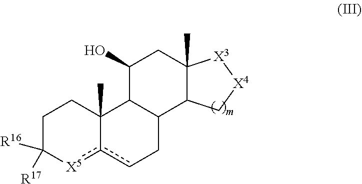

wherein
R¹⁶ and R¹⁷ together form oxo, or
R¹⁶ and R¹⁷ together with the carbon atom to which they are attached form a 5-membered heterocycle;
one of X³ and X⁴ is CH₂ or C═O, and the other is NR¹⁸;
X⁵ is CH₂, CH, or NR¹⁹;
R¹⁸ is H, C₁₋₆ alkyl, or C(O)R¹⁹.
each R¹⁹ is independently H or C₁₋₆ alkyl;
m is 1 or 2; and
the dashed lines represent optional double bonds, wherein the number of dashed lines representing double bonds is zero or one.

In some embodiments, R¹⁶ and R¹⁷ together form oxo. In some embodiments, R¹⁶ and R¹⁷ together with the carbon atom to which they are attached form a 5-membered heterocycle. In some embodiments, R¹⁶ and R¹⁷ together with the carbon atom to which they are attached form a dioxolane heterocycle. In some embodiments, X³ is CH₂, and X⁴ is NR¹⁸. In some embodiments, X³ is C═O, and X⁴ is NR¹⁸. In some embodiments, X⁴ is CH₂, and X³ is NR¹⁸. In some embodiments, X⁴ is C═O, and X³ is NR¹⁸.

In some embodiments, X⁵ is CH₂ or CH. In some embodiments, X⁵ is NR¹⁹. In some embodiments, R¹⁸ is H. In some embodiments, R¹⁸ is C₁₋₆ alkyl. In some embodiments, R¹⁸ is methyl. In some embodiments, R¹⁸ is C(O)R¹⁹. In some embodiments, R¹⁹ is H. In some embodiments, R¹⁹ is C₁₋₆ alkyl. In some embodiments, R¹⁹ is methyl.

In some embodiments, provided is a compound of formula (IIIa),

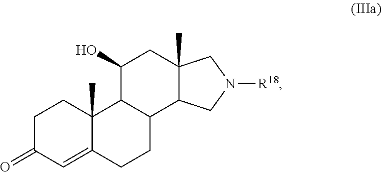

or a pharmaceutically acceptable salt, stereoisomer or tautomer thereof, wherein each variable is as defined for formula (II) or any variation thereof.

In some embodiments, R¹⁸ is methyl. In some embodiments, R¹⁸ is acetyl.

In some embodiments, provided is a compound of formula (IIIb),

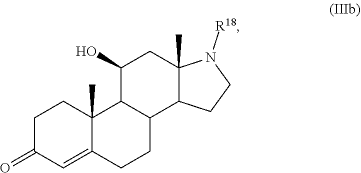

or a pharmaceutically acceptable salt, stereoisomer or tautomer thereof, wherein each variable is as defined for formula (II) or any variation thereof.

In some embodiments, R⁸ is methyl. In some embodiments, R¹⁸ is acetyl.

In some embodiments, provided is a compound of formula (IIIc),

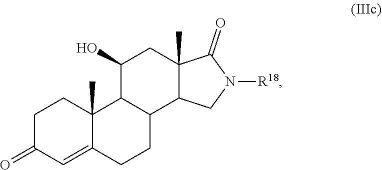

or a pharmaceutically acceptable salt, stereoisomer or tautomer thereof, wherein each variable is as defined for formula (II) or any variation thereof.

In some embodiments, R⁸ is H. In some embodiments, R⁸ is methyl.

In some embodiments, provided is a compound of formula (IIId),

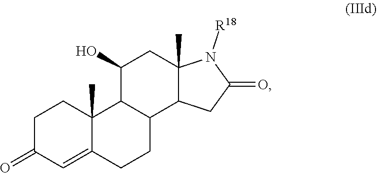

or a pharmaceutically acceptable salt, stereoisomer or tautomer thereof, wherein each variable is as defined for formula (II) or any variation thereof.

In some embodiments, R¹⁸ is acetyl. In some embodiments, R¹⁸ is methyl.

In some embodiments, provided is a compound of formula (IIIe),

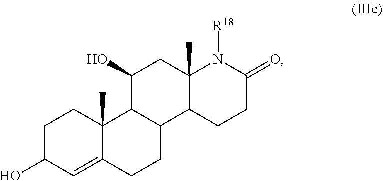

or a pharmaceutically acceptable salt, stereoisomer or tautomer thereof, wherein each variable is as defined for formula (II) or any variation thereof.

In some embodiments, R¹⁸ is H. In some embodiments, R¹⁸ is methyl.

In some embodiments, provided is a compound selected from the group consisting of

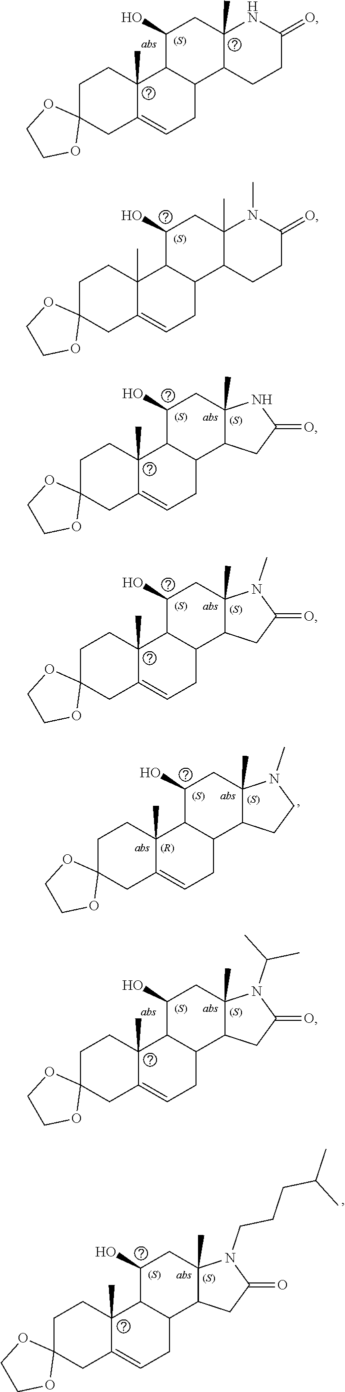

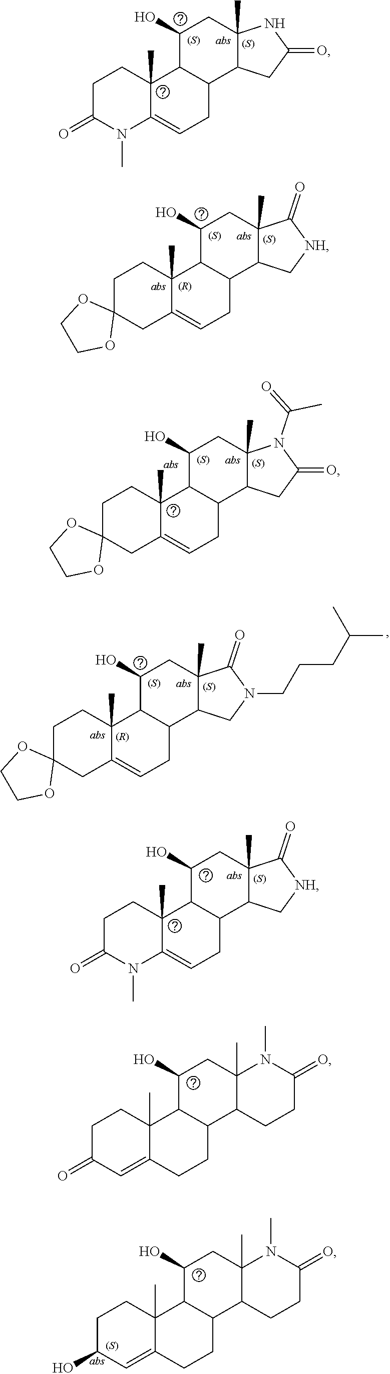

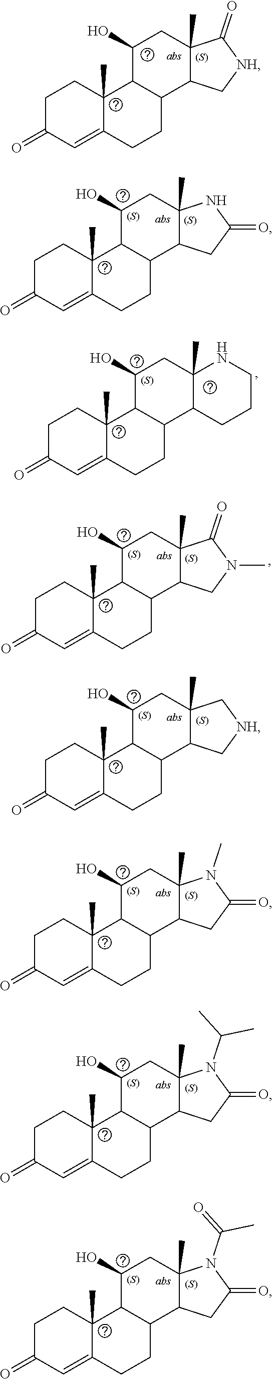

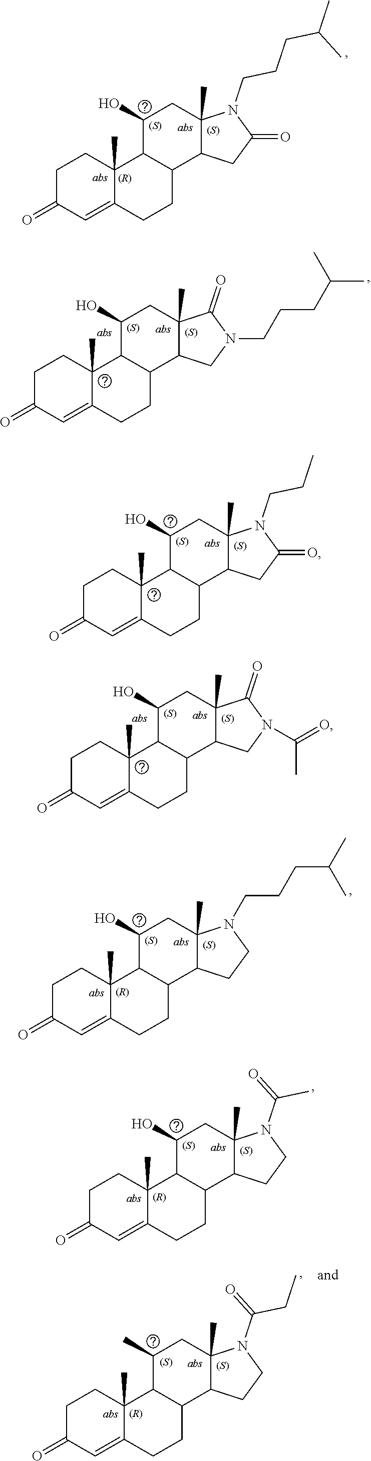

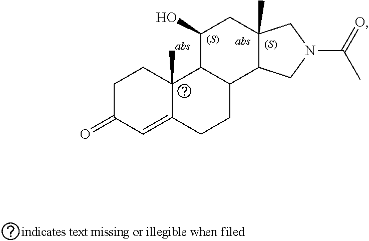

or a pharmaceutically acceptable salt, isomer, or tautomer thereof.

In some aspects, provided is a compound selected from the group consisting of

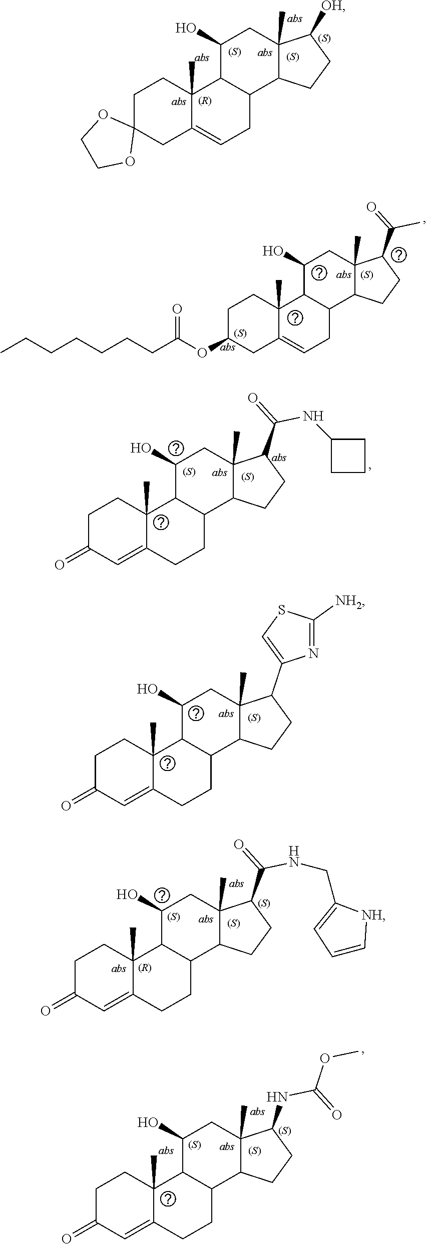

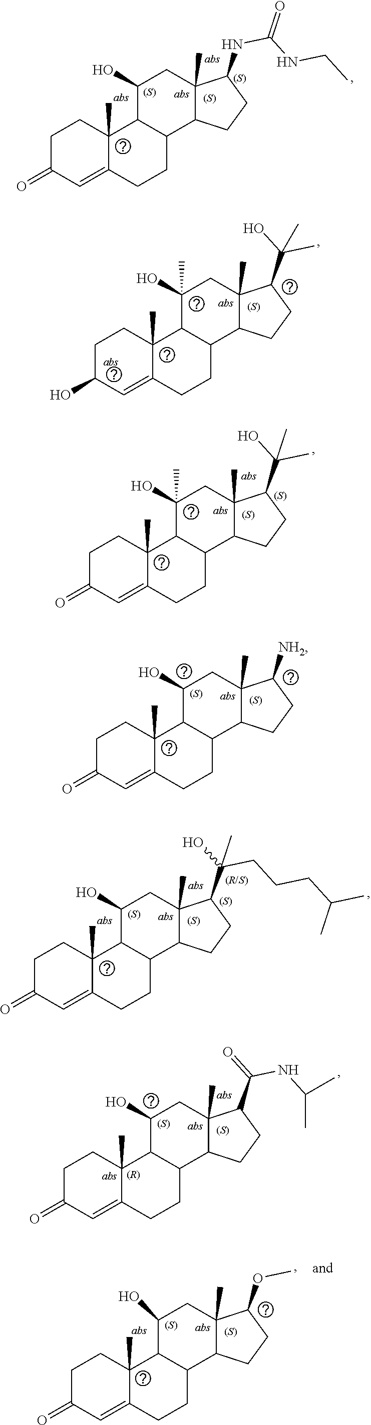

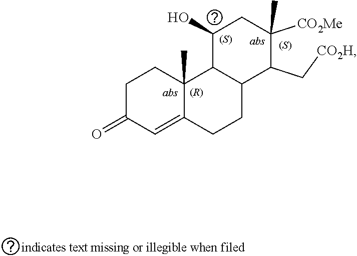

or a pharmaceutically acceptable salt, isomer, or tautomer thereof.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of R², R³, and/or R⁴ the same as if each and every combination were specifically and individually listed. Every description, variation, embodiment or aspect provided herein with respect to X¹ of formula (I) may be combined with every description, variation, embodiment or aspect of X², R¹¹, R¹², and/or R¹⁵ the same as if each and every combination were specifically and individually listed.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity. In some embodiments, the provided compounds are sterilized.

Representative compounds are listed in Table 1.

| Comp. | | |
|---|---|---|
| Number | Structure | Chemical Name |
| 1 | 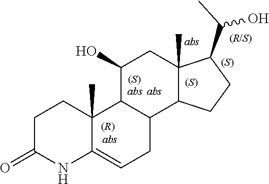 | (4aR,5S,6aS,7S)-5-hydroxy-7-((S)- 1-hydroxyethyl)-4a,6a-dimethyl- 1,3,4,4a,4b,5,6,6a,7,8,9,9a,9b,10- tetradecahydro-2H-indeno[5,4- f]quinolin-2-one |
| 2 | 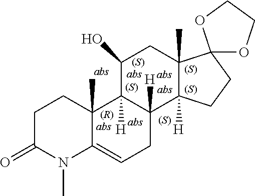 | |
| 3 | 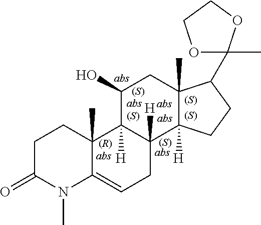 | (4aR,5S,6aS)-5-hydroxy-1,4a,6a- trimethyl-7-(2-methyl-1,3- dioxolan-2-yl)- 1,3,4,4a,4b,5,6,6a,7,8,9,9a,9b,10- tetradecahydro-2H-indeno[5,4- f]quinolin-2-one |
| 4 | 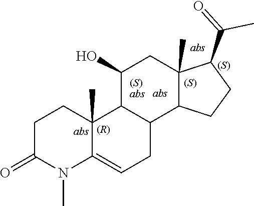 | (4aR,5S,6aS,7S)-7-acetyl-5- hydroxy-1,4a,6a-trimethyl- 1,3,4,4a,4b,5,6,6a,7,8,9,9a,9b,10- tetradecahydro-2H-indeno[5,4- f]quinolin-2-one |
| 5 | 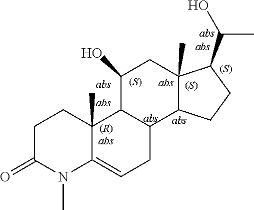 | |
| 6 | 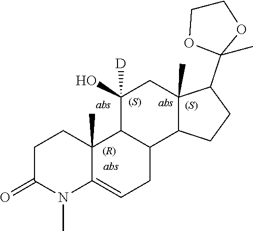 | |
| 7 | 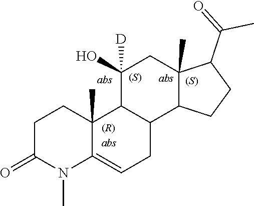 | |
| 8 | 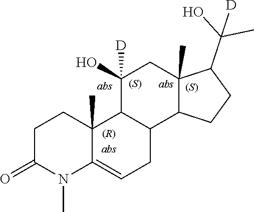 | |
| 9 | 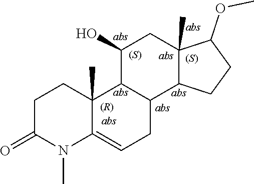 | |
| 10 | 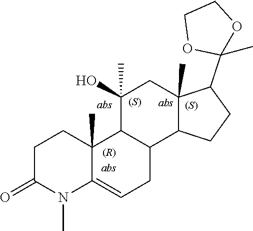 | |
| 11 | 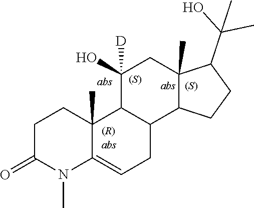 | |
| 12 | 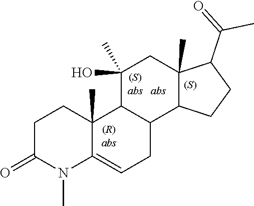 | (4aR,5S,6aS)-7-acetyl-5-hydroxy- 1,4a,5,6a-tetramethyl- 1,3,4,4a,4b,5,6,6a,7,8,9,9a,9b,10- tetradecahydro-2H-indeno[5,4- f]quinolin-2-one |
| 13 | 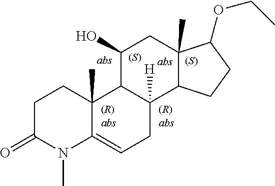 | |
| 14 | 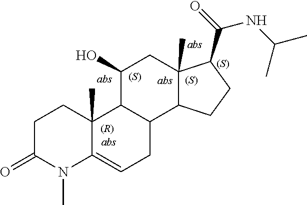 | |
| 15 | 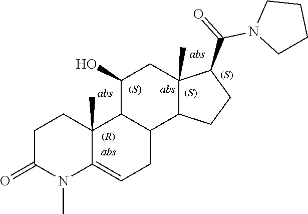 | |
| 16 | 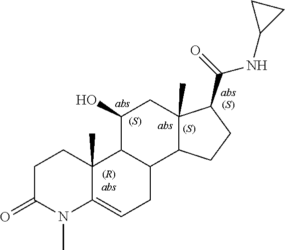 | |
| 17 | 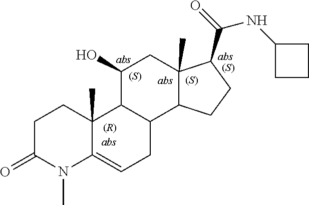 | (4aR,5S,6aS,7S)-N-cyclobutyl-5- hydroxy-1,4a,6a-trimethyl-2-oxo- 2,3,4,4a,4b,5,6,6a,7,8,9,9a,9b,10- tetradecahydro-1H-indeno[5,4- f]quinoline-7-carboxamide |
| 18 | 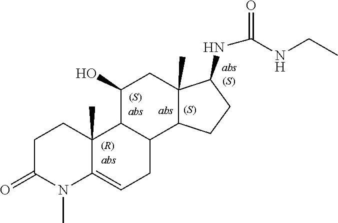 | |
| 19 | 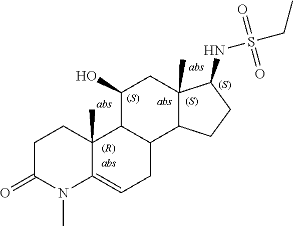 | N-((4aR,5S,6aS,7S)-5-hydroxy- 1,4a,6a-trimethyl-2-oxo- 2,3,4,4a,4b,5,6,6a,7,8,9,9a,9b,10- tetradecahydro-1H-indeno[5,4- f]quinolin-7-yl)ethanesulfonamide |
| 20 | 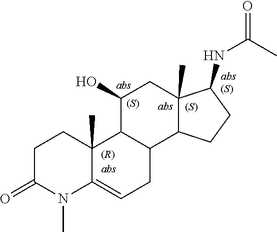 | |
| 21 | 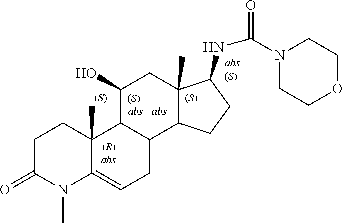 | |
| 22 | 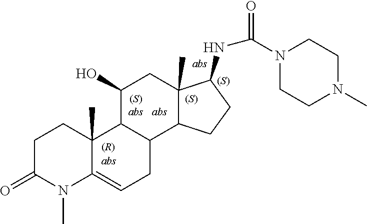 | |
| 23 | 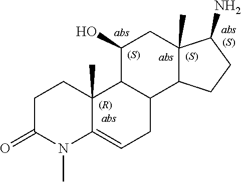 | (4aR,5S,6aS,7S)-7-amino-5- hydroxy-1,4a,6a-trimethyl- 1,3,4,4a,4b,5,6,6a,7,8,9,9a,9b,10- tetradecahydro-2H-indeno[5,4- f]quinolin-2-one |
| 24 | 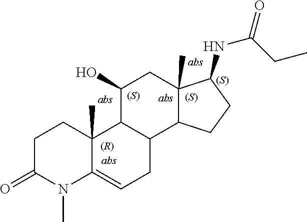 | |
| 25 | 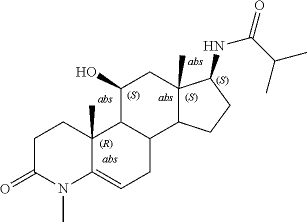 | |
| 26 | 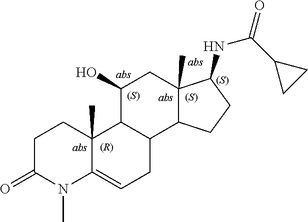 | N-((4aR,5S,6aS,7S)-5-hydroxy- 1,4a,6a-trimethyl-2-oxo- 2,3,4,4a,4b,5,6,6a,7,8,9,9a,9b,10- tetradecahydro-1H-indeno[5,4- f]quinolin-7- yl)cyclopropanecarboxamide |
| 27 | 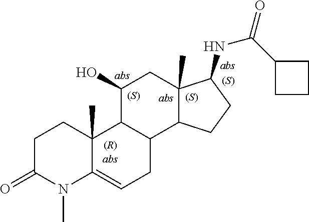 | N-((4aR,5S,6aS,7S)-5-hydroxy- 1,4a,6a-trimethyl-2-oxo- 2,3,4,4a,4b,5,6,6a,7,8,9,9a,9b,10- tetradecahydro-1H-indeno[5,4- f]quinolin-7- yl)cyclobutanecarboxamide |
| 28 | 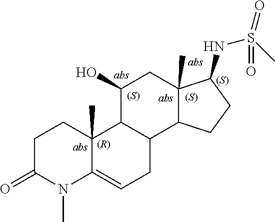 | |
| 29 | 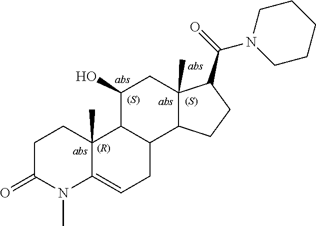 | |
| 30 | 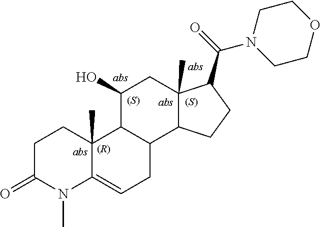 | |
| 31 | 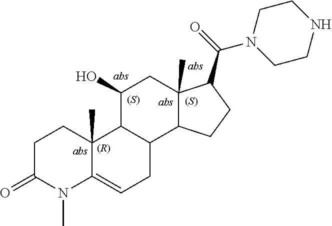 | |
| 32 | 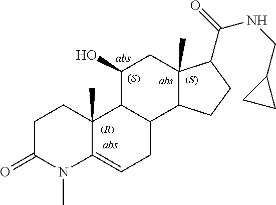 | (4aR,5S,6aS)-N- (cyclopropylmethyl)-5-hydroxy- 1,4a,6a-trimethyl-2-oxo- 2,3,4,4a,4b,5,6,6a,7,8,9,9a,9b,10- tetradecahydro-1H-indeno[5,4- f]quinoline-7-carboxamide |
| 33 | 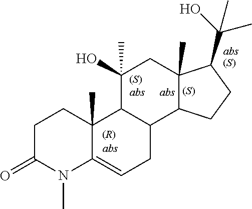 | |
| 34 | 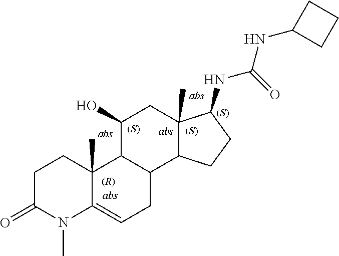 | |
| 35 | 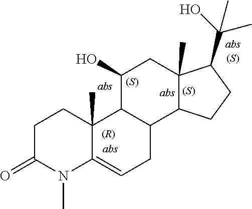 | |
| 36 | 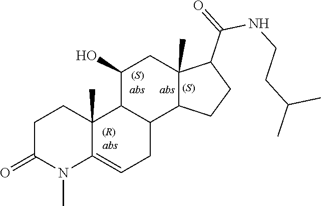 | (4aR,5S,6aS)-5-hydroxy-N- isopentyl-1,4a,6a-trimethyl-2-oxo- 2,3,4,4a,4b,5,6,6a,7,8,9,9a,9b,10- tetradecahydro-1H-indeno[5,4- f]quinoline-7-carboxamide |
| 37 | 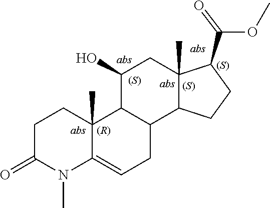 | |
| 38 | 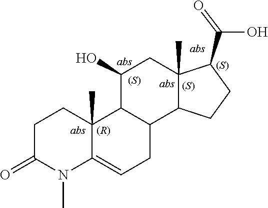 | (4aR,5S,6aS,7S)-5-hydroxy- 1,4a,6a-trimethyl-2-oxo- 2,3,4,4a,4b,5,6,6a,7,8,9,9a,9b,10- tetradecahydro-1H-indeno[5,4- f]quinoline-7-carboxylic acid |
| 39 | 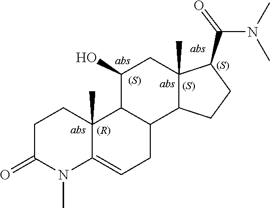 | |
| 40 | 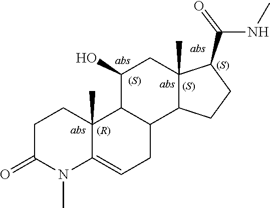 | (4aR,5S,6aS)-5-hydroxy-N,1,4a,6a- tetramethyl-2-oxo- 2,3,4,4a,4b,5,6,6a,7,8,9,9a,9b,10- tetradecahydro-1H-indeno[5,4-f] quinoline-7-carboxamide |
| 41 | 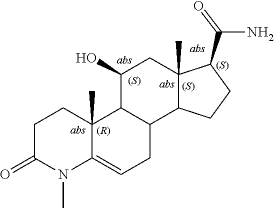 | |
| 42 | 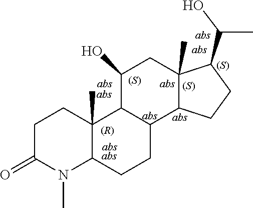 | |
| 43 | 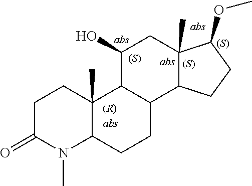 | (4aR,5S,6aS,7S)-5-hydroxy-7- methoxy-1,4a,6a- trimethylhexadecahydro-2H- indeno[5,4-f]quinolin-2-one |
| 44 | 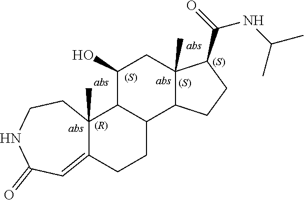 | |
| 45 | 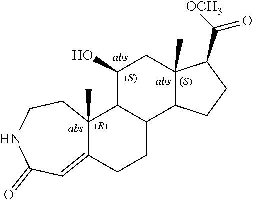 | methyl(5aR,6S,7aS)-6-hydroxy- 5a,7a-dimethyl-2-oxo- 2,3,4,5,5a,5b,6,7,7a,8,9,10,10a,10b, 11,12- hexadecahydrocyclopenta[5,6] naphtho[1,2-d]azepine-8-carboxylate |
| 46 | 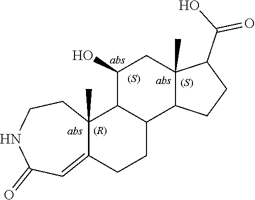 | (5aR,6S,7aS)-6-hydroxy-5a,7a- dimethyl-2-oxo- 2,3,4,5,5a,5b,6,7,7a,8,9,10,10a,10b, 11,12- hexadecahydrocyclopenta[5,6]naphtho [1,2-d]azepine-8-carboxylic acid |
| 47 | 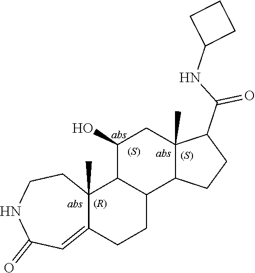 | (5aR,6S,7aS)-N-cyclobutyl-6- hydroxy-5a,7a-dimethyl-2-oxo- 2,3,4,5,5a,5b,6,7,7a,8,9,10,10a,10b, 11,12- hexadecahydrocyclopenta[5,6] naphtho[1,2-d]azepine-8-carboxamide |
| 48 | 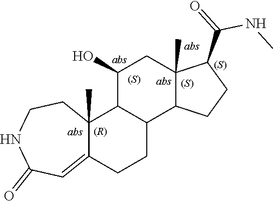 | |
| 49 | 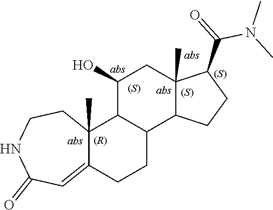 | (5aR,6S,7aS,8S)-6-hydroxy- N,N,5a,7a-tetramethyl-2-oxo- 2,3,4,5,5a,5b,6,7,7a,8,9,10,10a,10b, 11,12- hexadecahydrocyclopenta[5,6] naphtho[1,2-d]azepine-8-carboxamide |
| 50 | 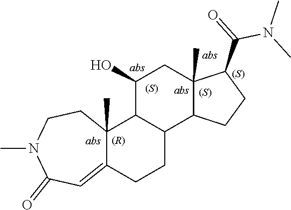 | N-((4aR,5S,6aS,7S)-5-hydroxy- 1,4a,6a-trimethyl-2-oxo- 2,3,4,4a,4b,5,6,6a,7,8,9,9a,9b,10- tetradecahydro-1H-indeno[5,4- f]quinolin-7- yl)cyclopropanecarboxamide |
| 51 | 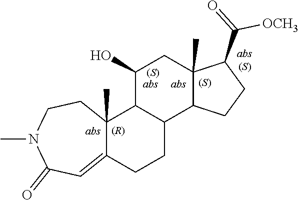 | methyl(5aR,6S,7aS,8S)-6- hydroxy-3,5a,7a-trimethyl-2-oxo- 2,3,4,5,5a,5b,6,7,7a,8,9,10,10a,10b, 11,12- hexadecahydrocyclopenta[5,6] naphtho[1,2-d]azepine-8-carboxylate |
| 52 | 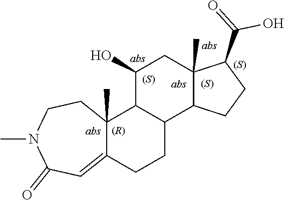 | |
| 53 | 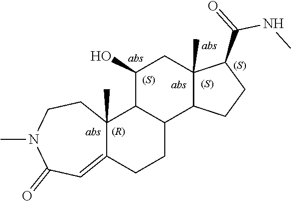 | |
| 54 | 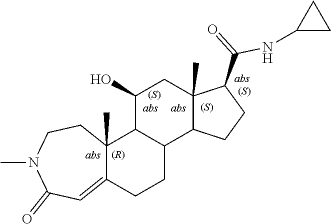 | (5aR,6S,7aS,8S)-N-cyclopropyl-6- hydroxy-3,5a,7a-trimethyl-2-oxo- 2,3,4,5,5a,5b,6,7,7a,8,9,10,10a,10b, 11,12- hexadecahydrocyclopenta[5,6] naphtho[1,2-d]azepine-8-carboxamide |
| 55 | 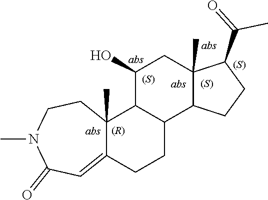 | |
| 56 | 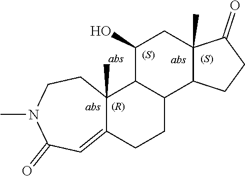 | |
| 57 | 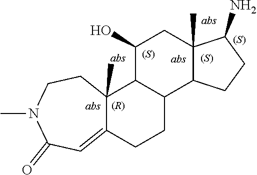 | |
| 58 | 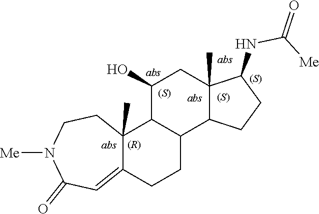 | |
| 59 | 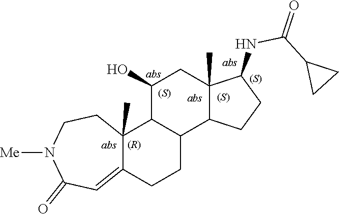 | N-((5aR,6S,7aS,8S)-6-hydroxy- 3,5a,7a-trimethyl-2-oxo- 2,3,4,5,5a,5b,6,7,7a,8,9,10,10a,10b, 11,12- hexadecahydrocyclopenta[5,6] naphtho[1,2-d]azepin-8- yl)cyclopropanecarboxamide |
| 60 | 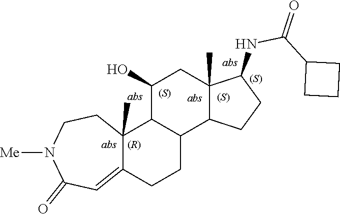 | N-((5aR,6S,7aS,8S)-6-hydroxy- 3,5a,7a-trimethyl-2-oxo- 2,3,4,5,5a,5b,6,7,7a,8,9,10,10a,10b, 11,12- hexadecahydrocyclopenta[5,6] naphtho[1,2-d]azepin-8- yl)cyclobutanecarboxamide |
| 61 | 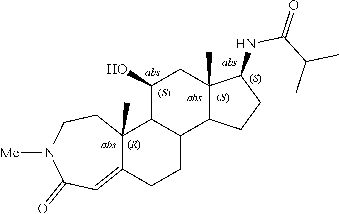 | |
| 62 | 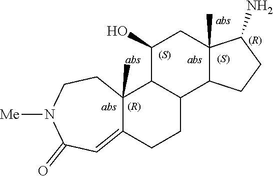 | |
| 63 | 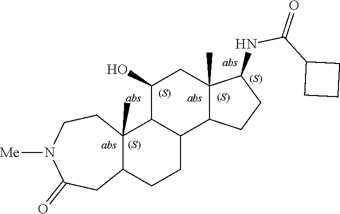 | |
| 64 | 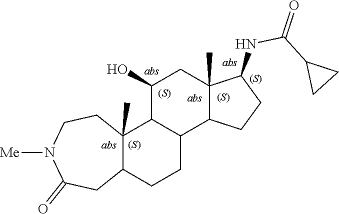 | |
| 65 | 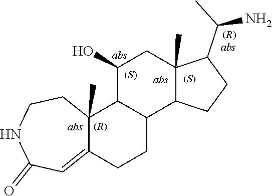 | |
| 66 | 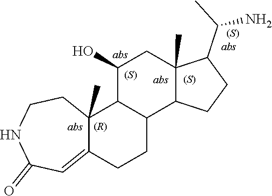 | |
| 67 | 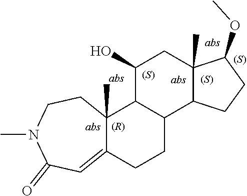 | |
| 68 | 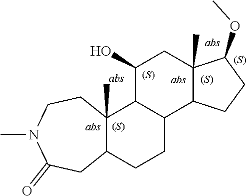 | |
| 69 | 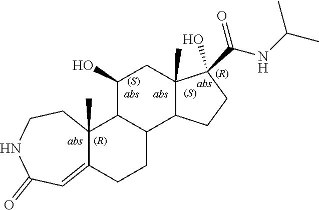 | |
| 70 | 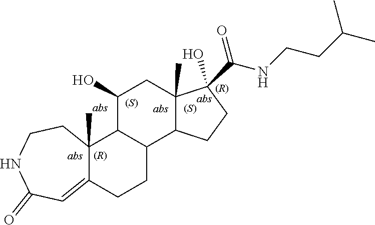 | |
| 71 | 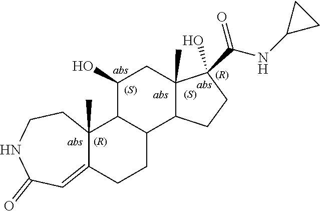 | |
| 72 | 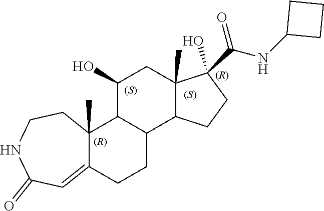 | |
| 73 | 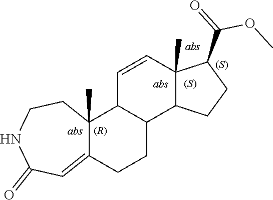 | methyl(5aR,7aS,8S)-5a,7a- dimethyl-2-oxo- 2,3,4,5,5a,5b,7a,8,9,10,10a,10b,11, 12- tetradecahydrocyclopenta[5,6] naphtho[1,2-d]azepine-8-carboxylate |
| 74 | 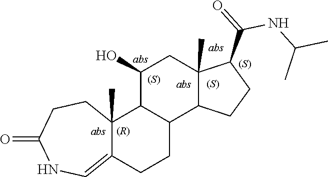 | (5aR,6S,7aS,8S)-6-hydroxy-N- isopropyl-5a,7a-dimethyl-3-oxo- 2,3,4,5,5a,5b,6,7,7a,8,9,10,10a,10b, 11,12- hexadecahydrocyclopenta[5,6] naphtho[2,1-c]azepine-8-carboxamide |
| 75 | 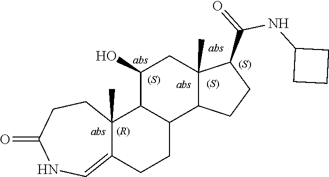 | |
| 76 | 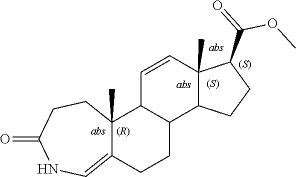 | |
| 77 | 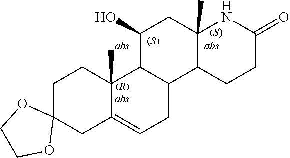 | |
| 78 | 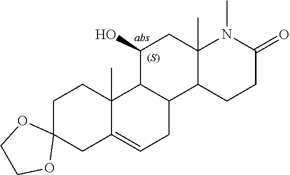 | |
| 79 | 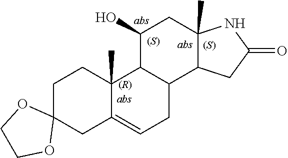 | |
| 80 | 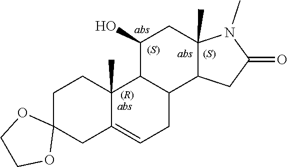 | |
| 81 | 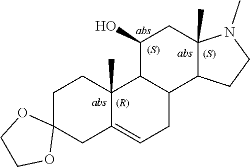 | |
| 82 | 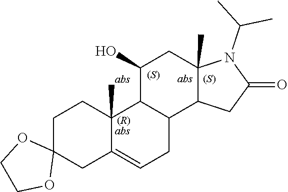 | |
| 83 | 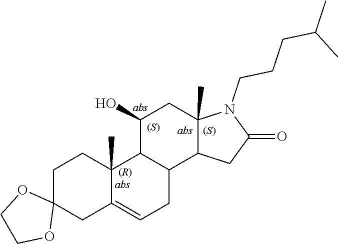 | |
| 84 | 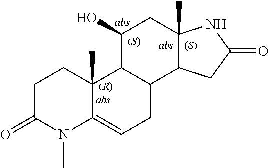 | |
| 85 | 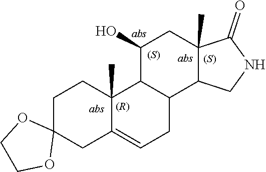 | |
| 86 | 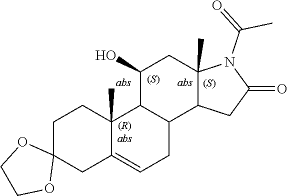 | |
| 87 | 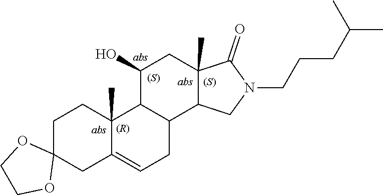 | |
| 88 | 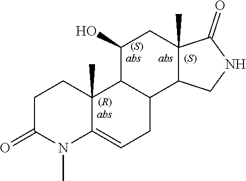 | |
| 89 | 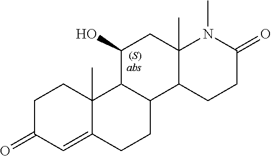 | |
| 90 | 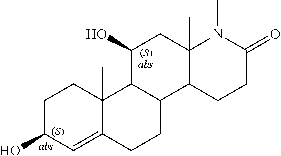 | |
| 91 | 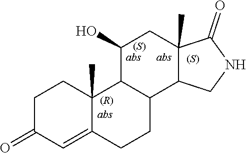 | |
| 92 | 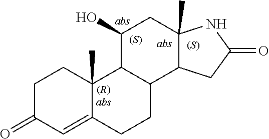 | |
| 93 | 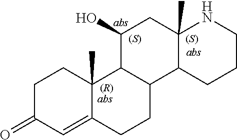 | |
| 94 | 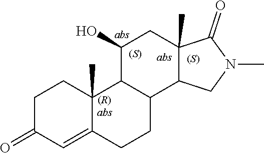 | |
| 95 | 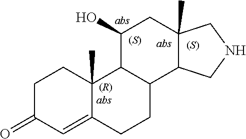 | |
| 96 | 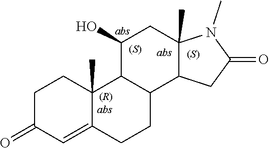 | |
| 97 | 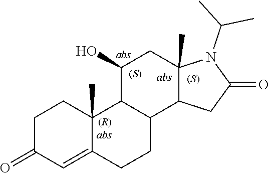 | |
| 98 | 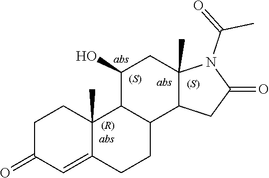 | |
| 99 | 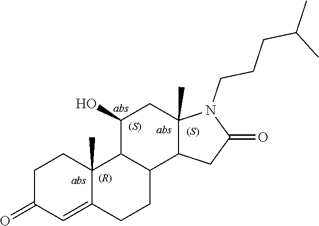 | |
| 100 | 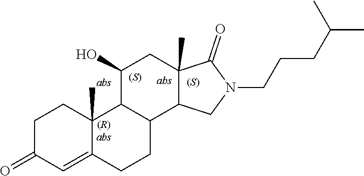 | |
| 101 | 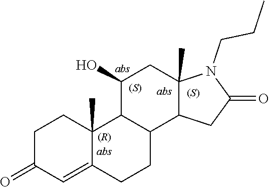 | |
| 102 | 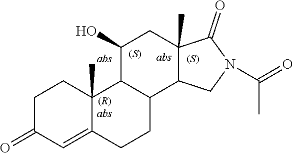 | |
| 103 | 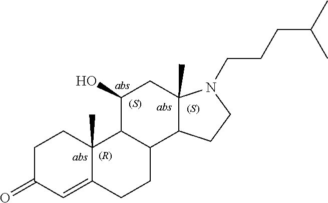 | |
| 104 | 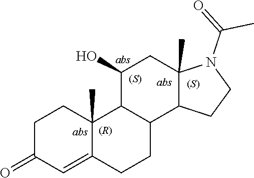 | |
| 105 | 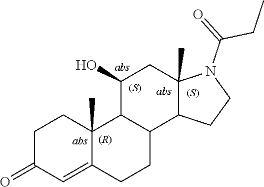 | |
| 106 | 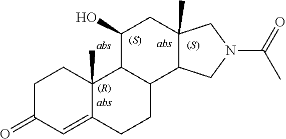 | |
| 107 | 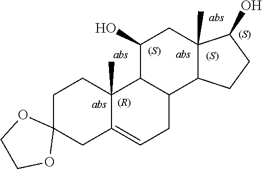 | |
| 108 | 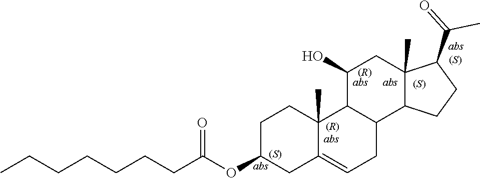 | |
| 109 | 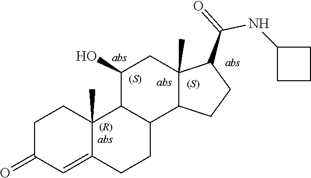 | |
| 110 | 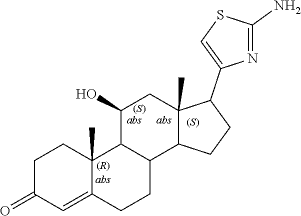 | |
| 111 | 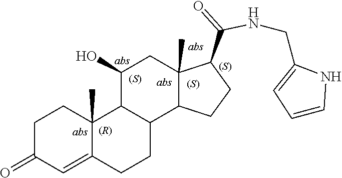 | |
| 112 | 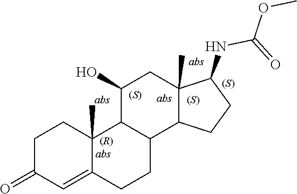 | |
| 113 | 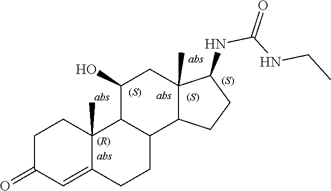 | |
| 114 | 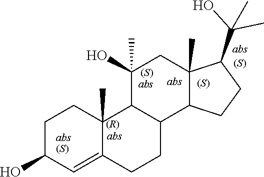 | |
| 115 | 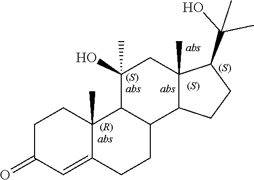 | |
| 116 | 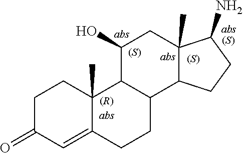 | |
| 117 | 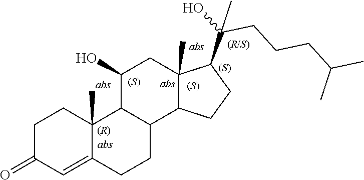 | |
| 118 | 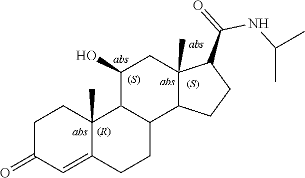 | |
| 119 | 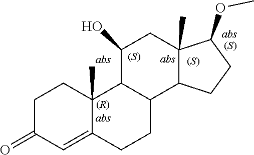 | |
| 120 | 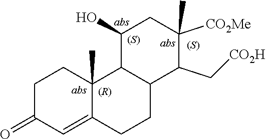 | |

Provided herein is a compound selected from the group consisting of the compounds described in Table 1.

Also provided herein are, where applicable, are any and all stereoisomers of the compounds depicted herein, including compounds of formulae I and II, including geometric isomers (e.g., cis/trans isomers or E/Z isomers), enantiomers, diastereomers, or mixtures thereof in any ratio, including racemic mixtures. In some embodiments, a provided compound has two stereocenters which are in the cis configuration. In some embodiments, a provided compound has two stereocenters which are in the trans configuration. In some embodiments, a provided compound has two stereocenters in the (S, S) configuration. In some embodiments, a provided compound has two stereocenters in the (R, R) configuration. In some embodiments, a provided compound has two stereocenters in the (S, R) configuration. In some embodiments, a provided compound has two stereocenters in the (R, S) configuration. In some embodiments, a provided compound is present at 75% stereoisomeric purity in a mixture of enantiomers and/or diastereomers. In some embodiments, a provided compound is present at 80% stereoisomeric purity in a mixture of enantiomers and/or diastereomers. In some embodiments, a provided compound is present at 90% stereoisomeric purity in a mixture of enantiomers and/or diastereomers. In some embodiments, a provided compound is present at 95% stereoisomeric purity in a mixture of enantiomers and/or diastereomers. In some embodiments, a provided compound is present at 99% stereoisomeric purity in a mixture of enantiomers and/or diastereomers. In some embodiments, a provided compound is present at 99.5% stereoisomeric purity in a mixture of enantiomers and/or diastereomers. In some embodiments, a provided compound is present at 99.9% stereoisomeric purity in a mixture of enantiomers and/or diastereomers. In some embodiments, a provided compound is a single stereoisomer that is substantially free of other enantiomers and/or diastereomers.

It is understood that compounds with tautomeric forms are described and embraced herein. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The compounds described here also intend isotopically-labeled and/or isotopically-enriched forms. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into the provided compounds include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²p, ³⁵S, ¹⁸F, ³⁶Cl. Certain isotope labeled compounds (e.g. ³H and ¹⁴C) is useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds can generally be prepared by standard methods and techniques known to those skilled in the art or by procedures similar to those described in the accompanying Examples substituting appropriate isotopically-labeled reagents in place of the corresponding non-labeled reagent.

Compositions of any of the compounds detailed herein are embraced by this disclosure. In some embodiments, the provided are pharmaceutical compositions comprising a compound as detailed herein or a salt thereof and a pharmaceutically acceptable carrier or excipient.

### Methods of Preparation

The provided compounds may be prepared by a number of processes, including but not limited to the processes generally described below. In the following process descriptions, the symbols when used in the formulae depicted are to be understood to represent those groups described above in relation to the formulae herein.

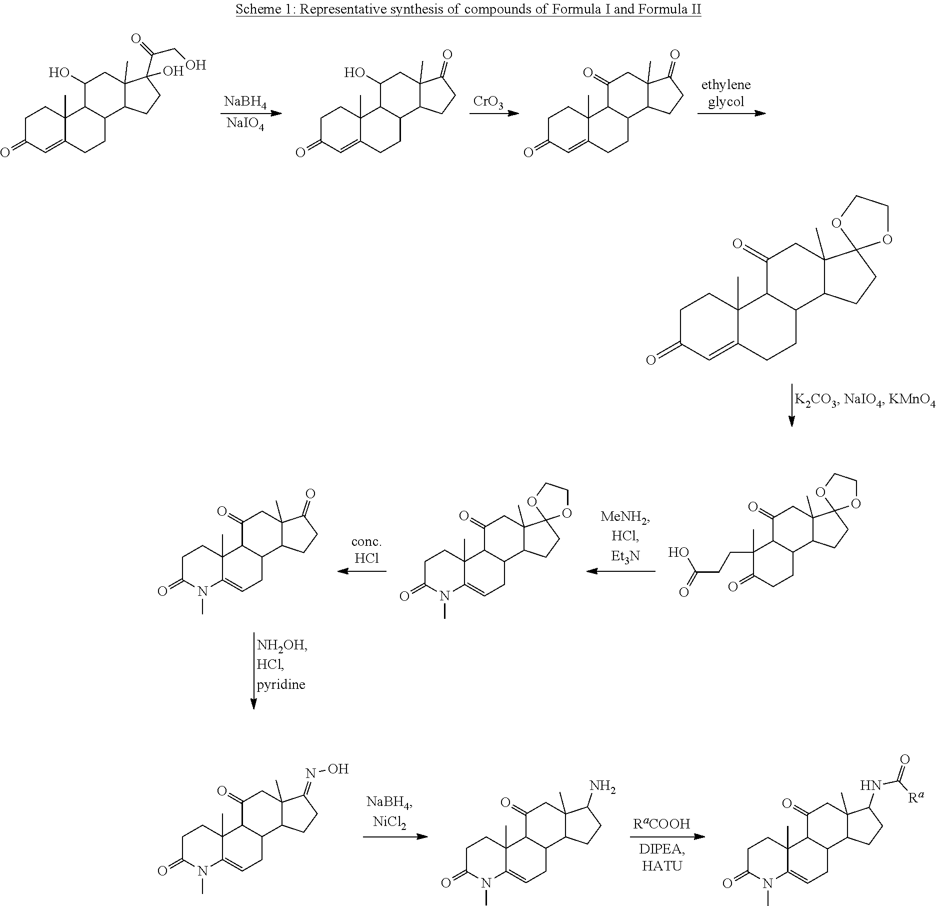

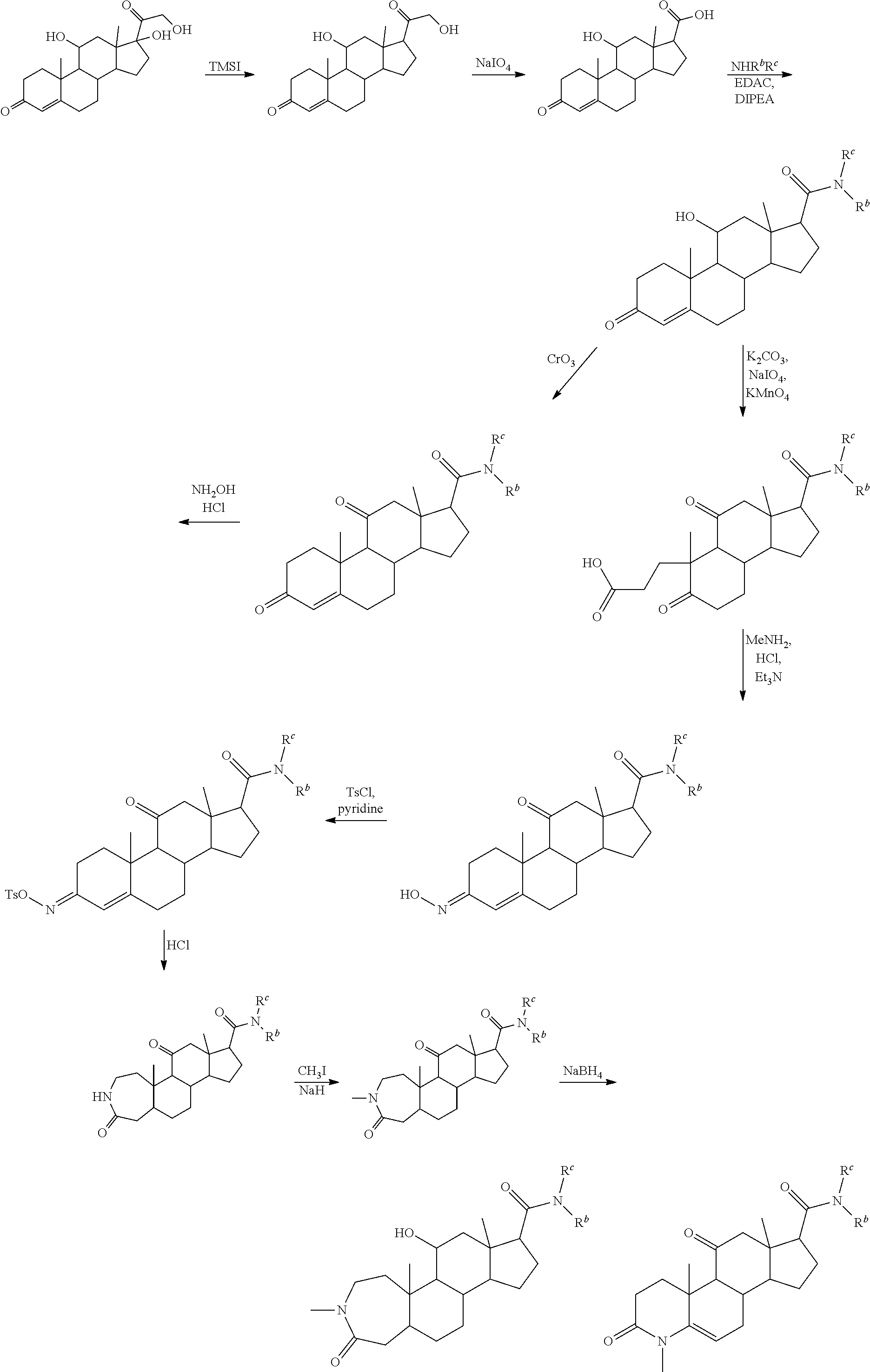

### Methods of Use

In certain embodiments, disclosed herein is a method of treatment of a mitochondrial biogenesis-mediated disease comprising the administration of a therapeutically effective amount of a compound as disclosed herein to a patient in need thereof.

In some embodiments, the mitochondrial biogenesis-mediated disease is selected from the group consisting of skeletal or cardiac muscle diseases associated with ischemia, or impaired or inadequate blood flow, diseases associated with genetic disorders that directly or indirectly affect the number, structure, or function of mitochondria, diseases associated with impaired neurological function associated with decreased mitochondrial number or function, diseases associated with loss of number, loss of function, or loss of correct, optimally efficient internal organization of skeletal muscle cells or cardiac muscle cells, metabolic diseases, and conditions associated with liver cell injury and altered fatty acid metabolism.

In some embodiments, the mitochondrial biogenesis-mediated disease is selected from the group consisting of acute coronary syndrome, myocardial infarction, angina, renal injury, renal ischemia, diseases of the aorta and its branches, injuries arising from medical interventions, atherosclerosis, trauma, diabetes, hyperlipidemia, vascular stenosis, peripheral arterial disease, vasculopathy, and vasculitis.

In some embodiments, the mitochondrial biogenesis-mediated disease is selected from the group consisting of Friedreich's ataxia, muscular dystrophy, Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, spinal muscular atrophy, and Emery-Dreifuss muscular dystrophy.

In some embodiments, the mitochondrial biogenesis-mediated disease is selected from the group consisting of Huntington's disease, Parkinson's disease, Alzheimer's disease, and amyotrophic lateral sclerosis.

In some embodiments, the mitochondrial biogenesis-mediated disease is sarcopenia.

In some embodiments, the mitochondrial biogenesis-mediated disease is selected from the group consisting of congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment.

In some embodiments, the mitochondrial biogenesis-mediated disease is selected from the group consisting of hepatic steatosis, hepatic fibrosis, cirrhosis, and hepatocyte or stellate cell injury.

In further embodiments, said method further comprises the administration of another therapeutic agent.

In some embodiments, the said agent is selected from the group consisting of hormones which stimulate muscle cell growth, γ-amino butyric acid or its derivatives, dietary protein supplements, anabolic steroids, biological factors known to enhance the growth, strength, endurance, or metabolism of skeletal or cardiac muscle, or recovery of skeletal muscle or cardiac muscle from injury or weakness, compounds known to be associated with increased nitric oxide production which promotes blood flow through muscles, extracts of natural products known to promote muscle strength or endurance, inhibitors of myostatin, stimulators of follistatin expression, compounds known to promote or facilitate mitochochondrial function or biogenesis, a tetracycline antibiotic, glycoprotein IIb/IIIa inhibitor, ADP receptor/P2Y12 inhibitor, prostaglandin analog, COX inhibitor, antiplatelet drug, anticoagulant, heparin, direct factor Xa inhibitor, direct thrombin (II) inhibitor, vasodilator.

In some embodiments, the said agent is doxycycline.

In some embodiments, the said agent is niacin or allopurinol.

In certain embodiments, disclosed herein are methods of treating or preventing the adverse effects of administration of compounds which exhibit mitochondrial toxicity comprising the administration of a therapeutically effective amount of a compound as disclosed herein to a patient in need thereof.

In some embodiments, the adverse effect is selected from the group consisting of abnormal mitochondrial respiration, abnormal oxygen consumption, abnormal extracellular acidification rate, abnormal mitochondrial number, abnormal lactate accumulation, and abnormal ATP levels.

In certain embodiments, disclosed herein are methods of improving muscle structure or function; improving mitochondrial effects associated with exercise; enhancing the capacity for exercise in those limited by age, inactivity, diet, or diseases; enhancing muscle health and function in response to exercise; enhancing muscle health and function in the clinical setting of restricted capacity for exercise; enhancing recovery of muscles from vigorous activity or from injury associated with vigorous or sustained activity, comprising the administration of a therapeutically effective amount of a compound as disclosed herein to a patient in need thereof.

In certain embodiments, disclosed herein are methods of enhancing sports performance and endurance, building muscle shape and strength, or facilitating recovery from the muscle related side effects of training or competition comprising the administration of a therapeutically effective amount of a compound as disclosed herein to a patient in need thereof.

In certain embodiments, disclosed herein are methods of stimulating increased number or function of skeletal muscle cells or contractile muscle cells comprising the administration of a therapeutically effective amount of a compound as disclosed herein to a patient in need thereof.

In some embodiments, the said stimulation of muscle cells comprises stimulation of cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes.

In certain embodiments, disclosed herein is a method of modulation of mitochondrial biogenesis comprising contacting mitochondria with a compound as disclosed herein.

In some embodiments, the mitochondrial biogenesis-mediated diseases include sarcopenia, muscular dystrophy, neurodegenerative diseases, liver disease, acute or chronic kidney failure, congestive heart failure, chronic obstructive pulmonary disorder (COPD), peripheral vascular disease, pulmonary hypertension, hyperlipidemia, hypertension, and diabetes.

In some embodiments, provided is a method of administering a compound or composition disclosed herein in an amount effective to stimulate the function, recovery, or regeneration of mitochondria or mitochondrial proteins or function. In further embodiments, provided is methods for preventing or treating adverse events or diseases associated with impaired mitochondrial number or function.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

“Deuterated compounds” encompassed within the scope of the provided compounds are the compounds which have selective incorporation of deuterium in place of hydrogen.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that stereochemical isomeric forms, including diastereomeric, enantiomeric, and epimeric forms as well as d-isomers and l-isomers, and mixtures thereof are encompassed. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. All cis, trans, syn, anti, entgegen (E), and zusammen (Z) isomers as well as the appropriate mixtures thereof are included. Additionally, compounds may exist as tautomers; all tautomeric isomers are provided. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “muscular diseases” refers to diseases associated with impaired skeletal muscle or cardiac muscle cell number or function.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein. In certain embodiments, a combination of compounds is administered such that the clearance half-life of each compound from the body overlaps at least partially with one another. For example, a first pharmaceutical has a clearance half-life of 1 hour and is administered at time=0, and a second pharmaceutical has a clearance half-life of 1 hour and is administered at time=45 minutes.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “pregnenolone and other related steroids” as used herein refers to any compound which retains the ring structure and the 11-oxo moiety of 11-hydroxy-pregnenolone itself, but which contains one or more substituent groups relative to 11-oxo-pregenolone. The term also includes prodrugs which release 11-hydroxy-pregnenolone when administered to a subject. The term also includes active metabolites of 11β-hydroxypregnenolone such as 11β-hydroxyprogesterone.

The term “11β-hydroxypregnenolone” as used herein refers to a compound having the structural formula:

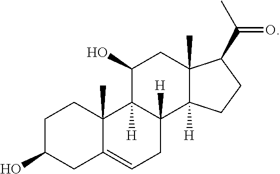

The term “11β-hydroxyprogesterone” as used herein refers to a compound having the structural formula:

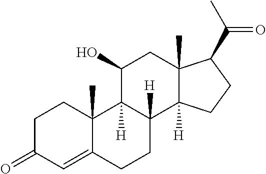

The term “derivative” as used herein to modify the term “11β-hydroxypregnenolone” or “11β-hydroxysreoid” the term “11β-hydroxyprogesterone” refers to any compound which retains the ring structure and stereochemistry of 11β-hydroxypregnenolone or 11β-hydroxyprogesterone, “11β-hydroxysreoid” itself, but which contains one or more substituent groups relative to 11β-hydroxypregnenolone or 11β-hydroxyprogesterone. The term also includes combination molecules or prodrugs that release 11β-hydroxypregnenolone when administered to a subject. Such a combination molecule may include, for example, 11β-hydroxypregnenolone and an agent joined by a hydrolysable linker group.

The term “HCAEC” as used herein refer to Human corniary Artery endothelial cells; the term “BCAEC” refers to bovine corniary Artery endothelial cells; and the term “GAPDH” refers to glyceraldehyde-3-phosphate dehydrogenase.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. Included are compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, sodium, potassium, magnesium, and calcium salts of the compounds disclosed herein, and the like, are contemplated.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the provided compounds to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Specific sustained release formulations of the compounds disclosed herein are described in U.S. Pat. No. 6,410,052, which is hereby incorporated by reference.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. In an embodiment, the provided compounds may be used in a dosage in the range of 0.01 to 100 mg/Kg.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the provided compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds with agents which allow or enhance improvements in the number, structure or function of skeletal muscle cells or cardiac muscle cells.

Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of compounds or compositions disclosed herein in combination with agents which allow or enhance improvements in the number, structure, or function of skeletal muscle cells or cardiac muscle cells, cofactors that enhance mitochondrial biogenesis, and factors that enhance the production of NO in response to the stimulation of eNOS or nNOS.

In further embodiments, such agents include hormones which stimulate muscle cell growth, γ-amino butyric acid or its derivatives, dietary protein supplements, anabolic steroids, biological factors known to enhance the growth, strength, endurance, or metabolism of skeletal or cardiac muscle, or recovery of skeletal muscle or cardiac muscle from injury or weakness, compounds known to be associated with increased nitric oxide production which promotes blood flow through muscles, extracts of natural products known to promote muscle strength or endurance, inhibitors of myostatin, and stimulators of folistatin expression.

In further embodiments, hormones which stimulate muscle cell growth include, but are not limited to, growth hormone, growth hormone analogs, growth hormone releasing peptides or analogs thereof, growth hormone secretagogues, growth hormone precursors, or other hormones such as somatatropin or mechano growth factor.

In further embodiments, γ-amino butyric acid derivatives include, but are not limited to, 4-amino-3-phenylbutyric acid and neurotransmitters that benefit muscles by modulating the pituitary gland.

In further embodiments, dietary protein supplements include, but are not limited to, casein, whey, soy, egg white, hemp, rice, and pea proteins, amino acids precursors or derivatives thereof with known attributes of potentiating muscle growth, such as leucine, valine, isovaline, beta alanine, glutamine, glutamine dipeptide, or glycocyamine.

In further embodiments anabolic steroids, include, but are not limited to, testosterone or related steroid compounds with muscle growth inducing properties, such as cyclostanazol or methadrostenol, prohomones or derivatives thereof, modulators of estrogen, and selective androgen receptor modulators (SARMS).

In further embodiments, biological factors known to enhance the growth, strength, endurance, or metabolism of skeletal or cardiac muscle, or recovery of skeletal muscle or cardiac muscle from injury or weakness, include, but are not limited to, alpha-lipoic acid, taurine, caffeine, magnesium, niacin, folic acid, ornithine, vitamin B6, B12, or D, aspartate, creatine and its diverse salts such creatine monohydrate, betaine, N-acetyl cysteine, beta-hydroxyl methyl butyrate, lecithin, choline, phospholipid mixtures, phosphatidyl serine, carnitine, L-carnitine, acetyl-L-camitine, and glycine proprionyl-L-carnitine.

In an embodiment, the compounds are used for the activation AMPK.

In an embodiment, the compounds are used for the subsequent activation transcription factors such as PCG-1 alpha associated with mitochondrial biogenisis.

In further embodiments, the compounds are used for the activation mitochondrial biogenisis and functions.

In further embodiments, the compounds are used for treatment of diseases associated with mitochondrial depletion and/or dysfunctionselected, but not limited to, the group consisting of skeletal muscle diseases, cardiac muscle diseases associated with ischemia, or impaired or inadequate blood flow, diseases associated with genetic disorders that directly or indirectly affect the number, structure, or function of mitochondria, diseases associated with impaired neurological function associated with decreased mitochondrial number or function, diseases associated with loss of number, loss of function, or loss of correct, optimally efficient internal organization of skeletal muscle cells or cardiac muscle cells, metabolic diseases, and conditions associated with liver cell injury and altered fatty acid metabolism.

In further embodiments, the compounds are used for mitochondrial biogenesis-mediated disease is selected from the group consisting of acute coronary syndrome, myocardial infarction, angina, renal injury, renal ischemia, diseases of the aorta and its branches, injuries arising from medical interventions, atherosclerosis, trauma, diabetes, hyperlipidemia, vascular stenosis, peripheral arterial disease, vasculopathy, and vasculitis, Friedreich's ataxia, muscular dystrophy, Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, spinal muscular atrophy, Emery-Dreifuss muscular dystrophy, Huntington's disease, Parkinson's disease, Alzheimer's disease, and amyotrophic lateral sclerosis, sarcopenia, congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment, hepatic steatosis, hepatic fibrosis, cirrhosis, and hepatocyte or stellate cell injury.

In further embodiments, compounds known to be associated with increased nitric oxide production which promotes blood flow through muscles include, but are not limited to, arginine and citrulline.

In further embodiments, compounds known to promote or facilitate mitochochondrial function or biogenesis, include, but are not limited to, alpha lipoic acid, resveratrol, coenzyme Q10 and its derivatives, forskalin, metformin, acetyl-carnitine, alpha tocopherol, pyruvate, choline, B vitamins, niacin, and biotin.

In further embodiments, extracts of natural products known to promote muscle strength or endurance, include, but are not limited to, guarana, geranium robertianum, cirsium ologophyllum, bauhinia purpureae, yohimbe, bacopa monniera, beet powder, rhodiola, or tea extracts.

In further embodiments, inhibitors of myostatin are proteins, antibodies, peptides, or small molecules.

In further embodiments, stimulators of follistatin expression or function are proteins, peptides, or small molecules.

In further embodiments, compounds disclosed herein may be administered in combination with another agent or agents such as niacin, or inhibitors of xanthine oxidase, such as allopurinol.

In further embodiments, compounds disclosed herein may be administered orally or parenterally.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating muscular diseases in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of muscular diseases.

The compositions may also be formulated as nutraceutical compositions. The term “nutraceutical composition” as used herein refers to a food product, foodstuff, dietary supplement, nutritional supplement or a supplement composition for a food product or a foodstuff comprising exogenously added compounds as disclosed herein. Details on techniques for formulation and administration of such compositions may be found in Remington, The Science and Practice of Pharmacy 21st Edition (Mack Publishing Co., Easton, PA) and Nielloud and Marti-Mestres, Pharmaceutical Emulsions and Suspensions: 2nd Edition (Marcel Dekker, Inc, New York).

As used herein, the term “food product” refers to any food or feed suitable for consumption by humans or animals. The food product may be a prepared and packaged food (e.g., mayonnaise, salad dressing, bread, grain bar, beverage, etc.) or an animal feed (e.g., extruded and pelleted animal feed, coarse mixed feed or pet food composition). As used herein, the term foodstuff refers to any substance fit for human or animal consumption.

Food products or foodstuffs are for example beverages such as nonalcoholic and alcoholic drinks as well as liquid preparation to be added to drinking water and liquid food, non-alcoholic drinks are for instance soft drinks, sport drinks, fruit juices, such as orange juice, apple juice, and grapefruit juice, lemonades, teas, near-water drinks, milk and other dairy drinks such as for example yogurt drinks, and diet drinks. In another embodiment food products or foodstuffs refer to solid or semi-solid foods comprising a provided compound. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

Animal feed including pet food compositions advantageously include food intended to supply necessary dietary requirements, as well as treats (e.g., dog biscuits) or other food supplements. The animal feed comprising the composition may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

The term “dietary supplement” refers to a small amount of a compound for supplementation of a human or animal diet packaged in single or multiple dose units.

Dietary supplements do not generally provide significant amounts of calories but may contain other micronutrients (e.g., vitamins or minerals). The term food products or foodstuffs also includes functional foods and prepared food products pre-packaged for human consumption.

The term nutritional supplement refers to a composition comprising a dietary supplement in combination with a source of calories. In some embodiments, nutritional supplements are meal replacements or supplements (e.g., nutrient or energy bars or nutrient beverages or concentrates).

Dietary supplements may be delivered in any suitable format. In certain embodiments, dietary supplements are formulated for oral delivery. The ingredients of the dietary supplement are contained in acceptable excipients and/or carriers for oral consumption. The actual form of the carrier, and thus, the dietary supplement itself, is not critical. The carrier may be a liquid, gel, gelcap, capsule, powder, solid tablet (coated or noncoated), tea, or the like.

Compounds and compositions disclosed herein are administered in an “effective amount.” This term is defined hereinafter. Unless dictated otherwise, explicitly or otherwise, an “effective amount” is not limited to a minimal amount sufficient to ameliorate a condition, or to an amount that results in an optimal or a maximal amelioration of the condition. In the case when two or more compounds are administered together, an effective amount of one such compound may not be, in and of itself, be an effective amount, but may be an effective amount when used together with additional compounds.

In certain embodiments, the effective amount is an amount which stimulates mitochondrial function in cells. Such stimulation of mitochondrial function in cells may comprise stimulation of one or more of mitochondrial respiration and mitochondrial biogenesis. The methods and compositions described herein can assist in prevention of the consequences of mitochondrial toxicity which has not yet occurred, as well as provide for the active therapy of mitochondrial toxicity that may have already occurred.

While the phrase “administered together” as used herein may refer to the provision of chemical compositions in the same pharmaceutical composition, the phrase as used herein is not intended to imply that this must be so. Rather, two or more chemical compositions are “administered together” if the T_{1/2} for the clearances of each composition from the body overlaps at least partially with one another. For example, if a first pharmaceutical has a T_{1/2} for clearance of 1 hour and is administered at time=0, and a second pharmaceutical has a T_{1/2} for clearance of 1 hour and is administered at time=45 minutes, such pharmaceuticals are considered administered together. Conversely, if the second drug is administered at time=2 hours, such pharmaceuticals are not considered administered together.

Routes of administration for the pharmaceutical compositions include parenteral and enteral routes. Enteral routes of administration include delivery by mouth (oral), nasal, rectal, and vaginal routes. Parenteral routes of administration include intravenous, intramuscular, subcutaneous, and intraperitoneal routes. When more than one pharmaceutical composition is being administered, each need not be administered by the same route. In particularly embodiments, 11β-hydroxypregnenolone, or a derivative or pharmaceutically acceptable salt thereof, is administered together intravenously with one or more tetracycline antibiotics such as doxycycline, most preferably in a single pharmaceutical composition.

In certain embodiments, the methods disclosed herein comprise the administration to cells at least 10 pM, at least 1.0 nM, or at least 100 nM of a compounds disclosed herein.

In further embodiments, the methods disclosed herein comprise the administration of compounds of the disclosure in a total daily dose of about 0.001 mg/kg/dose to about 10 mg/kg/dose, alternately from about 0.3 mg/kg/dose to about 3 mg/kg/dose. In another embodiment the dose range is from about 0.1 to about 1 mg/kg/day. Generally between about 0.01 mg and about 0.1 gram per day can be administered; alternately between about 2.5 mg and about 200 mg can be administered. The dose may be administered in as many divided doses as is convenient.

In further embodiments, the methods disclosed herein comprise the administration of compounds disclosed herein in a range of about 0.1 to about 100 mg per kg body weight.

In further embodiments, the desired concentration is maintained for at least 30 minutes, 1 hour, 3 hours, 6 hours, 12 hours, 24 hours, 48 hours, 72 hours, or more. In yet further embodiments, the desired concentration is achieved at least once during each 12-hour period over at least 24 hours, 48 hours, 72 hours, 1 week, one month, or more; or at least once during each 24-hour period over at least 48 hours, 72 hours, 1 week, one month, or more. In order to maintain a desired concentration for a desired time, multiple doses of one or more compounds may be employed. The dosing interval may be determined based on the clearance half-life for each compound of interest from the body.

In certain embodiments, disclosed herein are methods and compositions for the treatment of diseases associated with loss of number, function, or correct, optimally efficient internal organization of mitochondria within cells.

In further embodiments, disclosed herein are methods and compositions for the treatment of skeletal or cardiac muscle diseases associated with ischemia, or impaired or inadequate blood flow. Examples of such states include, but are not limited to, atherosclerosis, trauma, diabetes, vascular stenosis, peripheral arterial disease, vasculopathy, and vasculitis.

In further embodiments, disclosed herein are methods and compositions for the treatment of diseases associated with genetic disorders that directly or indirectly affect the number, structure, or function of mitochondria, particularly those associated with muscle dysfunction or myopathy. Examples of such states include, but are not limited to, the set of diseases broadly classified as muscular dystrophies and Friedreich's ataxia. In further embodiments, said muscular dystrophy is selected from the group consisting of Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

In further embodiments, disclosed herein are methods and compositions for the therapeutic treatment of diseases associated with impaired neurological function associated with decreased mitochondrial number or function. Examples include, but are not limited to, Huntington's disease, Parkinson's disease, Alzheimer's disease, and amyotrophic lateral sclerosis (ALS).

In certain embodiments, disclosed herein are methods and compositions for the treatment of diseases associated with loss of number, loss of function, or loss of correct, optimally efficient internal organization of skeletal muscle cells or cardiac muscle cells. Such diseases may eventuate in a state of functionally significant muscle wasting, which, in its most pronounced form, is termed sarcopenia. Sarcopenia may be secondary to a variety of disorders, including aging, muscular dystrophy, diabetes, or other abnormal metabolic conditions, infection, inflammation, autoimmune disease, cardiac dysfunction, or severe disuse syndromes, or inactivity associated with arthritis. Examples of such diseases include, but are not limited to, congestive heart failure, aging, myocarditis, myositis, polymyalgia rheumatic, polymyositis, HIV, cancer and/or the side effects of chemotherapy targeting the cancer, malnutrition, aging, inborn errors of metabolism, trauma, and stroke or other types of neurological impairment.

In certain embodiments, disclosed herein are methods and compositions for use to enhance sports performance and endurance, to build muscle shape and strength, and to facilitate recovery from the muscle related side effects of training or competition, such as soreness, weakness, cramping, pain, or injury.

In certain embodiments, a subject is selected for treatment with a compound or composition disclosed herein based on the occurrence of one or more physiological manifestations of skeletal or cardiac muscle injury or dysfunction in the subject. Such manifestations include elevations in biomarkers known to be related to injury of the heart or skeletal muscle. Examples of such biomarkers include, but are not limited to, elevated plasma levels of cardiac or skeletal muscle enzymes or proteins, such as myoglobin, troponin, or creatine phosphokinase, lactic acidosis, and elevated serum creatinine.

In certain embodiments, a compound or composition as disclosed herein is administered in an amount which stimulates increased number or function of skeletal muscle cells or contractile muscle cells. Such stimulation of muscle cells may comprise stimulation of one or more aspects of muscle cell function, including cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes. The methods and compositions described herein can assist in prevention of the consequences of muscle injury or dysfunction which have not yet occurred, as well as provide for the active therapy of muscle injury, dysfunction, or diseases which have already occurred.

In yet another aspect, provided is methods of treating metabolic disease in a subject. These methods comprise administering to a subject in need thereof a compound disclosed herein. In certain embodiments, the subject is selected based on the occurrence of diabetes or hyperlipidemia. In further embodiments the method reduces blood glucose levels and/or lowers blood triglycerides in the subject.

In yet another aspect, provided is methods of preventing or reversing injury to hepatic mitochondria by agents such as fructose, and thus preventing or reversing hepatic steatosis and other conditions associated with liver cell injury and altered fatty acid metabolism, and thus preventing or reversing hepatic fibrosis, or cirrhosis, associated with sustained hepatocyte or stellate cell injury.

In another embodiment, compounds or compositions disclosed herein may be administered in combination with another agent or agents such as niacin, or inhibitors of xanthine oxidase, such as allopurinol, to treat hyperlipidemia or treat liver injury associated with fructose or other agents associated with intracellular fat accumulation, steatosis, fibrosis, or cirrhosis.

In another embodiment, a decrease in the plasma or tissue levels of 11β-hydroxypregnenolone, 11β-hydroxyprogesterone, or metabolites thereof, such as sulfated, glucuronidated, or methylated derivatives, may be employed as a diagnostic test to determine deficiency states of 11β-hydroxypregnenolone or 11β-hydroxyprogesterone.

In another embodiment, an increase in the plasma or tissue levels of 11β-hydroxypregnenolone, 11β-hydroxyprogesterone, or metabolites thereof, such as sulfated, glucoronidated, or methylated derivatives, may be employed as a diagnostic test to determine therapeutic response to administration of 11β-hydroxypregnenolone, 11β-hydroxyprogesterone, or derivatives thereof.

Changes in the plasma or tissue levels of 11β-hydroxypregnenolone or 11β-hydroxyprogesterone or related hydroxysteroids and metabolites thereof may also be employed in conjunction with biomarkers of muscle injury or regeneration, such as myostatin, follistatin, creatine kinase, and others in order to determine deficiency states of the hydroxysteroid pathway or measure therapeutic response to hydroxysteroid-based therapeutics.

In another embodiment, compounds or compositions disclosed herein may be employed as therapeutics, or hormone replacement, in diseases or conditions associated with deficiency states of 11β-hydroxypregnenolone, 11β-hydroxyprogesterone, or metabolites thereof.

In a first aspect, provided is methods for preventing or treating adverse events associated with the use of chemical compositions such as approved medications in which the adverse event is caused by, or associated with, perturbations in mitochondrial number, function, or structure. The methods comprise administering to a subject in need thereof a compound or composition disclosed herein. In certain embodiments the method reduces symptoms of mitochondrial toxicity due to the subject's exposure to chemical compositions that exhibit mitochondrial toxicity.

In certain embodiments, compounds or compositions disclosed herein are administered in combination with one or more chemical compositions which exhibit mitochondrial toxicity. Such chemical compositions include, but are not limited to, those described above in regard to drug-induced mitochondrial dysfunction of the heart, liver, and kidneys.

In certain embodiments, the chemical composition that exhibits mitochondrial toxicity is identified based on the demonstration of one or more biological effects indicative of mitochondrial toxicity by the chemical composition. Such effects include, but are not limited to, abnormal mitochondrial respiration, abnormal oxygen consumption, abnormal extracellular acidification rate, abnormal mitochondrial number, abnormal lactate accumulation, abnormal ATP levels, etc.

In other embodiments, compounds or compositions disclosed herein are administered based on the occurrence of one or more physiological manifestations of mitochondrial toxicity in the subject. Such manifestations include, but are not limited to, elevations in markers known to relate to injury to the heart, liver, and/or kidney. Non-limiting examples include elevated serum liver enzymes, elevated cardiac enzymes, lactic acidosis, elevated blood glucose, elevated serum creatinine, etc.

In certain embodiments, compounds or compositions disclosed herein are administered in combination with one or more chemical compositions which can increase the biological activity of compounds disclosed herein, particularly with respect to effecting mitochondrial biogenesis, promoting muscle regeneration, and enhancing NO availability via the stimulation of the expression and activity of eNOS and nNOS.

In another embodiment, provided is methods for improving muscle structure or function; methods for improving mitochondrial effects associated with exercise; methods for enhancing the capacity for exercise in those limited by age, inactivity, diet, or any of the aforementioned diseases and conditions; methods for enhancing muscle health and function in response to exercise; methods for enhancing muscle health and function in the clinical setting of restricted capacity for exercise, whether due to injury, inactivity, obesity, or any of the aforementioned diseases and conditions; and/or methods to enhance recovery of muscles from vigorous activity or from injury associated with vigorous or sustained activity.

In related aspects, provided is methods of treating a condition involving decreased mitochondrial function in an animal. These methods comprise delivering to the animal one or more compounds or compositions disclosed herein.

In some embodiments, provided is compositions and methods for prophylactic and/or therapeutic treatment of diseases and conditions related to apoptosis and cellular necrosis caused by ischemia. In various aspects described hereinafter, provided is compositions and methods for treatment of acute coronary syndromes, including but not limited to myocardial infarction and angina; acute ischemic events in other organs and tissues, including but not limited to renal injury, renal ischemia and diseases of the aorta and its branches; injuries arising from medical interventions, including but not limited to coronary artery bypass grafting (CABG) procedures and aneurysm repair; and metabolic diseases, including but not limited to diabetes mellitus.

In some embodiments, provided is compositions and methods for prophylactic and/or therapeutic treatment of conditions related to mitochondrial function. In various aspects described hereinafter, provided is a method comprising administering one or more compounds as disclosed herein. Stimulation of mitochondrial function in cells may comprise stimulation of one or more of mitochondrial respiration and mitochondrial biogenesis. The methods and compositions described herein can assist in prevention of impaired mitochondria biogenesis and thus prevention of the consequences of impaired mitochondrial biogenesis in various diseases and conditions, as well as provide for the active therapy of mitochondrial depletion that may have already occurred.

In certain embodiments, compounds or compositions disclosed herein are administered to the subject together with one or more additional drugs useful in the treatment of ischemic or ischemia/reperfusion events. Exemplary additional drugs include one or more compounds independently selected from the group consisting of tetracycline antibiotics (e.g., doxycycline), glycoprotein IIb/IIIa inhibitors (e.g., eptifibatide, tirofiban, abciximab); ADP receptor/P2Y12 inhibitors (e.g., clopidogrel, ticlopidine, prasgurel); prostaglandin analogues (e.g., betaprost, iloprost, treprostinil); COX inhibitors (e.g., asprin, aloxiprin); other antiplatelet drugs (e.g., ditazole, cloricromen, dipyridamole, indobufen, picotamide, triflusal); anticoagulants (e.g., coumarins, 1,3-indandiones); heparins; direct factor Xa inhibitors; direct thrombin (II) inhibitors (e.g., bivalirudin); and vasodilators (e.g., fendoldopam, hydralazine, nesiritide, nicorandil, nicardipine, nitroglycerine, nitroprusside). This list is not meant to be limiting. In a particularly embodiment, a compound or composition disclosed herein is administered together with one or more tetracycline antibiotics such as doxycycline.

In the case of an ischemic event involving the heart, objective measures include increases in one or more cardiac markers (e.g., CK-MB, myoglobin, cardiac troponin I, cardiac troponin T, B-type Natriuretic peptide, NT-proBNP, etc.); changes in serial ECG tracings; and angiographic results.

In the case of an ischemic event involving the kidneys, objective measures include those defined by Bellomo et al., Crit Care. 8(4):R204-12, 2004, which is hereby incorporated by reference in its entirety. This reference proposes the following classifications for stratifying acute kidney injury patients: “Risk”: serum creatinine increased 1.5 fold from baseline OR urine production of <0.5 ml/kg body weight for 6 hours; “Injury”: serum creatinine increased 2.0 fold from baseline OR urine production <0.5 ml/kg for 12 h; “Failure”: serum creatinine increased 3.0 fold from baseline OR creatinine >355 μmol/L (with a rise of >44) or urine output below 0.3 ml/kg for 24 h.

In a related aspect, provided is pharmaceutical compositions for treatment of an acute ischemic or ischemia/reperfusion (IR) event. This composition comprises an effective amount of a compound or composition disclosed herein, and one or more additional drugs useful in the treatment of ischemic or ischemia/reperfusion events. In particularly embodiments, the pharmaceutical composition comprises a compound or composition disclosed herein and one or more tetracycline antibiotics, such as doxycycline. In a further embodiment, the composition is formulated for intravenous delivery.

### Kits

Also provided are articles of manufacture and kits containing any of the compounds and compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing the compounds and compositions described herein and instructions for use. The kits may contain instructions for use in the treatment of any disease provided herein in a subject in need thereof. A kit may additionally contain any materials or equipment that may be used in the administration of the compounds and compositions, such as vials, syringes, or IV bags. A kit may also contain sterile packaging.

### ENUMERATED EMBODIMENTS

1. A compound of Formula (I):

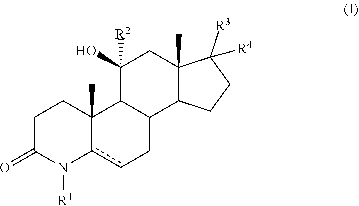

or a pharmaceutically acceptable salt, stereoisomer or tautomer thereof, wherein:
R¹ is H or C₁₋₆ alkyl;
R² is H, D, or C₁₋₆ alkyl;
R³ is C₁₋₆ alkyl, C₁₋₆ alkoxy, C(O)R⁵, NC(O)R⁵, NC(O)NR₆R₇, C(O)OR′, NHR⁵, or 5-membered heterocyclyl, wherein C₁₋₆ alkyl is optionally substituted with one or more of OH or D, and 5-membered heterocyclyl is optionally substituted with C₁₋₆ alkyl;
R⁴ is H;
or R³ and R⁴ taken together with the carbon atom to which they are attached form a 5-membered heterocycle;
R⁵ is H, C₁₋₆ alkyl, C₃₋₁₀ cycloalkyl, SO₂R⁶, NR⁶R⁷ or 5-6 membered heterocyclyl optionally substituted with one or more C₁₋₆ alkyl;
R⁶ and R⁷ are each independently H, C₃₋₆ cycloalkyl, or C₁₋₆ alkyl optionally substituted with C₃₋₆ cycloalkyl; and
the dashed line represents an optional double bond.
2. The compound of embodiment 1, wherein the compound is of Formula (Ia):

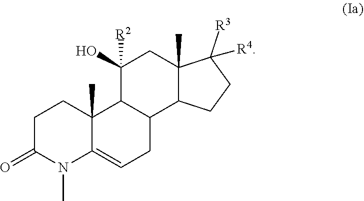

3. The compound of embodiment 2, wherein R² is H.
4. The compound of embodiment 2 or 3, wherein R⁴ is H.
5. The compound of embodiment 1, wherein the compound is of Formula (Ib):

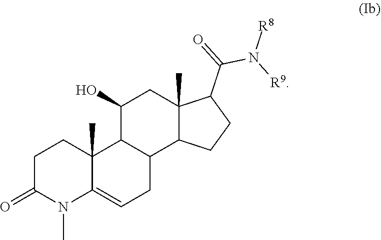

6. The compound of embodiment 5, wherein R⁹ is H.
7. The compound of embodiment 1, wherein the compound is of Formula (Ic):

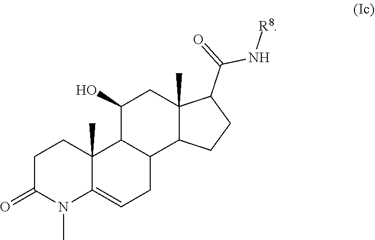

8. The compound of embodiment 7, wherein R⁸ is C₁₋₆ alkyl.
9. The compound of embodiment 8, wherein R⁸ is methyl.
10. The compound of embodiment 8, wherein R⁸ is isohexyl.
11. The compound of embodiment 7, wherein R⁸ is C₃₋₁₀ cycloalkyl.
12. The compound of embodiment 7, wherein R⁸ is C₁₋₆ alkyl substituted with C₃₋₁₀ cycloalkyl.
13. The compound of embodiment 1, wherein the compound is of Formula (Id):

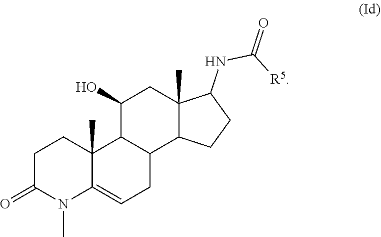

14. The compound of embodiment 13, wherein R⁵ is C₃₋₁₀ cycloalkyl.
15. The compound of embodiment 14, wherein R⁵ is cyclopropyl.
16. The compound of embodiment 13, wherein the compound is of Formula (Ie):

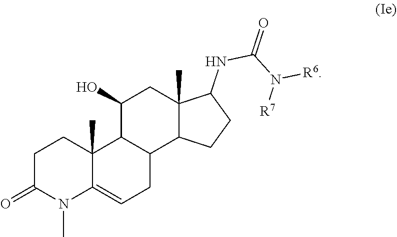

17. The compound of embodiment 16, wherein R⁶ is C₃₋₁₀ cycloalkyl or C₁₋₆ alkyl optionally substituted with C₃₋₁₀ cycloalkyl.
18. The compound of embodiment 16 or 17, wherein R⁷ is H.
19. A compound of Formula (II):

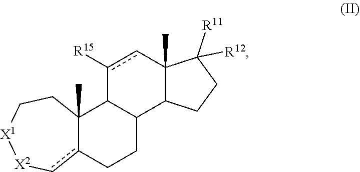

or a pharmaceutically acceptable salt, stereoisomer or tautomer thereof, wherein:
one of X¹ and X² is NR¹⁰, and the other is C═O,
R¹⁰ is H or C₁₋₆ alkyl;
R¹¹ is C₁₋₆ alkoxy, C(O)OR¹³, C(O)NR¹³R¹⁴, C(O)R¹³, NR¹³R¹⁴, NC(O)R¹³, or C₁₋₆ alkyl optionally substituted with one or more R¹³;
R¹² is H or OH; or
R¹¹ and R¹² together form oxo;
R¹³ and R¹⁴ are each independently H, C₁₋₆ alkyl, C₃₋₆ cycloalkyl, or NH₂;
R¹⁵ is H or OH; and each dashed line represents an optional double bond.
20. The compound of embodiment 19, wherein R⁵ is OH.
21. The compound of embodiment 19, wherein the compound is of Formula (IIa):

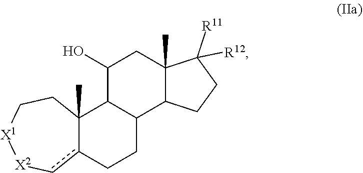

22. The compound of embodiment 21, wherein R¹² is H.
23. The compound of embodiment 21 or 22, wherein X¹ is NR¹⁰ and X² is C═O.
24. The compound of embodiment 21, wherein the compound is of Formula (IIb):

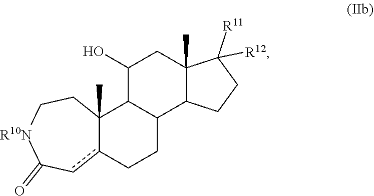

25. The compound of embodiment 24, wherein R¹⁰ is C₁₋₆ alkyl.
26. The compound of embodiment 25, wherein R¹⁰ is methyl.
27. The compound of embodiment 24, wherein the compound is of Formula (IIc):

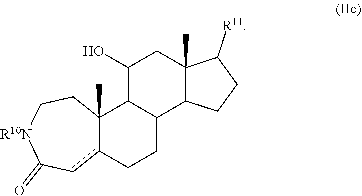

28. The compound of embodiment 27, wherein R¹⁰ is methyl.
29. The compound of embodiment 27 or 28, wherein R¹¹ is C₁₋₆ alkoxy.
30. The compound of embodiment 27 or 28, wherein R¹¹ is NH₂.
31. The compound of embodiment 27, wherein the compound is of Formula (Id):

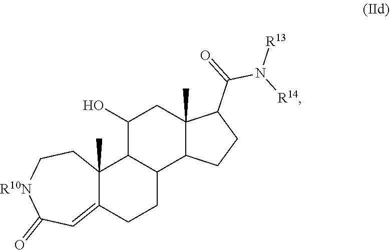

32. The compound of embodiment 31, wherein R¹⁰ is methyl.
33. The compound of embodiment 31 or 32, wherein R¹³ and R¹⁴ are each independently C₁₋₆ alkyl.
34. The compound of embodiment 33, wherein R¹³ and R¹⁴ are each methyl.
35. The compound of embodiment 27, wherein the compound is of Formula (IIe):

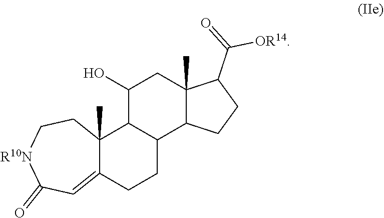

36. The compound of embodiment 35, wherein R¹⁰ is methyl.
37. The compound of embodiment 35 or 36, wherein R¹⁴ is hydrogen or methyl.
38. The compound of embodiment 24, wherein the compound is of Formula (IIg):

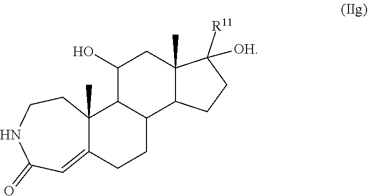

39. The compound of embodiment 38, wherein R¹¹ is C(O)NHR¹³, wherein R¹³ is C₁₋₆ alkyl or C₃₋₆ cycloalkyl.
40. The compound of embodiment 21, wherein the compound is of Formula (IIh):

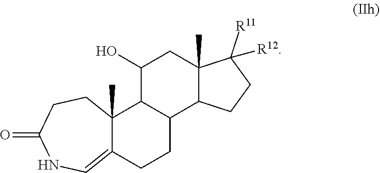

41. The compound of embodiment 40, wherein R¹¹ is C(O)NHR¹³, wherein R¹³ is C₁₋₆ alkyl or C₃₋₆ cycloalkyl.
42. The compound of embodiment 41, wherein R¹³ is isopropyl or cyclobutyl.
43. A compound of Formula (III):

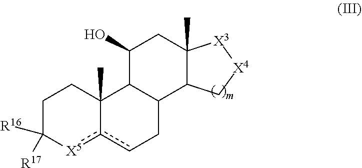

wherein
R¹⁶ and R¹⁷ together form oxo, or
R¹⁶ and R¹⁷ together with the carbon atom to which they are attached form a 5-membered heterocycle;
one of X³ and X⁴ is CH₂ or C═O, and the other is NR¹⁸;
X⁵ is CH₂, CH, or NR¹⁹.
R¹⁸ is H, C₁₋₆ alkyl, or C(O)R¹⁹.
each R¹⁹ is independently H or C₁₋₆ alkyl;
m is 1 or 2; and
the dashed lines represent optional double bonds, wherein the number of dashed lines representing double bonds is zero or one.
44. The compound of embodiment 43, wherein the compound is of Formula (IIIa):

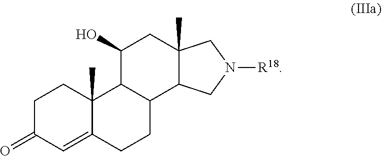

45. The compound of embodiment 43, wherein the compound is of Formula (IIIb):

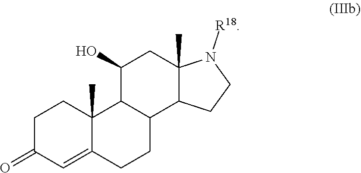

46. The compound of embodiment 43, wherein the compound is of Formula (IIIc):

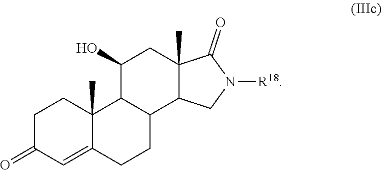

47. The compound of embodiment 43, wherein the compound is of Formula (IIId):

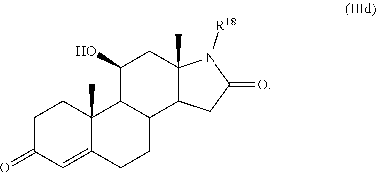

48. The compound of embodiment 43, wherein the compound is of Formula (IIIe):

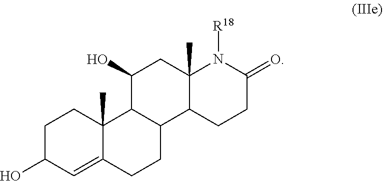

49. A compound selected from the group consisting of:

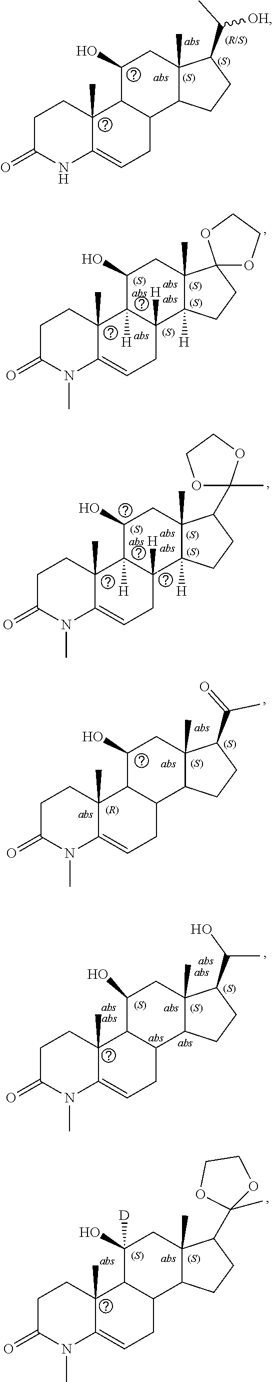

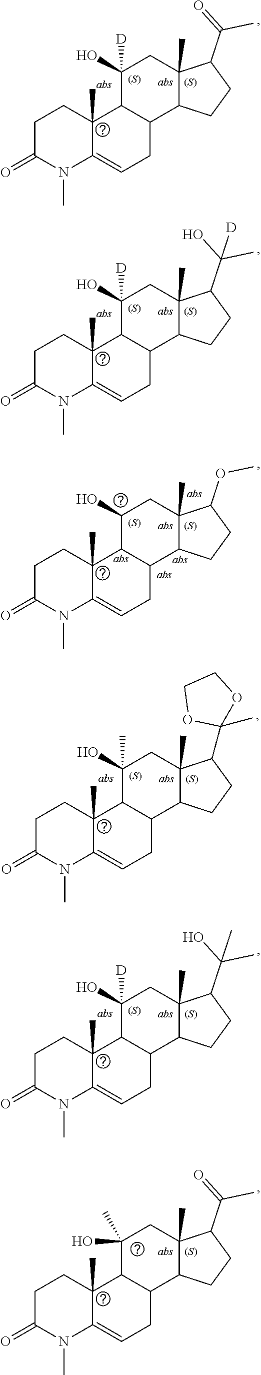

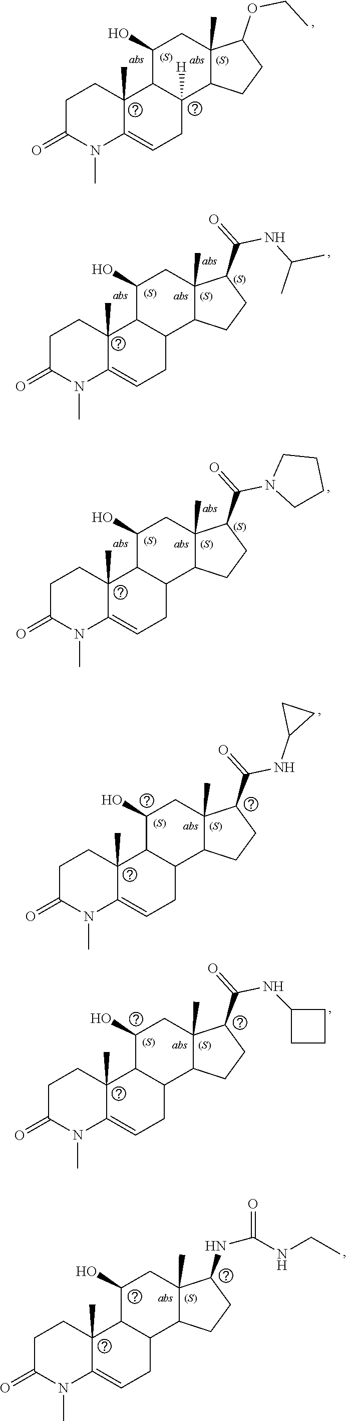

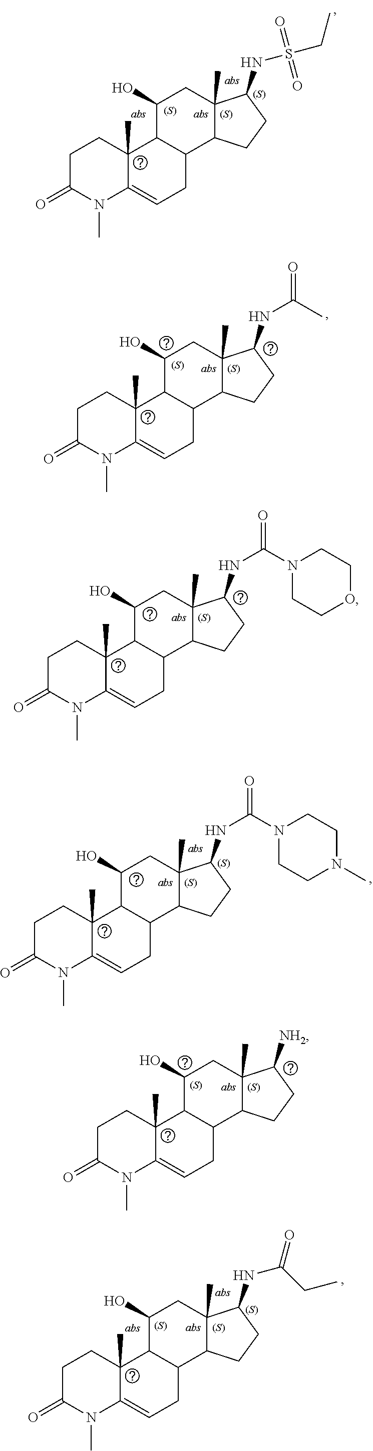

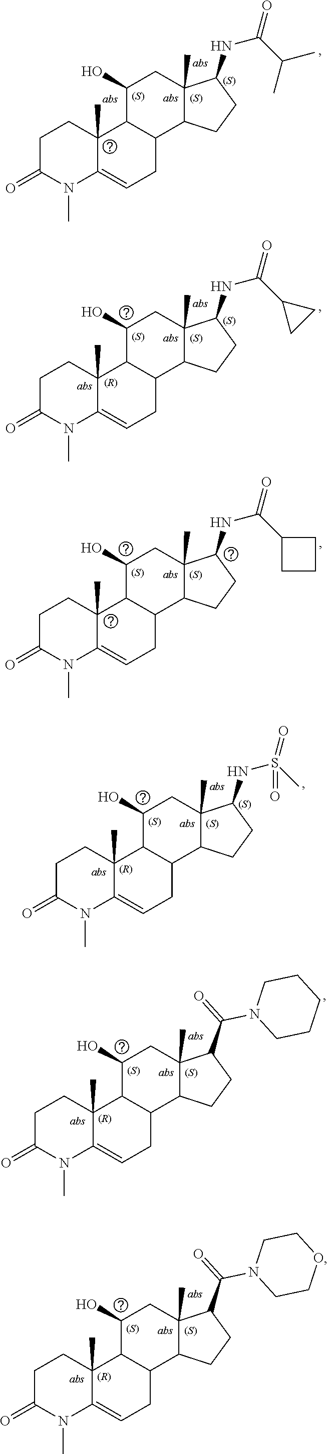

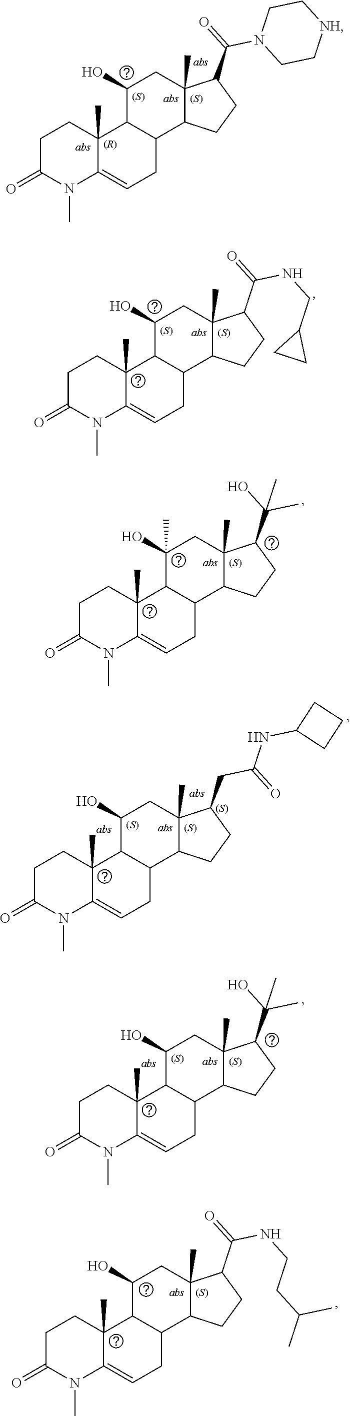

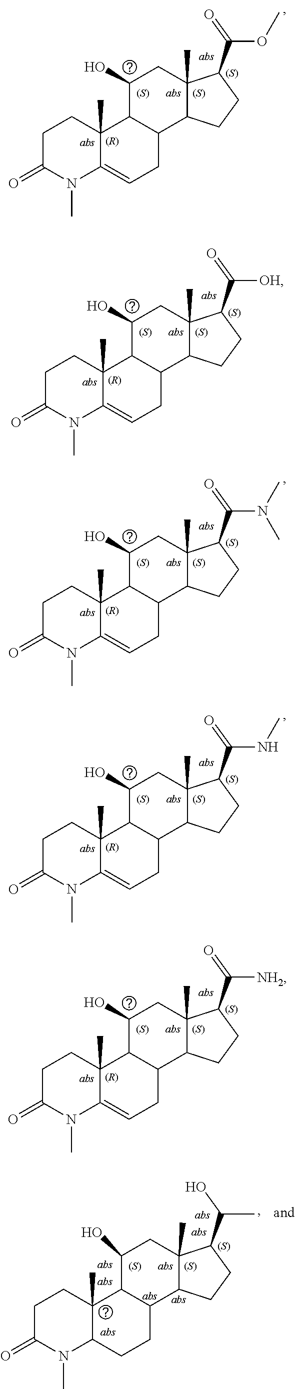

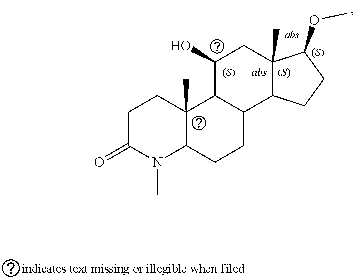

or a pharmaceutically acceptable salt, stereoisomer or tautomer thereof.
50. A compound selected from the group consisting of:

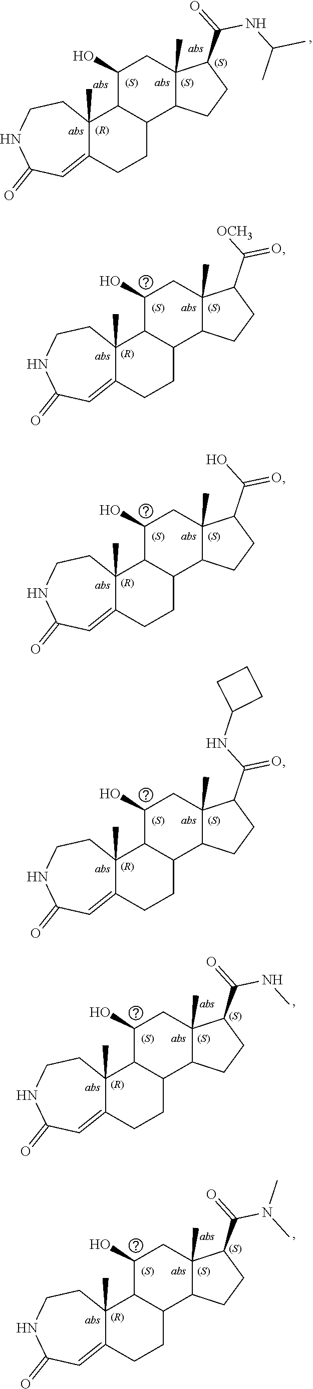

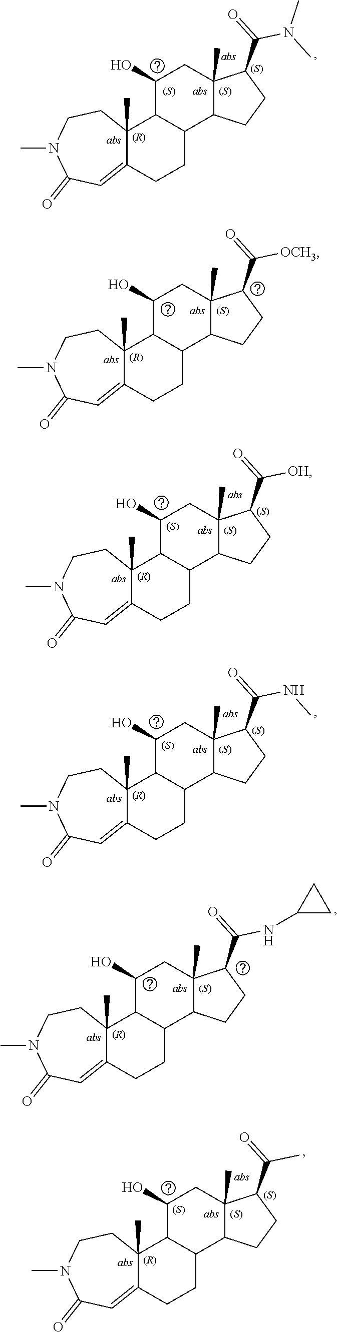

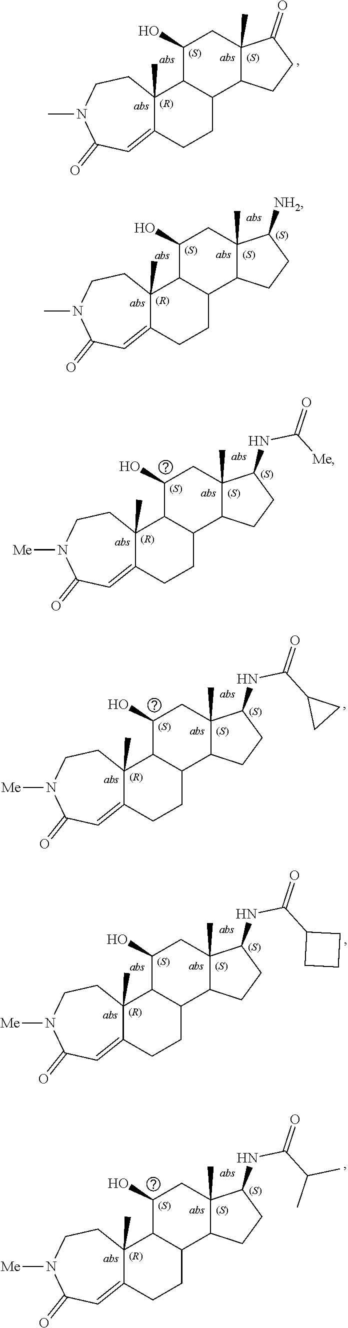

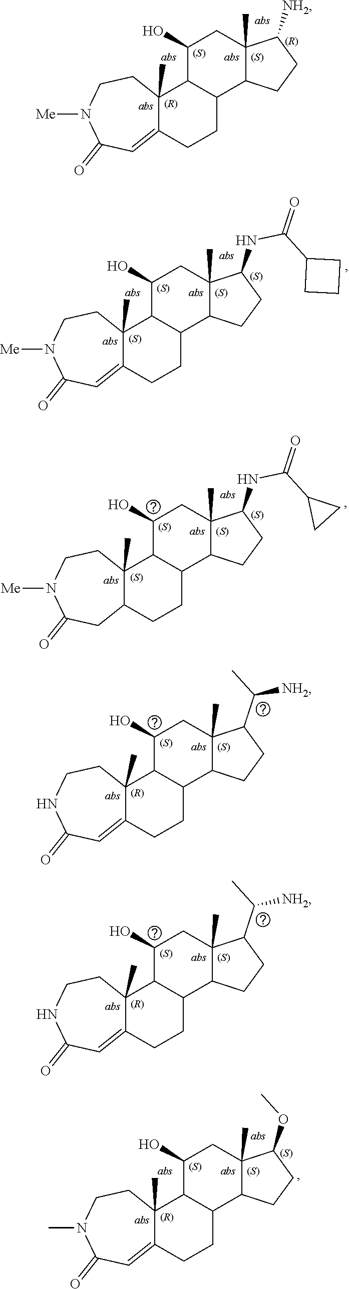

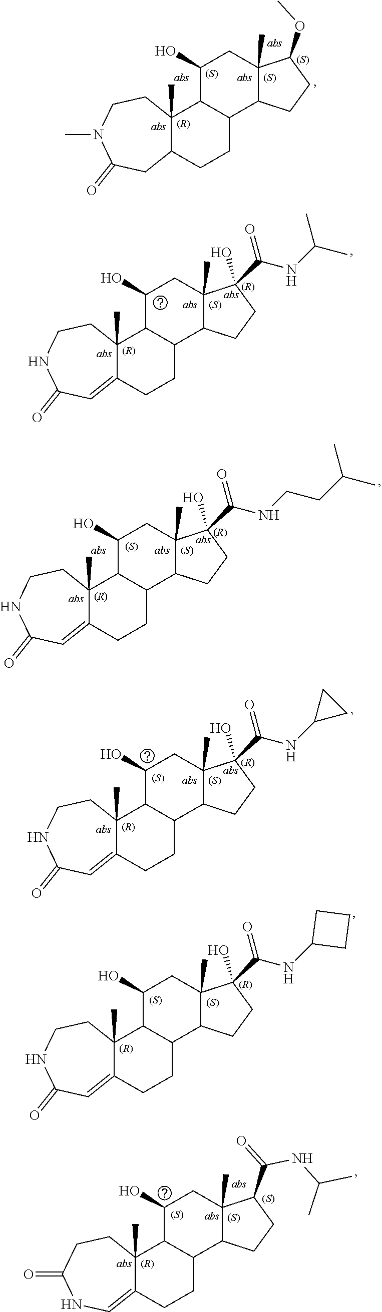

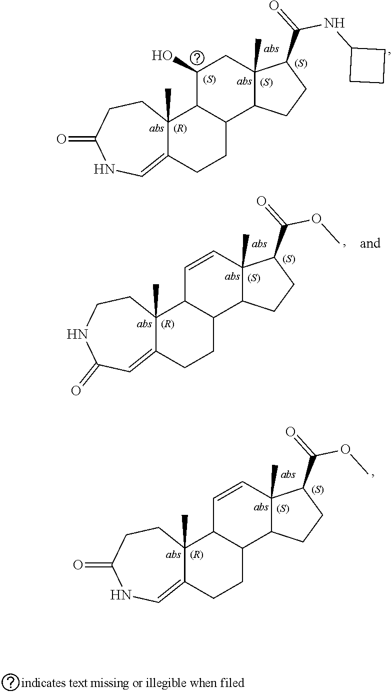

or
a pharmaceutically acceptable salt, stereoisomer or tautomer thereof.
51. A compound selected from the group consisting of

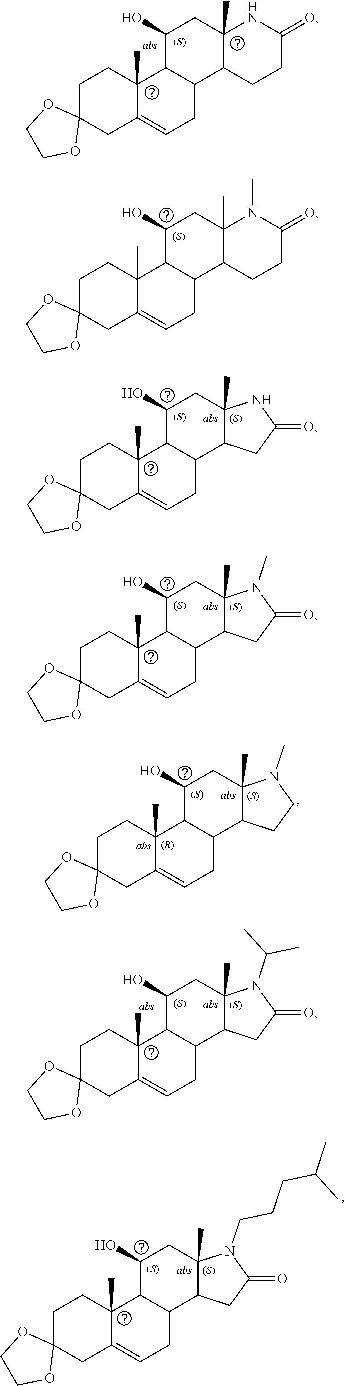

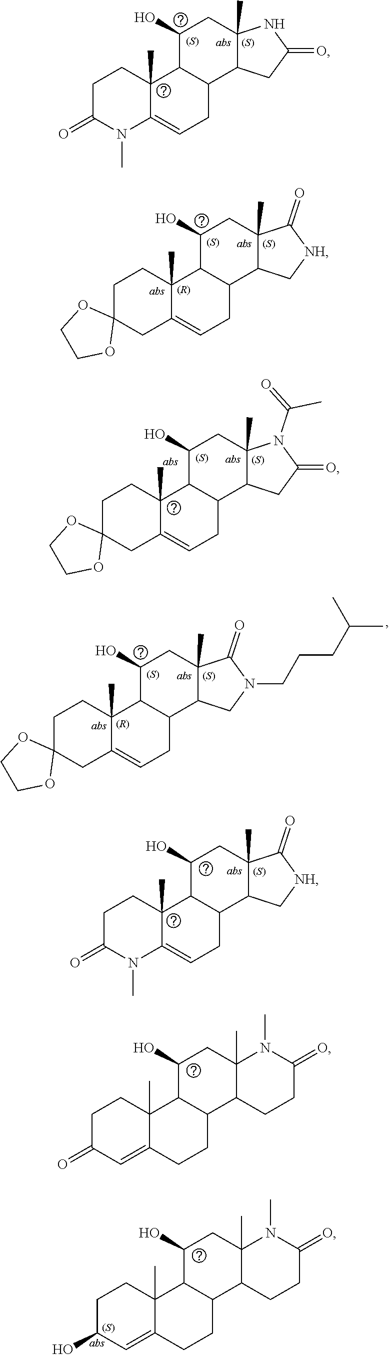

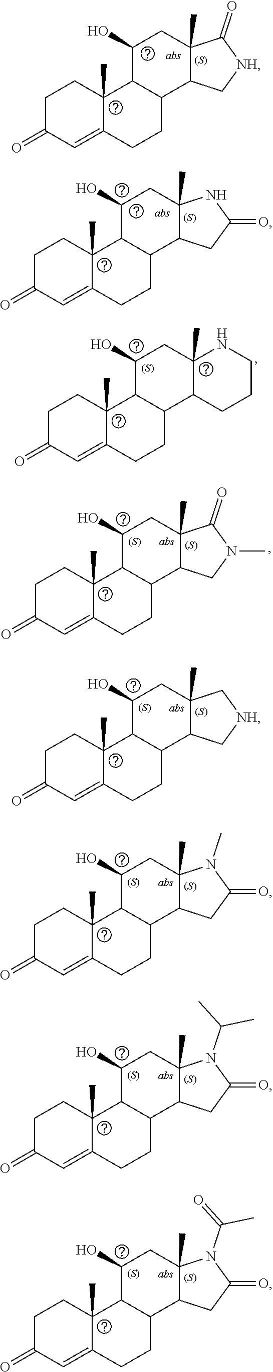

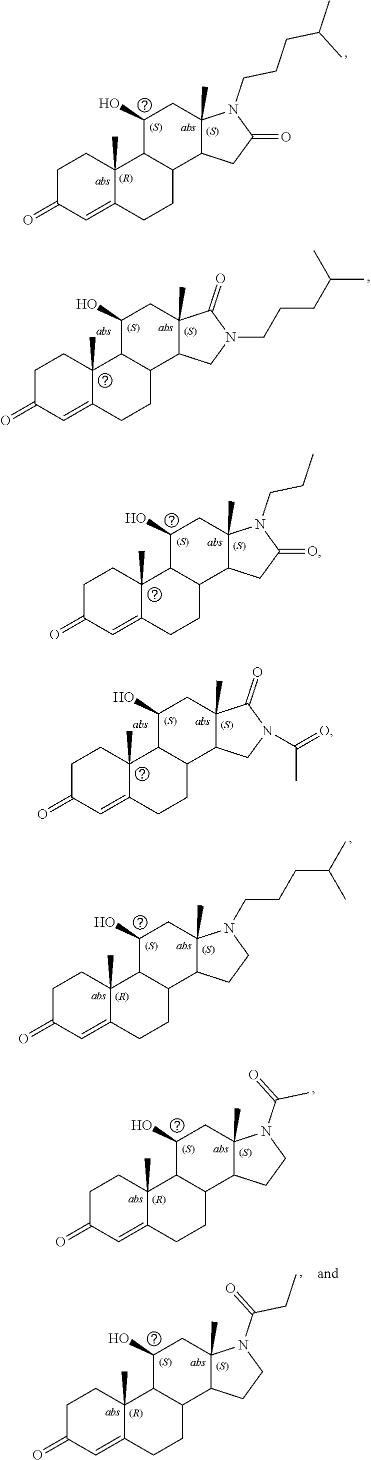

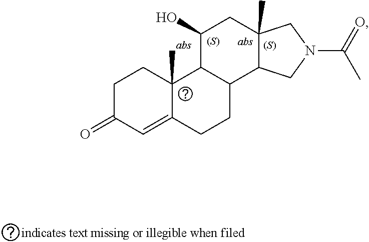

or a pharmaceutically acceptable salt, isomer, or tautomer thereof.
52. A compound selected from the group consisting of

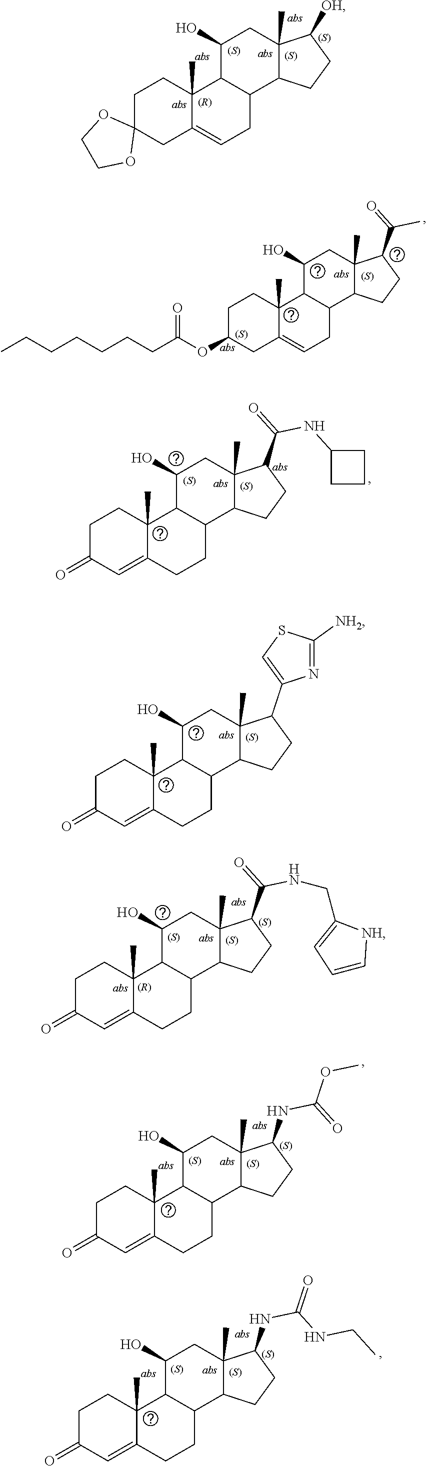

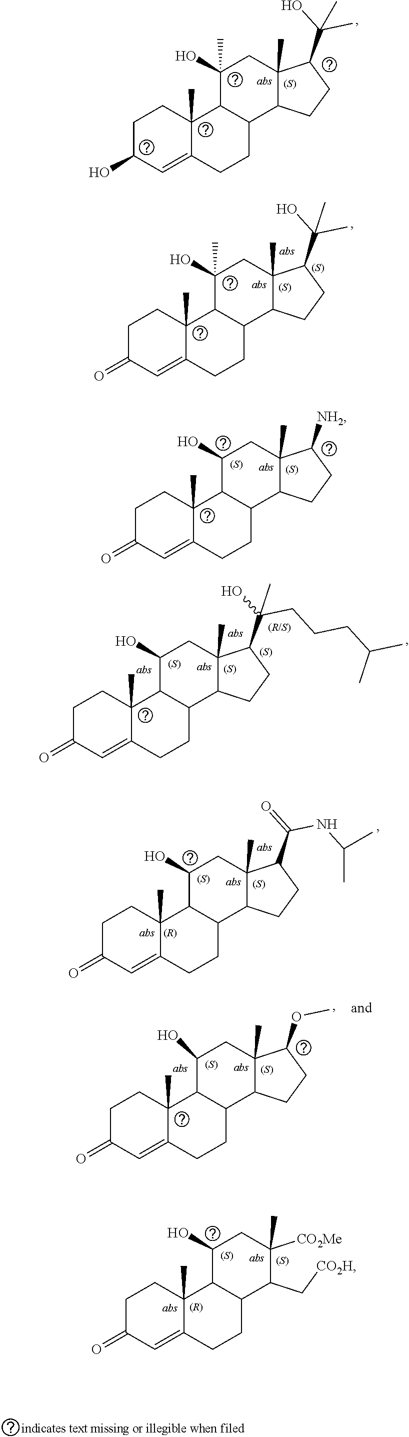

or a pharmaceutically acceptable salt, isomer, or tautomer thereof.
53. A pharmaceutical composition comprising a compound of any one of embodiments 1-52, a pharmaceutically acceptable salt thereof, and a pharmaceutically acceptable carrier.
54. A compound, or a pharmaceutically acceptable salt thereof, of any one of embodiments 1-52 for use as therapeutically active substance.
55. Use of a compound, or a pharmaceutically acceptable salt thereof, of any one of embodiments 1-52 or a pharmaceutical composition of embodiment 53 for the preparation of a medicament for the treatment of a disease, disorder or condition.

Certain specific aspects and embodiments will be explained in more detail with reference to the following examples, which are provided only for purposes of illustration and should not be construed as limiting the scope in any manner.

### EXAMPLES

### List of Abbreviations

AICAR 5-aminoimidazole-4-carboxamide-1-β-D-ribofuranoside
AMPK AMP activated protein kinase
BLQ Below the limit of quantitation
BSA Bovine Serum Albumin
CMC Carboxy methyl Cellulose
DMEM Dulbecco's Modified Eagle Medium
ELISA Enzyme-linked immunosorbent assay
ESI-MS Electrospray ionization mass spectrometry
FBS Fetal bovine serum
HPLC High performance liquid chromatography
HRP Horseradish peroxidase
IP Intra-peritoneal
IVC Individually ventilated caging
LCMS Liquid chromatography-mass spectrometry
LPS Lipopolysaccharide
NMP N-methyl-2-pyrrolidone
PBS Phosphate buffered saline
PGC1α PPARG coactivator 1 alpha
PO Per Oral
PPARGC1A Peroxisome proliferator-activated receptor gamma coactivator 1-alpha
RIPA Radioimmunoprecipitation assay
RPM Rotation per minute
RT Room temperature
TMB Tumor mutational burden
μl Micro litre
° C. Degree Celsius
BID Twice a day dosing

### Example 1: Synthesis of Compound 26

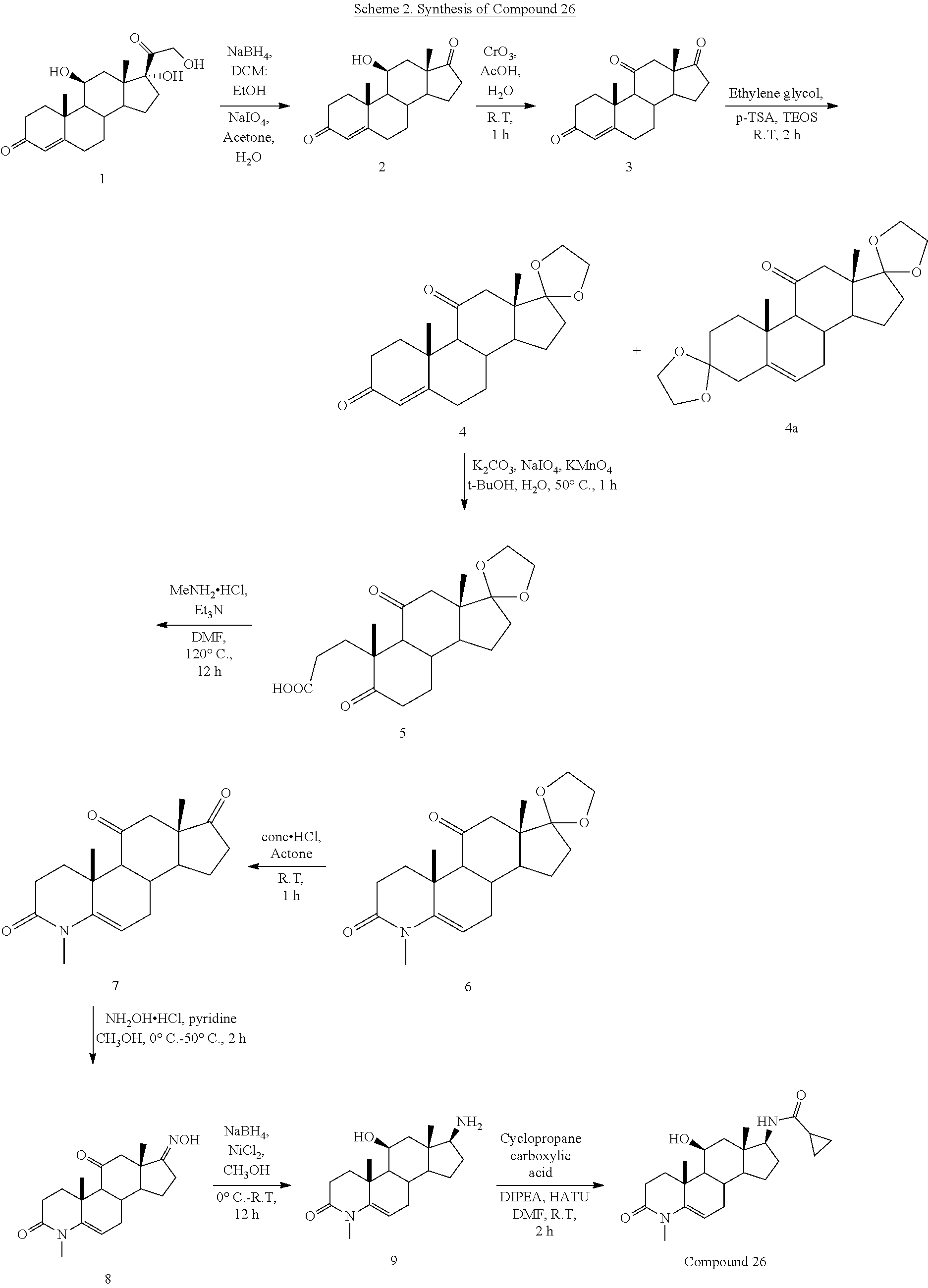

### 1. Synthesis of (10R,11S,13S)-11-hydroxy-10,13-dimethyl-1,6,7,8,9,10,11,12,13,14,15,16-dodecahydro-3H-cyclopenta[ja]phenanthrene-3,17(2H)-dione (2)

A round-bottomed flask was charged with compound 1 (50 g, 0.17 mol) in DCM and EtOH (15:1, 320 mL). This was followed by the slowly addition of NaBH₄ (2.52 g, 0.07 mol) to the reaction mixture at 0° C. After addition of all NaBH₄ gave stirring at R.T for 1 h. Monitor the reaction by TLC. After consuming of all starting material, acetone (200 mL) was added to this reaction mixture followed by the slowly addition of NaIO₄ (73.1 g, 0.34 mol, dissolve in 100 mL water), gave continue stirring at the same temperature for another 1 h. Monitor the reaction by TLC. After completion the reaction remove extra solvent under reduce pressure. Dilute this reaction mixture with dis. Water, aqueous layer extracted with dichloromethane, organic layer dry over anhydrous sodium sulphate and concentrate under reduced pressure offered desired compound 2 as a white solid (40.1 g, 78.1% yield). This compound was used as such for the next step.

### 2. Synthesis of (10R,13S)-10,13-dimethyl-6,7,8,9,10,12,13,14,15,16-decahydro-1H-cyclopenta[a]phenanthrene-3,11,17(2H)-trione (3)

A round-bottomed flask was charged with compound 2 (43.0 g, 142.8 mmol) in acetic acid (280 mL). This was followed by the slowly addition of CrO₃ (23.6 g, 157.1 mmol, dissolve in 20 mL) to the reaction mixture. Give stirring at R.T for 1 h. Monitor the reaction by TLC. After completion of the reaction, remove the extra solvent under reduce pressure. Black residues were obtained, sat. Solution NaHCO₃ was added slowly to this crude material at 0° C., white precipitates were formed, filter these precipitate through Buchner funnel, dried well and azeotrope with toluene to offered desired compound 3 (32.8 g, 76.45% yield). This compound was used as such for the next step.

### 3. Synthesis of (10R,13S)-10,13-dimethyl-1,2,6,7,8,9,10,12,13,14,15,16-dodecahydrospiro[cyclopenta[a]phenanthrene-17,2′-[1,3]dioxolane]-3,11-dione & (10′R,13'S)-10′,13′-dimethyl-1′,2′,4′,7′,8′,9′,10′,12′,13′,14′,15′,16′-dodecahydro-11′H-dispiro[1,3]dioxolane-2,3′-cyclopenta[a]phenanthrene-17′,2″-[1,3]dioxolan]-11′-one (4 & 4a)

A round-bottomed flask was charged with compound 3 (32.0 g, 106.3 mmol) in ethylene glycol (400 mL). This was followed by the slowly addition of p-TSA (1.2 g, 6.4 mmol) and tetraethyl orthosilicate (4.5 mL, 212.6 mmol) to the reaction mixture at R.T under inert atmosphere. Monitor the reaction by TLC. After completion of the reaction, quench the reaction with sat. Solution NaHCO₃, extracted with dichloromethane. Organic layer were dried over sodium sulphate, concentrated and purified by column offered us two products (4 and 4a). First compound i.e. desired compound (mono acetal) 4 was found (14.0 g, 38.24% yield) while second compound 4a i.e. di acetal was found (16.0 g, 38.7%). Compound 4 used as such for the next the step.

### 4. Synthesis of 3-((3aS,6R)-3a,6-dimethyl-5,7-dioxododecahydrospiro[cyclopenta[a]naphthalene-3,2′-[1,3]dioxolan]-6-yl)propanoic acid (5)

A round-bottomed flask was charged with compound 4 (7.0 g, 20.28 mmol) in t-BuOH (140 mL). This was followed by the addition of K2CO3 (5.04 g, 36.5 mmol, dissolve in 70 mL), then KMnO4 (0.48 g, 3.04 mmol, dissolve in 140 mL) and NaIO₄ (21.7 g, 101.4 mmol, dissolve in 140 mL) sequentially to the reaction mixture slowly at R.T. Reaction mixture was transferred to 50° C., gave stirring for 1 h. Monitor the reaction by TLC. After completion of the reaction, evaporate t-BuOH then add citric acid till pH gets acidified. Aqueous layer were extracted with dichloromethane. Organic layer were concentrated to obtained desired white solid compound 5 (6.0 g, 81.1% yield). ESI-MS (m/z) Calcd for C₂₀H₂₈O₆: 364.44 Found 363.3 (M)-.

### 5. Synthesis of (4aR,6aS)-1,4a,6a-trimethyl-1,3,4,4a,4b,6,6a,8,9,9a,9b,10-dodecahydrospiro[indeno[5,4-f]quinoline-7,2′-[1,3]dioxolane]-2,5-dione (6)

A round-bottomed flask was charged with compound 5 (6.0 g, 16.48 mmol) in DMF (35.0 mL). This was followed by the addition of Et₃N (13.7 mL, 98.9 mmol) and CH₃NH₂·HCl (4.3 g, 64.2 mmol) to the reaction mixture at R.T under inert atmosphere, reaction mixture was transferred at 120° C., gave stirring for 12 h. Monitor the reaction by TLC. After completion of the reaction, cool the reaction mixture and add chilled water to it, aqueous layer were extracted with ethyl acetate. Organic layer were concentrated, obtained residues were purified by column chromatography to yield compound 6 (5.0 g, 84.5% yield). ESI-MS (m/z) Calcd for C₂₁H₂₉NO₄: 359.47 Found 360.4 (M+H)+.

### 6. Synthesis of (4aR,6aS)-1,4a,6a-trimethyl-3,4,4a,6,6a,8,9,9a,9b,10-decahydro-1H-indeno[5,4-f]quinoline-2,5,7(4bH)-trione (7)

A round-bottom flask was charged with compound 6 (5.0 g, 13.92 mmol) in acetone (100 mL). HCl (1.0 N, 7.0 mL) was added to this solution, gave stirring at room temperature for 1 h. Monitor the reaction by TLC. After completion of the reaction, remove extra solvent. Sat. solution of NaHCO₃ was added to it at 0° C., extracted with dichloromethane. Organic layer were concentrated to obtained desire product 7 (4.2 g, 95.45% yield). Compound 7 was used as such for the next step.

### 7. Synthesis of (4aR,6aS,Z)-7-(hydroxyimino)-1,4a,6a-trimethyl-3,4,4a,4b,6,6a,7,8,9,9a,9b,10-dodecahydro-1H-indeno[5,4-f]quinoline-2,5-dione (8)

A round-bottomed flask was charged with compound 7 (4.2 g, 12.8 mmol) in CH₃OH (100 mL) under a nitrogen atmosphere. This was followed by the addition of pyridine (3.1 mL, 38.4 mmol) and NH₂OH·HCl (1.0 g, 14.2 mmol) to the reaction mixture at 0° C. under inert atmosphere, reaction mixture was transferred to 50° C., gave stirring for 2 h. After completion of the reaction, remove extra methanol by using rota-evaporator then gave washing with dis. water and ethyl acetate, aqueous layer were extracted with ethyl acetate. Organic layer were concentrated, obtained residue was purified by column chromatography to yield compound 8 (4.2 g, 97.7% yield). ESI-MS (m/z) Calcd for C₁₉H₂₆N₂O₃: 330.43 Found 331.2 (M+H)+.

### 8. Synthesis of (4aR,5S,6aS,7S)-7-amino-5-hydroxy-1,4a,6a-trimethyl-1,3,4,4a,4b,5,6,6a,7,8,9,9a,9b,10-tetradecahydro-2H-indeno[5,4-f]quinolin-2-one (9)

A round-bottomed flask was charged with compound 8 (4.0 g, 12.1 mmol) in CH₃OH (100 mL) under a nitrogen atmosphere. This was followed by the addition of NiCl₂ (3.1 g, 24.2 mmol) and portion wise NaBH₄ (6.9 g, 181.7 mmol) to the reaction mixture at 0° C., reaction mixture was stirred at the same temperature under inert atmosphere for another 12 h. After completion of the reaction, reaction mixture pass through celite and gave washing with ethyl acetate, organic part was concentrated, obtained residue was purified by column chromatography to yield compound 9 (1.7 g, 43.6% yield). Compound 9 was used as such for the next step.

### 9. Synthesis of N-((4aR,5S,6aS,7S)-5-hydroxy-1,4a,6a-trimethyl-2-oxo-2,3,4,4a,4b,5,6,6a,7,8,9,9a,9b,10-tetradecahydro-1H-indeno[5,4-f]quinolin-7-yl)cyclopropanecarboxamide (Compound 26)

A round-bottomed flask was charged with cyclopropanecarboxylic acid (0.183 g, 2.14 mmol) in DMF (4.0 mL) under a nitrogen atmosphere. This was followed by the addition of DIPEA (1.9 mL, 10.7 mmol) and HATU (1.7 g, 4.28 mmol) to the reaction mixture at 0° C. After 15 min compound 9 (0.68 g, 2.14 mmol) was added and the reaction mixture was stirred at room temperature under inert atmosphere for another 2 h. After completion of the reaction, Reaction mixture was poured in ice, precipitates were formed which were filtered through buchner funnel, wash these precipitates with diethyl ether, and dry under high vacuo to furnished our desired product obtained residue purified by column chromatography to yield Compound 26, a brown solid (0.46 g, 55.6% yield). 1H-NMR (300 MHz, DMSO-d₆) δ: 7.71 (d, J=8.7, 1H), 4.86 (s, 1H), 4.86 (s, 1H), 3.67 (q, J=9.3 Hz, 1H), 3.3 (s, 1H), 2.98 (s, 3H), 2.51-2.28 (m, 5H), 1.87-1.74 (s, 4H), 1.64-1.61 (m, 2H), 1.48-1.44 (m, 2H), 1.27-1.24 (m, 3H), 1.21 (s, 3H), 1.13 (m, 1H), 0.91 (s, 3H), 0.63-0.59 (in, 4H). ESI-MS (m/z) Calcd for C₂₃H₃₄N₂O₃: 386.54 Found 387.2 (M+H)+, HPLC purity: 98.5%.

### Example 2: Synthesis of Compound 36

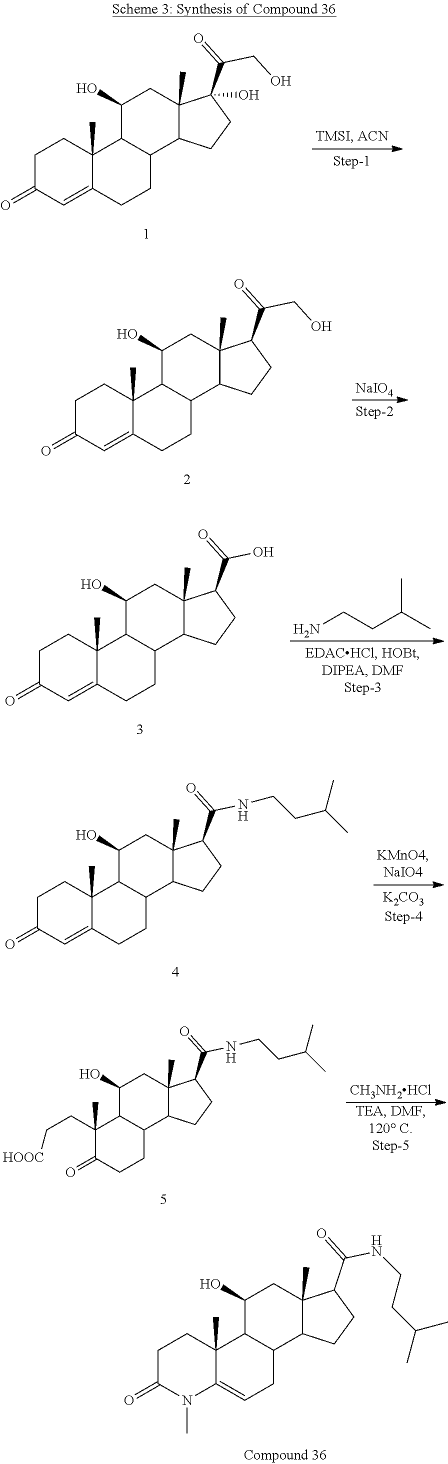

### 1. Synthesis of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one (2)

To a stirred solution of (10R,11S,13S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one, 1 (30.0 g, 82.87 mmol, 1.0 eq) in acetonitrile (500 mL), was added TMSI (59.2 mL, 414.36 mmoL, 5.0 eq) and stirred at room temperature for 2 hrs. After completion of reaction, the reaction mixture was poured into saturated sodium bisulphite solution and removed the organics under reduced pressure. Then, obtained crude was washed with EtOAc and, separated organic layer was dried over sodium sulfate and concentrated to obtain (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one 2 (25.0 g, yellow solid, 87.4%). ESI-MS (m/z) Calcd for C₂₁H₃₀O₄: 346.21 Found 347.4 [M+H]+.

### 2. Synthesis of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (3)

To a stirred suspension of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one, 2 (25.0 g, 72.25 mmol, 1.0 eq) in acetone: water (700 mL:800 mL), NaIO₄ (62.0 g, 289.71 mmoL, 4.0 eq) was added. The reaction was stirred at room temperature for 2 hrs. After completion of the reaction, solvent was removed under reduced pressure. Then, obtained aqueous was acidified using 2N HCl and extracted with dichloromethane. The organic layer was dried over anhyd. sodium sulfate, filtered and concentrated in vacuo to obtain the crude product which was subjected to column chromatography to obtain (silica gel: 100-200 mesh; 1.5% MeOH:DCM) to obtain (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid 3 (22.0 g, yellow solid, 91.9%). ESI-MS (m/z) Calcd for C₂₀H₂₈O₄: 332.20 Found 333.2 [M+H]+.

### 3. Synthesis of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (4)

To a stirred solution of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid, 3 (4.00 g, 12.04 mmol, 1.0 eq) dissolved in DMF (mL) was added HOBt (2.72 g, 18.06 mmol, 1.5 eq), EDC·HCl (3.46 g, 18.06 mmol, 1.5 eq) and triethylamine (8.3 mL, 60.20 mmol, 5.0 eq) at 0° C., followed by addition of 3-methylbutan-1-amine, (1.15 g, 13.25 mmol, 1.1 eq). The reaction mixture was slowly warmed to room temperature and stirred for 16 hours. After completion of the reaction, water was added to the reaction mixture and compound was extracted with ethyl acetate and the organic layer was washed with aq. NaHCO₃ followed by brine solution. The organic layer was dried over anhyd. sodium sulfate, filtered and concentrated in vacuo to obtain the crude product which was subjected to column chromatography to obtain (silica gel: 100-200 mesh; 2% MeOH:DCM) to obtain (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid, 4 (2.35 g, brown solid, 48.65% Yield). ESI-MS (m/z) Calcd for C₂₅H₃₉NO₃: 401.59 Found 402.3 [M+H]+.

### 4. Synthesis of 3-((3aS,5S,6R)-5-hydroxy-3-(isopentylcarbamoyl)-3a,6-dimethyl-7-oxododecahydro-1H-cyclopenta[a]naphthalen-6-yl)propanoic acid (5)

To a stirred solution of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid, 4 (2.3 g, 5.73 mmol, 1.0 eq) in t-BuOH (70 mL) was added a solution of K₂CO₃ (1.42 g, 10.31 mmol, 1.8 eq) in water (10 mL). The reaction mixture was warmed to 50° C. and a solution of KMnO₄ (0.135 g, 0.854 mmol, 0.15 eq) and NaIO₄ (6.12 g, 28.65 mmol, 5.0 eq) in water (75 mL) was added dropwise. Then the reaction mixture was stirred at 50° C. for 1 hour. After completion of the reaction, the reaction mixture was concentrated in-vacuo and the residue was diluted with H2O followed by acidification using citric acid solution. The product was extracted using ethyl acetate and the combined organic layer was dried over anhyd. sodium sulfate solution, filtered and concentrated in vacuo to obtain the crude product 3-((3aS,5S,6R)-5-hydroxy-3-(isopentylcarbamoyl)-3a,6-dimethyl-7-oxododecahydro-1H-cyclopenta[a]naphthalen-6-yl)propanoic acid, 5 (1.70 g, 70.53%) which was used for next step without further purification. ESI-MS (m/z) Calcd for C₂₄H₃₉NO₅: 421.58 Found 422.2 [M+H]+.

### 5. Synthesis of (4aR,5S,6aS)-5-hydroxy-N-isopentyl-1,4a,6a-trimethyl-2-oxo-2,3,4,4a,4b,5,6,6a,7,8,9,9a,9b,10-tetradecahydro-1H-indeno[5,4-f]quinoline-7-carboxamide (Compound 36)

In a seal tube, a solution of 3-((3aS,5S,6R)-5-hydroxy-3-(isopentylcarbamoyl)-3a,6-dimethyl-7-oxododecahydro-1H-cyclopenta[a]naphthalen-6-yl)propanoic acid, 5 (1.68 g, 3.99 mmol, 1.0 eq) in DMF (30 mL) was taken and ethanamine hydrochloride (2.15 g, 31.92 mmol, 8.0 eq) and TEA (5.5 mL, 39.90 mmol, 10.0 eq) was added to the reaction mixture at room temperature. Then the seal tube was closed and reaction mixture was heated at 120° C. for 18 hours. After completion of the reaction, reaction mixture was diluted with water and extracted using ethyl acetate. The combined organic layer was dried over anhyd. sodium sulfate solution, filtered and concentrated in vacuo to obtain the crude product which was subjected to column chromatography to obtain (silica gel: 100-200 mesh; 2% MeOH:DCM) to obtain (4aR,5S,6aS)-5-hydroxy-N-isopentyl-1,4a,6a-trimethyl-2-oxo-2,3,4,4a,4b,5,6,6a,7,8,9,9a,9b,10-tetradecahydro-1H-indeno[5,4-f]quinoline-7-carboxamide, 36 (0.218 g, Off-white solid, 13.13% Yield). 1H-NMR (300 MHz, CDCl₃) δ: 5.28 (s, 1H), 4.89 (s, 1H), 4.41 (s, 1H), 3.32-3.24 (m, 2H), 3.11 (s, 3H), 2.63-2.57 (m, 2H), 2.15-1.86 (m, 5H), 1.85-1.77 (m, 4H), 1.59-1.46 (m, 2H), 1.43-1.35 (m, 4H), 1.33 (s, 3H), 1.25-1.21 (m, 3H), 1.16-1.08 (s, 3H), 0.93 (s, 6H); ESI-MS (m/z) Calcd for C₂₅H₄₀N₂O₃: 416.61 Found 417.3 [M+H]+; HPLC purity: 99.15%.

### Example 3: Synthesis of Compound 40

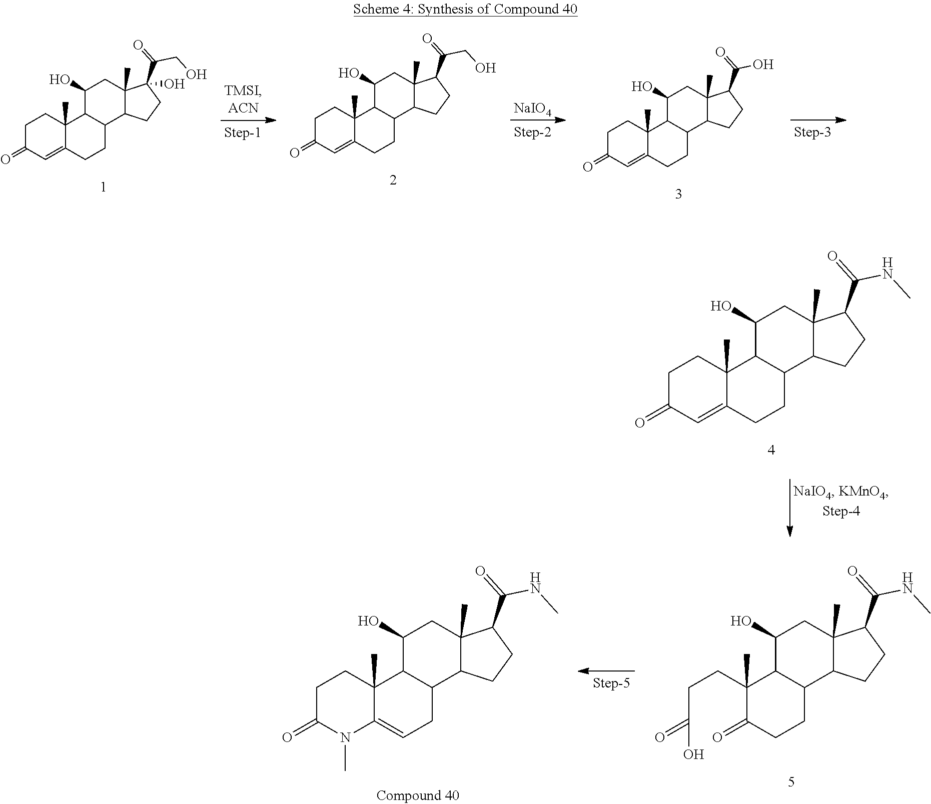

### 1. Synthesis of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one (2)

To a stirred solution of (10R,11S,13S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one (1, 30 g, 82.87 mmol, 1.0 eq) in acetonitrile (500 mL), was added TMSI (59.2 mL, 414.36 mmoL, 5 eq) and stirred at room temperature for 2 hrs. After completion of reaction, the reaction mixture was poured into saturated sodium bisulphite solution and removed the organics under reduced pressure. Then, obtained crude was washed with EtOAc and, separated organic layer was dried over sodium sulfate and concentrated to obtain (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one (2) as a yellow solid (25 g, 87.4%). ESI-MS: calculated m/z 346.21 [M]+, observed m/z 347.4 [M+H]+.

### 2. Synthesis of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (3)

To a stirred suspension of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one (2, 25 g, 72.25 mmol, 1.0 eq) in acetone:water (700 mL:800 mL), NaIO₄ (61.2 g, 289.01 mmoL, 4 eq) was added. The reaction was stirred at room temperature for 2 hrs. After completion of reaction, removed the organics under reduced pressure. Then, obtained aqueous was acidified using 2N HCl and extracted with dichloromethane. Separated organic layer was dried over sodium sulfate and concentrated. Crude was purified using 100-200 mesh silica gel and 2% methanol in dichloromethane as eluent to obtain (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (3) as a yellow solid (22.1 g, 91.7%). ESI-MS: calculated m/z 332.20 [M]+, observed m/z 331.2 [M+H]+.

### 3. Synthesis of (10R,11S,13S)-11-hydroxy-N,10,13-trimethyl-3-oxo-2,3,6,7,8,9,10,11,12,13,14,15,16,17-tetradecahydro-1H-cyclopenta[a]phenanthrene-17-carboxamide (4)

To a stirred solution of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (3.5 g, 15.11 mmol, 1.0 eq) in N,N-Dimethyl formamide (50 mL), DIPEA (13 mL, 75.55 mmol, 5.0 eq), EDC·HCl (4.3 g, 22.67 mmol, 1.5 eq) and HOBt (3 g, 22.67 mmol, 1.5 eq) were added. The reaction was stirred at room temperature for 15 minutes. Added methylamine hydrochloride (1.0 g, 15.11 mmol, 1.1 eq) and continued stirring for 18 hrs. After completion of reaction, poured reaction mixture into cold water and worked up with ethyl acetate (50 mL) and water. Organic layer was dried over Na₂SO₄ and concentrated under reduced pressure. Crude was purified using Silica gel and 2% MeOH/DCM as eluent to obtain (10R,11S,13S)-11-hydroxy-N,10,13-trimethyl-3-oxo-2,3,6,7,8,9,10,11,12,13,14,15,16,17-tetradecahydro-1H-cyclopenta[a]phenanthrene-17-carboxamide (4) as pale yellow solid (2.4 g, 46.15%). ESI-MS: calculated m/z 345.23 [M]+, observed m/z 346.2 [M+1]+.

### 4. Synthesis of 3-((3aS,5S,6R)-5-hydroxy-3a,6-dimethyl-3-(methylcarbamoyl)-7-oxododecahydro-1H-cyclopenta[a]naphthalen-6-yl)propanoic acid (5)

To a stirred suspension of (10R,11S,13S)-11-hydroxy-N,10,13-trimethyl-3-oxo-2,3,6,7,8,9,10,11,12,13,14,15,16,17-tetradecahydro-1H-cyclopenta[a]phenanthrene-17-carboxamide (4, 2.8 g, 2.91 mmol, 1.0 eq) in t-butanol (40 mL). Added a solution of potassium carbonate (2.02 g, 8.16 mmol, 1.8 eq) in water (20 mL) to the stirred reaction mixture.. Then, the reaction was heated at 50° C. for 30 minutes and added a solution of sodium metaperiodate (8.7 g, 40.81 mmol, 5.0 eq) and potassium permanganate (193 mg, 1.22, 0.15 eq). After completion of reaction, reaction mixture was concentrated under reduced pressure to remove organics. Then obtained aqueous layer was filtered off, washed with water (50×2 mL). The filtrate was acidified using 2N HCl and extracted using dichloromethane. Separated organic layer was dried over Na₂SO₄ and concentrated under reduced pressure to obtain 3-((3aS,5S,6R)-5-hydroxy-3a,6-dimethyl-3-(methylcarbamoyl)-7-oxododecahydro-1H-cyclopenta[a]naphthalen-6-yl)propanoic acid (5) as a white solid (1.4 g, 47.13%). ESI-MS: calculated m/z 365.22 [M]+, observed m/z 366.2 [M+H]+.

### 5. Synthesis of (4aR,5S,6aS)-5-hydroxy-N,1,4a,6a-tetramethyl-2-oxo-2,3,4,4a,4b,5,6,6a,7,8,9,9a,9b,10-tetradecahydro-1H-indeno[5,4-f] quinoline-7-carboxamide (Compound 40)

To a stirred solution of 3-((3aS,5S,6R)-5-hydroxy-3a,6-dimethyl-3-(methylcarbamoyl)-7-oxododecahydro-1H-cyclopenta[a]naphthalen-6-yl)propanoic acid (5, 1.4 g, 3.85 mmol, 1.0 eq) in N,N-Dimethylformamide (25 mL), methylamine hydrochloride (240 mg, 38.50 mmol, 10.0 eq) and triethylamine (7.7 mL, 57.75 mmoL, 15.0 eq) were added and heated in a sealed tube at 120° C. for 18 hrs. The reaction was refluxed at 120° C. for 16 hrs. After completion of reaction, the reaction mixture was cooled at rt, worked up using ice-water and ethyl acetate. Separated organic layer was dried over Na₂SO₄ and concentrated under reduced pressure then the solid precipitated in the reaction mixture was filtered off and washed with water (50×2 mL) then with 30 mL of Methanol. Thus obtained crude amount was purified by column chromatography using silica gel and 2% MeOH/DCM as eluent to obtain (4aR,5S,6aS)-5-hydroxy-N,1,4a,6a-tetramethyl-2-oxo-2,3,4,4a,4b,5,6,6a,7,8,9,9a,9b,10-tetradecahydro-1H-indeno[5,4-f]quinoline-7-carboxamide as pale yellow colour solid (360 mg, 26%). ESI-MS: calculated m/z 360.24 [M]+, observed m/z 361.4 [M+H]+. 1H NMR (300 MHz, DMSO): δ/ppm 7.67-7.66 (q, 0.26H), 7.35-7.33 (q, 0.73H), 4.85 (t, 1H), 4.21 (s, 2H), 2.98 (s, 3H), 2.57-2.56 (d, 3H), 2.07 (s, 3H), 1.92-1.23 (m, 9H), 1.22 (s, 3H), 1.01-1.21 (m, 5H), 0.82 (s, 3H).

### Example 4: Synthesis of Compound 50

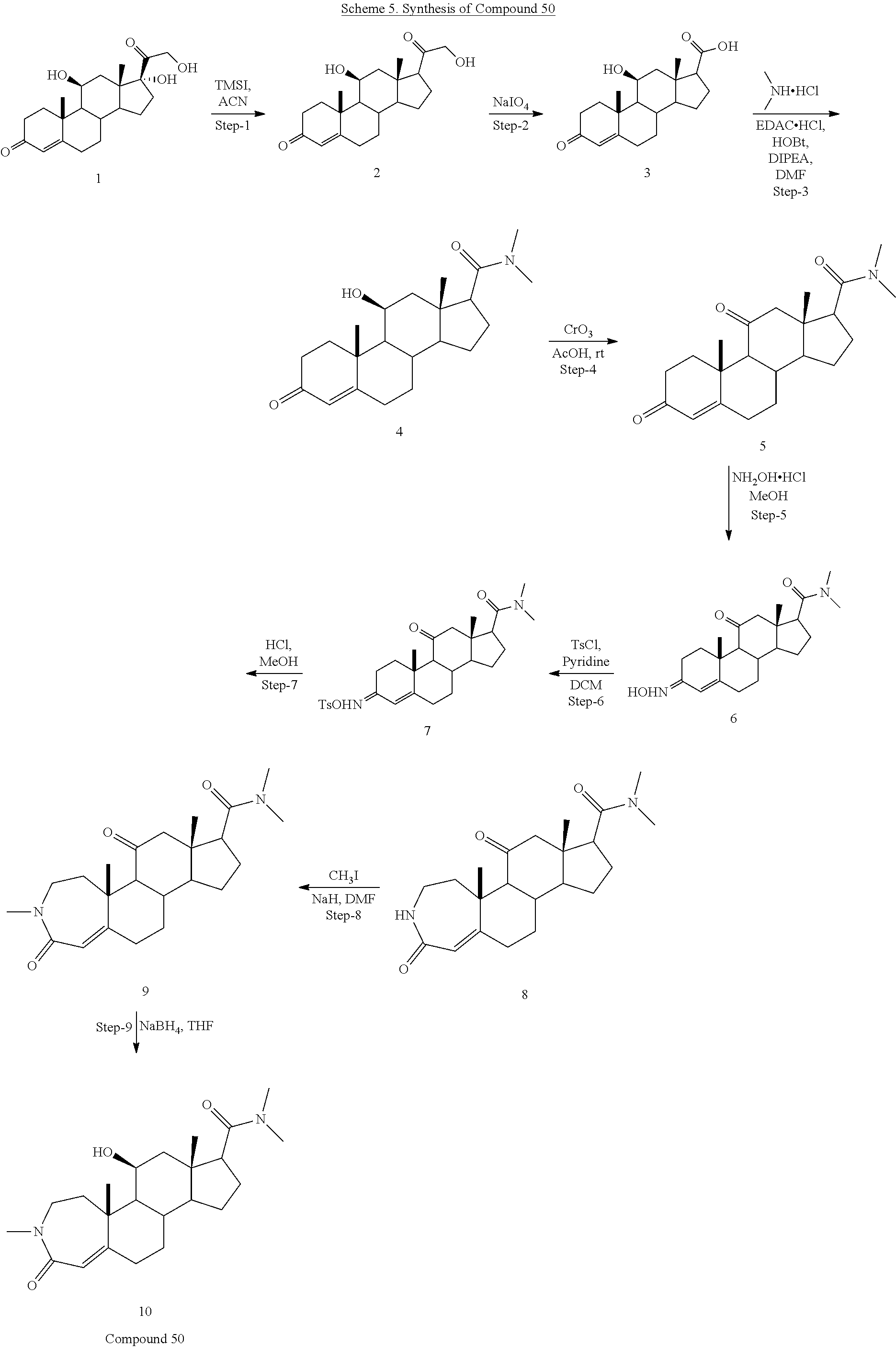

### 1. Synthesis of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one (2)

To a stirred solution of (10R,11S,13S,17R)-11,17-dihydroxy-17-(2-hydroxyacetyl)-10,13-dimethyl-1,2,6,7,8,9,10,11,12,13,14,15,16,17-tetradecahydro-3H-cyclopenta[a]phenanthren-3-one, 1 (30 g, 82.87 mmol, 1.0 eq) in acetonitrile (500 mL), was added TMSI (59.2 mL, 414.36 mmoL, 5 eq) and stirred at room temperature for 2 hrs. After completion of reaction, the reaction mixture was poured into saturated sodium bisulphite solution and removed the organics under reduced pressure. Then, obtained crude was washed with EtOAc and, separated organic layer was dried over sodium sulfate and concentrated to obtain (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one 2 (25 g, yellow solid, 87.4%). ESI-MS (m/z) Calcd for C₂₁H₃₀O₄: 346.21 Found 347.4 (M+H)+.

### 2. Synthesis of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid (3)

To a stirred suspension of (10R,11S,13S)-11-hydroxy-17-(2-hydroxyacetyl)-10,13-dimethyl-1,2,6,7,8,9,10,11,12,13,14,15,16,17-tetradecahydro-3H-cyclopenta[a]phenanthren-3-one, 2 (25 g, 72.25 mmol, 1.0 eq) in acetone:water (700 mL:800 mL), NaIO₄ (61.2 g, 289.01 mmoL, 4 eq) was added. The reaction was stirred at room temperature for 2 hrs. After completion of the reaction, solvent was removed under reduced pressure. Then, obtained aqueous was acidified using 2N HCl and extracted with dichloromethane. The organic layer was dried over anhyd. sodium sulfate, filtered and concentrated in vacuo to obtain the crude product which was subjected to column chromatography to obtain (silica gel: 100-200 mesh; 2% MeOH:DCM) to obtain (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid 3 (22.1 g, yellow solid, 91.7%). ESI-MS (m/z) Calcd for C₂₀H₂₈O₄: 332.20 Found 333.2 (M+H)+.

### 3. Synthesis of (10R,11S,13S)-11-hydroxy-N,N,10,13-tetramethyl-3-oxo-2,3,6,7,8,9,10,11,12,13,14,15,16,17-tetradecahydro-1H-cyclopenta[a]phenanthrene-17-carboxamide (4)

To a stirred solution of (10R,11S,13S)-11-hydroxy-10,13-dimethyl-3-oxo-2,3,6,7,8,9,10,11,12,13,14,15,16,17-tetradecahydro-1H-cyclopenta[a]phenanthrene-17-carboxylic acid, 3 (10.00 g, 30.12 mmol, 1.0 eq) dissolved in DMF (30 mL) was added DIPEA (17.3 mL, 100.58 mmol, 5.0 eq). This was followed by addition of EDC·HCl (8.6 g, 45.0 mmol, 1.5 eq) and HOBt (6 g, 45.0 mmol, 1.5 eq) at 0° C., followed by addition of 3-methylbutan-1-amine, (2.5 g, 30.65 mmol, 1.0 eq). The reaction mixture was slowly warmed to room temperature and stirred for 16 hours. After completion of the reaction, water was added to the reaction mixture to obtain precipitate which was filtered, dried and subjected to column chromatography to obtain (silica gel: 100-200 mesh; 1% MeOH:DCM) to obtain (10R,11S,13S)-11-hydroxy-N,N,10,13-tetramethyl-3-oxo-2,3,6,7,8,9,10,11,12,13,14,15,16,17-tetradecahydro-1H-cyclopenta[a]phenanthrene-17-carboxamide, 4 (7.74 g, 71.60% Yield). ESI-MS (m/z) Calcd for C₂₂H₃₃NO₃: 359.25 Found 360.2 (M+H)+.

### 4. Synthesis of (10R,13S)—N,N,10,13-tetramethyl-3,11-dioxo-2,3,6,7,8,9,10,11,12,13,14,15,16,17-tetradecahydro-1H-cyclopenta[a]phenanthrene-17-carboxamide (5)

To a stirred solution of (10R,11S,13S)-11-hydroxy-N,N,10,13-tetramethyl-3-oxo-2,3,6,7,8,9,10,11,12,13,14,15,16,17-tetradecahydro-1H-cyclopenta[a]phenanthrene-17-carboxamide, 4 (7.0 g, 19.49 mmol, 1.0 eq) in acetic acid (60 mL) was added aqueous solution (5 mL) of CrO₃ (2.3 g, 23.0 mmol, 1.1 eq) drop-wise at room temperature and nitrogen atmosphere. The reaction mixture was stirred for 1 hour followed by addition of MeOH (100 mL). The reaction mixture was stirred for another 30 minutes and on completion of the reaction; the reaction mixture was poured into saturated solution of NaHCO₃ and extracted using ethyl acetate. The organic layer was dried over anhyd. sodium sulfate, filtered and concentrated in vacuo to obtain (10R,13S)—N,N,10,13-tetramethyl-3,11-dioxo-2,3,6,7,8,9,10,11,12,13,14,15,16,17-tetradecahydro-1H-cyclopenta[a]phenanthrene-17-carboxamide, 5 (6.7 g, 96.26% Yield). ESI-MS (m/z) Calcd for C₂₂H₃₁NO₃: 357.23 Found 358.2 (M+H)+.

### 5. Synthesis of (10R,13S,Z)-3-(hydroxy-14-azaneylidene)-N,N,10,13-tetramethyl-11-oxo-2,3,6,7,8,9,10,11,12,13,14,15,16,17-tetradecahydro-1H-cyclopenta[a]phenanthrene-17-carboxamide (6)

To a stirred solution of (10R,13S)—N,N,10,13-tetramethyl-3,11-dioxo-2,3,6,7,8,9,10,11,12,13,14,15,16,17-tetradecahydro-1H-cyclopenta[a]phenanthrene-17-carboxamide, 5 (6.7 g, 20.0 mmol, 1.0 eq) in methanol (180 mL) was added pyridine (4.75 mL, 60.0 mmol, 3.0 eq) and hydroxylammonium chloride (1.6 g, 23.0 mmol, 1.2 eq) at 0° C. Then reaction mixture was slowly warmed to room temperature and then heated at 50° C. for 2 hours. After completion of reaction, the solvent was evaporated in-vacuo. Then, water was added to the reaction mixture and extracted with ethyl acetate. The organic layer was dried over anhyd. sodium sulfate, filtered and concentrated in vacuo to obtain (10R,13S,Z)-3-(hydroxy-14-azaneylidene)-N,N,10,13-tetramethyl-11-oxo-2,3,6,7,8,9,10,11,12,13,14,15,16,17-tetradecahydro-1H-cyclopenta[a]phenanthrene-17-carboxamide, 6 (6.95 g, 99.5% Yield). ESI-MS (m/z) Calcd for C₂₂H₃₃N₂O₃: 373.25.

### 6. Synthesis of ((10R,13S,Z)-17-(dimethylcarbamoyl)-10,13-dimethyl-11-oxo-1,2,6,7,8,9,10,11,12,13,14,15,16,17-tetradecahydro-3H-cyclopenta[a]phenanthren-3-ylidene)-14-azaneyl 4-methylbenzenesulfonate (7)

To a stirred solution of (10R,13S,Z)-3-(hydroxy-14-azaneylidene)-N,N,10,13-tetramethyl-11-oxo-2,3,6,7,8,9,10,11,12,13,14,15,16,17-tetradecahydro-1H-cyclopenta[a]phenanthrene-17-carboxamide, 6 (7.0 g, 20.0 mmol, 1.0 eq) in dichloromethane (180 mL) was added pyridine (39 mL, 475.0 mmol, 25.0 eq) and tosylchloride (4.3 g, 220.0 mmol, 1.2 eq). The reaction mixture was then stirred at room temperature for overnight. After completion of the reaction, water was added to reaction mixture and the compound was extracted using dichloromethane. The organic layer was dried over anhyd. sodium sulfate, filtered and concentrated in vacuo to obtain the crude product which was subjected to column chromatography to obtain (silica gel: 100-200 mesh; 25% ethyl acetate:hexane) to obtain ((10R,13S,Z)-17-(dimethylcarbamoyl)-10,13-dimethyl-11-oxo-1,2,6,7,8,9,10,11,12,13,14,15,16,17-tetradecahydro-3H-cyclopenta[a]phenanthren-3-ylidene)-14-azaneyl 4-methylbenzenesulfonate, 7 (9 g, 91.09% Yield). ESI-MS (m/z) Calcd for C₂₉H₃₉N₂O₅S: 527.26 Found 527.2 (M+H)+.

### 7. Synthesis of (5aR,7aS)—N,N,5a,7a-tetramethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide (8)

To a stirred solution of ((10R,13S,Z)-17-(dimethylcarbamoyl)-10,13-dimethyl-11-oxo-1,2,6,7,8,9,10,11,12,13,14,15,16,17-tetradecahydro-3H-cyclopenta[a]phenanthren-3-ylidene)-14-azaneyl 4-methylbenzenesulfonate, 7 (9.0 g, 24.19 mmol, 1.0 eq) in methanol (300 mL) was added concentrated HCl (90 mL) drop-wise. The reaction mixture was then stirred at 50° C. for 12 hours. After completion of the reaction, the solvent was evaporated in-vacuo. Then, cold saturated solution of NaHCO₃ was added to reaction mixture and extracted with dichloromethane. The organic layer was dried over anhyd. sodium sulfate, filtered and concentrated in vacuo to obtain the crude product which was subjected to column chromatography to obtain (silica gel: 100-200 mesh; 1% Methanol:DCM) to obtain (5aR,7aS)—N,N,5a,7a-tetramethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide, 8 (1 g, 15.74% Yield). ESI-MS (m/z) Calcd for C₂₂H₃₂N₂O₃: 372.24 Found 373.2 (M+H)+.

### 8. Synthesis of (5aR,7aS)—N,N,3,5a,7a-pentamethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide (9)

To a stirred solution of (5aR,7aS)—N,N,5a,7a-tetramethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide, 8 (0.95 g, 2.55 mmol, 1.0 eq) in DMF (5 mL) was added sodium hydride (0.176 g, 7.33 mmol, 2.8 eq) at 0° C. Then methyl iodide (0.19 mL, 3.06 mmol, 1.2 eq) was added to the reaction mixture drop-wise. After completion of the reaction, reaction mixture was added to ice-cold water and compound was extracted using ethyl acetate. The organic layer was dried over anhyd. sodium sulfate, filtered and concentrated in vacuo to obtain the crude product which was subjected to column chromatography to obtain (silica gel: 100-200 mesh; 1% Methanol:DCM) (5aR,7aS)—N,N,3,5a,7a-pentamethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide, 9 (0.650 g, 65.98% Yield). ESI-MS (m/z) Calcd for C₂₃H₃₄N₂O₃: 386.26 Found 387.3 (M+H)+.

### 9. Synthesis of (5aR,6S,7aS)-6-hydroxy-N,N,3,5a,7a-pentamethyl-2-oxo-2,3,4,5,5a,5b,6,7,7a,8,9,10,10a,10b,11,12-hexadecahydrocyclopenta[5,6]naphtho[1,2-d]azepine-8-carboxamide (Compound 50)

To a stirred solution of (5aR,7aS)—N,N,3,5a,7a-pentamethyl-2,6-dioxo-2,3,4,5,5a,5b,6,7,7a,8,9,10,10a,10b,11,12-hexadecahydrocyclopenta[5,6]naphtho[1,2-d]azepine-8-carboxamide, 9 (0.650 g, 1.68 mmol, 1.0 eq) in methanol (5 mL) and THF (5 mL) was added sodium borohydride (0.320 g, 8.4 mmol, 5.0 eq) at 0° C. Then the reaction mixture was stirred at 80° C. for 2 hours. After completion of the reaction, the solvent was evaporated in-vacuo. Then, ice-cold water was added to the reaction mixture and extracted with dichloromethane. The organic layer was dried over anhyd. sodium sulfate, filtered and concentrated in vacuo to obtain the crude product which was subjected to column chromatography to obtain (silica gel: 100-200 mesh; 2.5% Methanol:DCM) to obtain (5aR,6S,7aS)-6-hydroxy-N,N,3,5a,7a-pentamethyl-2-oxo-2,3,4,5,5a,5b,6,7,7a,8,9,10,10a,10b,11,12-hexadecahydrocyclopenta[5,6]naphtho[1,2-d]azepine-8-carboxamide, 10 (0.430 g, white solid, 65.84% Yield). 1H-NMR (300 MHz, DMSO-d6) δ: 5.50 (s, 1H), 4.23 (s, 1H), 4.08 (brs, 1H), 3.26-3.188 (m, 1H), 2.98 (s, 3H), 2.88 (s, 3H), 2.80 (s, 3H), 2.75-2.69 (m, 2H), 2.13-2.07 (m, 2H), 2.02-2.00 (m, 2H), 1.95-1.88 (m, 1H), 1.86-1.81 (m, 4H), 1.60-1.52 (m, 2H), 1.48-1.45 (m, 1H), 1.25 (s, 4H), 1.14-1.10 (m, 2H), 0.91 (s, 3H); ESI-MS (m/z) Calcd for C₂₃H₃₆N₂O₃: 388.27 Found 389.3 [M+H]+; HPLC purity: 96.15%.

### Example 5: Characterization of Compounds

Additional compounds were prepared. Their characterization data is presented in Table 2.

TABLE 2
Characterization of prepared compounds.

| Compound | LCMS/1H NMR |
|---|---|
| (4aR,5S,6aS,7S)-5-hydroxy-7-((S)-1- | ¹H-NMR (300 MHz, DMSO-d₆) δ: 9.20 |
| hydroxyethyl)-4a,6a-dimethyl- | (s, 1H), 4.68 (s, 1H), 4.10 (s, 2H), 4.06 (d, J = |
| 1,3,4,4a,4b,5,6,6a,7,8,9,9a,9b,10- | 5.4 Hz, 1H), 3.47-3.39 (m, 1H), 2.38-2.23 |
| tetradecahydro-2H-indeno[5,4-f]quinolin-2- | (s, 3H), 2.18-2.07 (m, 1H), 1.99-1.85 |
| one | (m, 2H), 1.65-1.50 (m, 3H), 1.44-1.38 |
| | (m, 2H), 1.21 (s, 3H), 1.16-1.06 (m, 3H), |
| | 0.99-0.97 (m, 5H), 0.87 (s, 3H). ESI-MS |
| | (m/z) Calcd for C₂₀H₃₁NO₃: 333.2. |
| | Found 334.4 [M + 1]⁺. |
| (4aR,5S,6aS)-5-hydroxy-1,4a,6a-trimethyl- | ¹H-NMR (300 MHz, DMSO-d₆) δ: 4.86 (bs, |
| 7-(2-methyl-1,3-dioxolan-2-yl)- | 1H), 4.16 (bs, 2H), 3.90-3.77 (m, 4H), |
| 1,3,4,4a,4b,5,6,6a,7,8,9,9a,9b,10- | 2.98 (s, 3H), 2.40-2.32 (m, 2H), 2.30-1.95 |
| tetradecahydro-2H-indeno[5,4-f]quinolin-2- | (m, 1H), 1.90-1.55 (m, 6H), 1.54-1.39 |
| one | (m, 2H), 1.37-1.06 (m, 10H), 0.93 (s, 4H). |
| | ESI-MS (m/z) Calcd for C₂₃H₃₅NO₄: |
| | 389.2. Found 390.1 [M + 1]⁺. |
| (4aR,5S,6aS,7S)-7-acetyl-5-hydroxy- | ¹H-NMR (300 MHz, MeOD) δ: 5.10 (s, 1H), |
| 1,4a,6a-trimethyl- | 4.41 (s, 1H), 3.12 (s, 3H), 2.45-2.40 |
| 1,3,4,4a,4b,5,6,6a,7,8,9,9a,9b,10- | (t, 1H), 2.33-2.32 (m, 1H), 2.28-2.23 |
| tetradecahydro-2H-indeno[5,4-f]quinolin-2- | (m, 2H), 2.20-1.19 (m, 1H), 2.14 (s, 3H), |
| one | 2.09-2.01 (m, 1H), 1.97-1.90 (m, 2H), |
| | 1.72-1.68 (m, 4H), 1.32-1.29 (m, 20H), |
| | 0.89-0.82 (s, 8H). ESI-MS (m/z) Calcd for |
| | C₂₁H₃₁NO₃: 345.2, Found 346.0 [M + 1]⁺. |
| (4aR,5S,6aS)-7-acetyl-5-hydroxy-1,4a,5,6a- | ¹H-NMR (300 MHz, CDCl₃) δ: 5.03-5.01 |
| tetramethyl- | (t, 1H), 3.12 (s, 3H), 2.74-2.70 (m, 3H), |
| 1,3,4,4a,4b,5,6,6a,7,8,9,9a,9b,10- | 2.67-2.65 (m, 2H), 2.47-2.35 (m, 2H), |
| tetradecahydro-2H-indeno[5,4-f]quinolin-2- | 2.12 (s, 3H), 2.01-1.93 (m, 2H), 1.90-1.87 |
| one | (m, 4H), 1.57-1.56 (m, 1H), 1.40-1.37 |
| | (m, 3H), 1.25-1.23 (m, 5H), 0.87-0.85 |
| | (m, 1H), 0.63 (s, 3H). ESI-MS (m/z) |
| | Calcd for C₂₂H₃₃NO₃: 359.1. Found 359.9 |
| | [M + 1]⁺. |
| (4aR,5S,6aS,7S)-N-cyclobutyl-5-hydroxy- | ¹H-NMR (300 MHz, DMSO-d₆) δ: 7.67 (d, |
| 1,4a,6a-trimethyl-2-oxo- | J = 7.5 Hz, 1H), 4.91 (s, 1H), 4.28 (s, 4H), |
| 2,3,4,4a,4b,5,6,6a,7,8,9,9a,9b,10- | 3.03 (s, 3H), 2.48-2.27 (m, 2H), 2.19-2.16 |
| tetradecahydro-1H-indeno[5,4-f]quinoline- | (m, 3H), 2.07-1.80 (m, 7H), 1.68-1.40 |
| 7-carboxamide | (m, 5H), 1.30 (s, 3H), 1.29-1.10 (m, 2H), |
| | 1.09-1.00 (m, 2H), 0.80 (s, 3H). ESI-MS |
| | (m/z) Calcd for C₂₄H₃₆N₂O₃: 400.5. |
| | Found 401.3 [M + 1]⁺. HPLC purity: 95.40%. |
| N-((4aR,5S,6aS,7S)-5-hydroxy-1,4a,6a- | ¹H-NMR (300 MHz, CDCl₃) δ: 5.65 (s, 1H), |
| trimethyl-2-oxo- | 4.40-4.39 (m, 1H), 4.00 (d, J = 9.6 Hz, |
| 2,3,4,4a,4b,5,6,6a,7,8,9,9a,9b,10- | 1H), 3.47 (d, J = 5.4 Hz, 1H), 3.18-3.15 |
| tetradecahydro-1H-indeno[5,4-f]quinolin-7- | (m, 1H), 3.02-2.95 (q, J = 7.5 Hz, 2H),), |
| yl)ethanesulfonamide | 2.45-2.10 (m, 6H), 2.19-2.00 (m, 4H), 1.50 |
| | (s, 3H), 1.34 (t, J = 7.5 Hz, 3H), 1.31-1.23 |
| | (m, 7H) 0.97 (s, 3H), ESI-MS (m/z) Calcd for |
| | C₂₁H₃₃NO₄S: 395.2 Found 396.1 [M + 1]⁺. |
| | HPLC purity: 98.16%. |
| (4aR,5S,6aS,7S)-7-amino-5-hydroxy- | ¹H NMR (300 MHz, CDCl₃): δ/ppm 4.89 |
| 1,4a,6a-trimethyl- | (s, 1 H), 4.89-4.86 (m, 1H), 3.09 (s, 3 H), |
| 1,3,4,4a,4b,5,6,6a,7,8,9,9a,9b,10- | 2.78 (d, J = 4.8 Hz 1H), 2.64-2.55 (m, 4H), |
| tetradecahydro-2H-indeno[5,4-f]quinolin-2- | 2.47-2.38 (m, 2H), 2.16-1.86 (m, 6H), |
| one | 1.26-1.23 (m, 8H), 0.91 (s, 3H), 0.89-0.86 |
| | (m, 2H), ESI-MS: calculated C₁₉H₃₀N₂O₂: |
| | 318.2 m/z observed m/z 319.1 [M + H]⁺. |
| N-((4aR,5S,6aS,7S)-5-hydroxy-1,4a,6a- | ¹H-NMR (300 MHz, DMSO-d₆) δ: 7.71 (d, |
| trimethyl-2-oxo- | J = 8.7, 1H), 4.86 (s, 1H), 4.86 (s, 1H), 3.67 |
| 2,3,4,4a,4b,5,6,6a,7,8,9,9a,9b,10- | (q, J = 9.3 Hz, 1H), 3.3 (s, 1H), 2.98 (s, 3H), |
| tetradecahydro-1H-indeno[5,4-f]quinolin-7- | 2.51-2.28 (m, 5H), 1.87-1.74(s, 4H), 1.64-1.61 |
| yl)cyclopropanecarboxamide | (m, 2H), 1.48-1.44 (m, 2H), 1.27-1.24 |
| | (m, 3H), 1.21 (s, 3H), 1.13 (m, 1H), 0.91 |
| | (s, 3H), 0.63-0.59 (m, 4H). ESI-MS (m/z) |
| | Calcd for C₂₃H₃₄N₂O₃: 386.54 Found 387.2 |
| | (M + H)⁺, HPLC purity: 98.5%. |
| N-((4aR,5S,6aS,7S)-5-hydroxy-1,4a,6a- | ¹H-NMR (300 MHz, CDCl₃) δ: 5.17 (m, 1H), |
| trimethyl-2-oxo- | 4.86 (s, 1H), 4.86 (s, 1H), 4.87 (bs, 1H), |
| 2,3,4,4a,4b,5,6,6a,7,8,9,9a,9b,10- | 4.36 (bs, 1H), 3.85-3.83 (m, 1H), 3.71-3.66 |
| tetradecahydro-1H-indeno[5,4-f]quinolin-7- | (m, 4H), 3.16-3.13 (m, 4H), 3.08(s, 3H), |
| yl)cyclobutanecarboxamide | 2.91-3.02 (m, 1H), 2.64-2.48 (m, 2H), |
| | 2.27-1.34 (m, 6H), 1.21 (s, 3H), 1.13 (m, 1H), |
| | 0.91 (s, 3H), 0.85-0.82 (m, 4H). ESI-MS |
| | (m/z) Calcd for C₂₄H₃₆N₂O₃: 400.27 |
| | Found 401.1(M + H)⁺, HPLC purity: 95.74%. |
| (4aR,5S,6aS)-N-(cyclopropylmethyl)-5- | ¹H-NMR (300 MHz, DMSO-d₆) δ: 7.29 (bs, 1H), |
| hydroxy-1,4a,6a-trimethyl-2-oxo- | 4.70 (bs, 1H), 4.07 (bs, 2H), 2.82-2.73 |
| 2,3,4,4a,4b,5,6,6a,7,8,9,9a,9b,10- | (m, 6H), 2.27 (bs, 3H), 2.21-2.11 (m, 3H), |
| tetradecahydro-1H-indeno[5,4-f]quinoline- | 1.92-1.81 (m, 3H), 1.65-1.25 (m, 8H), |
| 7-carboxamide | 1.17-0.64 (m, 7H), 0.23-0.19 (m, 2H). |
| | ESI-MS (m/z) Calcd for C₂₄H₃₆N₂O₃: 400.5. |
| | Found 401.3 [M + 1]⁺. HPLC purity: 97.20%. |
| (4aR,5S,6aS)-5-hydroxy-N-isopentyl- | ¹H-NMR (300 MHz, DMSO-d₆) δ: 7.35 (bs, 1H), |
| 1,4a,6a-trimethyl-2-oxo- | 4.85 (bs, 1H), 4.25-4.20 (m, 2H), 3.13-3.09 |
| 2,3,4,4a,4b,5,6,6a,7,8,9,9a,9b,10- | (m, 2H), 2.98 (s, 3H), 2.89 (s, 1H), |
| tetradecahydro-1H- | 2.42-2.34 (m, 2H), 2.28-2.27 (m, 2H), |
| indeno[5,4-f]quinoline-7-carboxamide | 2.03-1.42 (m, 15H), 1.38-1.20 (m, 5H), |
| | 1.00-0.85 (m, 6H). ESI-MS (m/z) |
| | Calcd for C₂₅H₄₀N₂O₃: 416.6. Found 417.3 |
| | [M + 1]⁺. HPLC purity: 97.41%. |
| (4aR,5S,6aS,7S)-5-hydroxy-1,4a,6a- | ¹H-NMR (300 MHz, CDCl₃) δ: 4.91 (s, 1H), |
| trimethyl-2-oxo- | 4.45 (s, 3H), 3.12 (s, 3H), 2.72-2.55 (m, 3H), |
| 2,3,4,4a,4b,5,6,6a,7,8,9,9a,9b,10- | 2.52-2.35 (m, 2H), 2.12-1.34 (s, 3H), |
| tetradecahydro-1H-indeno[5,4-f]quinoline- | 2.01-1.93 (m, 2H), 1.90-1.87 (m, 4H), |
| 7-carboxylic acid | 1.57-1.56 (m, 1H), 1.40-1.37 (m, 3H), |
| | 1.12 (s, 3H), 0.87-0.65 (m, 3H). ESI-MS |
| | (m/z) Calcd for C₂₀H₂₉NO₄: 347.2. Found |
| | 348.2 [M + 1]⁺. |
| (4aR,5S,6aS)-5-hydroxy-N,1,4a,6a- | ¹H-NMR (300 MHz, CDCl₃) δ: 5.38 (bs, 1H), |
| tetramethyl-2-oxo- | 4.93-4.90 (m, 1H), 4.43 (s, 1H), 3.15 |
| 2,3,4,4a,4b,5,6,6a,7,8,9,9a,9b,10- | (s, 3H), 2.65-2.63 (m, 3H), 2.47-2.39 (m, 2H), |
| tetradecahydro-1H- | 2.20-1.97 (m, 6H), 1.76 (s, 3H), 1.53-1.48 |
| indeno[5,4-f]quinoline-7-carboxamide | (m, 2H), 1.45-1.38 (m, 2H), 1.18-1.06 |
| | (m, 5H), 1.00 (s, 3H). ESI-MS (m/z) |
| | Calcd for C₂₁H₃₂N₂O₃: 360.2. |
| (4aR,5S,6aS,7S)-5-hydroxy-7-methoxy- | ¹H-NMR (300 MHz, DMSO-d₆) δ: 4.24(s, 1H), |
| 1,4a,6a-trimethylhexadecahydro-2H- | 3.59 (bs, 1H), 3.55 (s, 4H), 3.53 (s, 1H), |
| indeno[5,4-f]quinolin-2-one | 3.26 (s, 3H), 3.12 (t, 1H), 2.87-2.85 |
| | (m, 3H), 2.42-2.39 (m, 1H), 1.97-1.94 |
| | (m, 4H), 1.90-1.84 (m, 1H), 1.47-1.40 |
| | (m, 5H), 1.28-1.21 (m, 3H), 1.04-0.93 |
| | (m, 3H), 0.75-7.71 (m, 2H). ESI-MS (m/z) |
| | Calcd for C₂₀H₃₃NO₃: 335.4. Found 336.0 [M]⁺. |
| methyl (5aR,6S,7aS)-6-hydroxy-5a,7a- | ¹H-NMR (300 MHz, CDCl₃) δ: 6.12 (brs, 1H), |
| dimethyl-2-oxo- | 5.63 (s, 1H), 4.26(bs, 1H), 3.65 (s, 3H), |
| 2,3,4,5,5a,5b,6,7,7a,8,9,10,10a,10b,11,12- | 3.39-3.26 (m, 1H), 3.25-3.16 (m, 1H), |
| hexadecahydrocyclopenta[5,6]naphtho[1,2- | 2.58-2.49 (m, 1H), 2.30-2.13 (m, 5H), |
| d]azepine-8-carboxylate | 2.09-.1.79 (m, 6H), 1.35 (s, 3H), |
| | 1.47-1.1.01 (m, 5H), 0.97 (s, 3H). |
| | ESI-MS (m/z) Calcd for C₂₁H₃₁NO₄: 361.4. |
| | Found 362.0 [M + 1]⁺. HPLC purity: 95.08%. |
| (5aR,6S,7aS)-6-hydroxy-5a,7a-dimethyl-2- | ¹H-NMR (300 MHz, DMSOD₆) δ: 11.89 |
| oxo- | (bs, 1H exchanges with D2O), 7.70 (brs, |
| 2,3,4,5,5a,5b,6,7,7a,8,9,10,10a,10b,11,12- | 1H), 5.39 (s, 1H), 4.23 (s, 1H), 4.08 (s, 1H), |
| hexadecahydrocyclopenta[5,6]naphtho[1,2- | 3.03-2.72 (m, 3H), 2.72 (s, 1H), 2.63-2.49 |
| d]azepine-8-carboxylic acid | (m, 2H), 2.47-2.21 (m, 8H), 1.33 (s, 3H), |
| | 1.31-0.99 (m, 4H), 0.97 (s, 3H). ESI-MS |
| | (m/z) Calcd for C₂₀H₂₉NO₄: 347.4. Found |
| | 348.0 [M + 1]⁺. HPLC purity: 92.23%. |
| (5aR,6S,7aS)-N-cyclobutyl-6-hydroxy- | ¹H-NMR (300 MHz, CDCl₃) δ: 5.94 (brs, 1H), |
| 5a,7a-dimethyl-2-oxo- | 5.60 (s, 1H), 5.37-5.34 (d, 1H), 4.35-4.30 |
| 2,3,4,5,5a,5b,6,7,7a,8,9,10,10a,10b,11,12- | (m, 1H), 4.24 (brs, 1H), 3.27-3.22 (m, 1H), |
| hexadecahydrocyclopenta[5,6]naphtho[1,2- | 3.15-3.07 (m, 1H), 2.52-2.46 (m, 1H), |
| d]azepine-8-carboxamide | 2.33-2.29 (m, 2H), 2.26-1.70 (m, 14H), |
| | 1.69-1.64 (m, 6H), 1.47-1.42 (m, 2H), |
| | 1.33 (s, 3H), 1.20-1.02 (m, 4H), 0.82 |
| | (s, 3H). ESI-MS (m/z) Calcd for C₂₄H₃₆N₂O₃: |
| | 400.2. Found 401.0 [M + 1]⁺. HPLC purity: |
| | 96.47%. |
| (5aR,6S,7aS,8S)-6-hydroxy-N,N,5a,7a- | ¹H-NMR (300 MHz, CDCl₃) δ: 6.10 (brs, 1H), |
| tetramethyl-2-oxo- | 5.64 (s, 1H), 5.27 (s, 1H), 4.25 (s, 1H), |
| 2,3,4,5,5a,5b,6,7,7a,8,9,10,10a,10b,11,12- | 3.35-3.26 (m, 1H), 3.18-3.11 (m, 1H), 3.01 |
| hexadecahydrocyclopenta[5,6]naphtho[1,2- | (s, 3H), 2.93 (s, 3H), 2.64-2.50 (m, 2H), |
| d]azepine-8-carboxamide | 2.25-1.90 (m, 6H), 1.36 (s, 4H), 1.22 (s, 3H), |
| | 1.11-0.98 (m, 4H), 0.88 (s, 3H). ESI-MS |
| | (m/z) Calcd for C₂₂H₃₄N₂O₃: 374.2. |
| | Found 375.0 [M + 1]⁺. HPLC purity: 92.33%. |
| N-((4aR,5S,6aS,7S)-5-hydroxy-1,4a,6a- | ¹H-NMR (300 MHz, DMSO-d₆) δ: 5.50 (s, 1H), |
| trimethyl-2-oxo- | 4.23 (s, 1H), 4.08 (brs, 1H), 3.26-3.188 |
| 2,3,4,4a,4b,5,6,6a,7,8,9,9a,9b,10- | (m, 1H), 2.98 (s, 3H), 2.88 (s, 3H), 2.80 |
| tetradecahydro-1H- | (s, 3H), 2.75-2.69 (m, 2H), 2.13-2.07 (m, 2H), |
| indeno[5,4-f]quinolin-7- | 2.02-2.00 (m, 2H), 1.95-1.88 (m, 1H), 1.86-1.81 |
| yl)cyclopropanecarboxamide | (m, 4H), 1.60-1.52 (m, 2H), 1.48-1.45 (m, 1H), |
| | 1.25 (s, 4H), 1.14-1.10 (m, 2H), 0.91 (s, 3H); |
| | ESI-MS (m/z) Calcd for C₂₃H₃₆N₂O₃: 388.27 |
| | Found 389.3 [M + H]⁺; HPLC purity: 96.15%. |
| methyl (5aR,6S,7aS,8S)-6-hydroxy-3,5a,7a- | ¹H-NMR (300 MHz, CDCl₃) δ: 5.70 (s, 1H), |
| trimethyl-2-oxo- | 4.24 (s, 1H), 3.64 (s, 3H), 3.51-3.43 (m, 1H), |
| 2,3,4,5,5a,5b,6,7,7a,8,9,10,10a,10b,11,12- | 3.20-3.12 (m, 1H), 3.02 (s, 3H), 2.56-2.47 |
| hexadecahydrocyclopenta[5,6]naphtho[1,2- | (m, 1H), 2.28-1.72 (m, 9H), 1.67 (s, 2H), |
| d]azepine-8-carboxylate | 1.31 (s, 3H), 1.19-0.99 (m, 4H), 0.88 |
| | (s, 3H), 0.74-0.72 (m, 1H). ESI-MS (m/z) |
| | Calcd for C₂₂H₃₃NO₄: 375.2. Found 376.0 |
| | [M + 1]⁺. HPLC purity: 92.64%. |
| (5aR,6S,7aS,8S)-N-cyclopropyl-6-hydroxy- | ¹H-NMR (300 MHz, CDCl₃) δ: 5.68 (s, 1H), |
| 3,5a,7a-trimethyl-2-oxo- | 5.55 (s, 1H), 4.23 (s, 1H), 3.51-3.43 (m, 1H), |
| 2,3,4,5,5a,5b,6,7,7a,8,9,10,10a,10b,11,12- | 3.18-3.14 (m, 1H), 3.01 (s, 3H), 2.66 |
| hexadecahydrocyclopenta[5,6]naphtho[1,2- | (s, 1H), 2.54-2.46 (m, 1H), 2.22-2.12 (m, 1H), |
| d]azepine-8-carboxamide | 2.08-1.70 (m, 12H), 1.30 (s, 3H), 1.22 |
| | (s, 3H), 0.90 (s, 3H), 0.74-0.72 (m, 2H), |
| | 0.44 (s, 2H). ESI-MS (m/z) Calcd for |
| | C₂₄H₃₆N₂O₃: 400.2. Found 401.1 [M + 1]⁺. |
| | HPLC purity: 97.72%. |
| N-((5aR,6S,7aS,8S)-6-hydroxy-3,5a,7a- | ¹H-NMR (300 MHz, DMSO-d₆) δ: 7.68 (d, |
| trimethyl-2-oxo- | J = 8.1 Hz, 1H), 5.49 (s, 1H), 4.13 (s, 1H), |
| 2,3,4,5,5a,5b,6,7,7a,8,9,10,10a,10b,11,12- | 4.03-4.01 (m, 1H), 3.64-3.61 (m, 1H), 3.26-3.23 |
| hexadecahydrocyclopenta[5,6]naphtho[1,2- | (m, 2H), 2.89 (s, 3H), 2.27 (s, 2H), |
| d]azepin-8-yl)cyclopropanecarboxamide | 1.98-1.45 (m, 12H), 1.25 (s, 3H), 1.19-1.13 |
| | (m, 2H), 0.89 (s, 3H), 0.62-0.58 (m, 4H). |
| | ESI-MS (m/z) Calcd for C₂₄H₃₆N₂O₃: 400.2. |
| | Found 401.3 [M + 1]⁺. HPLC purity: 95.96%. |
| N-((5aR,6S,7aS,8S)-6-hydroxy-3,5a,7a- | ¹H-NMR (300 MHz, DMSO-d₆) δ: 7.22 (d, |
| trimethyl-2-oxo- | J = 8.7 Hz, 1H), 5.49 (s, 1H), 4.14 (s, 1H), |
| 2,3,4,5,5a,5b,6,7,7a,8,9,10,10a,10b,11,12- | 4.03 (s, 1H), 3.61-3.58 (m, 1H), 3.38-3.23 |
| hexadecahydrocyclopenta[5,6]naphtho[1,2- | (m, 2H), 3.21-3.05 (m, 1H), 3.02 (s, 3H), |
| d]azepin-8-yl)cyclobutanecarboxamide | 2.27-1.35 (m, 16H), 1.25 (s, 3H), 1.21-1.05 |
| | (m, 2H), 0.93-0.80 (m, 5H). ESI-MS (m/z) |
| | Calcd for C₂₅H₃₈N₂O₃: 414.2. Found 415.4 |
| | [M + 1]⁺. HPLC purity: 98.15%. |
| methyl (5aR,7aS,8S)-5a,7a-dimethyl-2-oxo- | ¹H-NMR (300 MHz, CDCl₃) δ: 6.06 (brs, 1H), |
| 2,3,4,5,5a,5b,7a,8,9,10,10a,10b,11,12- | 5.73 (s, 1H), 5.31 (s, 1H), 3.66 (s, 3H), |
| tetradecahydrocyclopenta[5,6]naphtho[1,2- | 3.24-3.22 (m, 2H), 2.68-2.62 (m, 3H), 2.45-2.33 |
| d]azepine-8-carboxylate | (m, 3H), 2.22-1.85 (m, 6H), 1.35-1.32 |
| | (m, 2H), 1.25 (s, 3H), 0.58 (s, 3H). ESI-MS |
| | (m/z) Calcd for C₂₁H₂₉NO₃: 343.2. Found |
| | 344.0 [M + 1]⁺. HPLC purity: 95.18%. |
| (5aR,6S,7aS,8S)-6-hydroxy-N-isopropyl- | ¹H-NMR (300 MHz, CDCl₃) δ: 6.09 (brs, 1H), |
| 5a,7a-dimethyl-3-oxo- | 5.61 (s, 1H), 5.27 (s, 1H), 5.15(d, J = |
| 2,3,4,5,5a,5b,6,7,7a,8,9,10,10a,10b,11,12- | 7.8 Hz, 1H), 4.26 (bs, 1H), 4.04-3.97 (m, |
| hexadecahydrocyclopenta[5,6]naphtho[2,1- | 1H), 3.20-3.14 (m, 2H), 2.53-2.47 (m, 1H), |
| c]azepine-8-carboxamide | 2.09-1.90 (m, 8H), 1.32 (s, 3H), 1.11 (d, J = |
| | 6.6 Hz,, 6H), 1.08 (s, 3H),0.87-0.85 (m, 6H). |
| | ESI-MS (m/z) Calcd for C₂₃H₃₆N₂O₃: 388.2. |
| | Found 389.0 [M + 1]⁺. HPLC purity: 90.83%. |

### Example 6: In Vitro PGC-1α Expression Studies

Culture conditions: DMEM containing 25 mM glucose with 10% FBS supplementation; grown at 37° C. and 5% CO₂.

Drugs: (+)-Epicatechin, AICAR (direct activator of AMPK).

Assay Conditions: C2C12 cells were cultured in DMEM supplemented in 10% FBS upto 80% confluence. The cells were further trypsinized and seeded into a 96 well plate at a density of 5000 cells/well (well volume—100 μL) (Day 0). Following attachment, the cells were allowed to reach 80-90% confluence in the plate (typically 48 hours post seeding) and further differentiated using DMEM+2% Horse Serum (200 μL/well). The media was changed every day or at least every alternate day for 5 days to allow myoblasts to differentiate into myotubes. On day 7 (post seeding), the cells were treated with standards and test compounds for PGC-1 α assay.

PGC-1α assay: For the detection of PGC-1a, the cells were incubated with 0.5 μg/ml primary antibody (Merck, WH0010891M3) in PBS-T containing 5% BSA at 4° C. overnight. The cells were then washed three times with PBS-T for 5 minutes and incubated with 1:1000 dilution secondary antibody (Anti-rabbit IgG, HRP-linked Antibody, Cell Signaling) in PBS-T with 1% BSA for 1 hour at RT. Cells were washed three times with PBS-T for 5 minutes The cells were incubated with 100 μl TMB substrate solution for 30 minutes and the reaction was stopped with 100 μl of 2N H₂SO₄. Then the plate was read at 450 nM using ELISA plate reader and absorbance recorded. % activity was calculated using DMSO control as 100%. (For plate optical density calculations, a background correction was taken at 540 nm). The final PGC-1a activation % is average of activation of 6 replicates.

TABLE 3
Effect of Compound 26 on PGC-1α expression in C2C12
cells (OD @ 650 nm) (5000 cells/well)

| | | |
|---|---|---|
| Treatment | Concentartion | % Activity |

| | |
|---|---|
| DMSO | 100 |

| | | | |
|---|---|---|---|
| AICAR | 1 | mM | 148 |
| Epicatechin | 0.001 | nM | 129 |
| Compound 26 | 0.01 | nM | 129 |

TABLE 4
Effect of Compound 26 on PGC-1α expression in C2C12
cells (OD @ 650 nm) (5000 cells/well)

| | | |
|---|---|---|
| Treatment | Concentration | % Activity |

| | |
|---|---|
| DMSO | 100 |

| | | | |
|---|---|---|---|
| AICAR | 1 | mM | 140 |
| Epicatechin | 0.001 | nM | 123 |
| Compound 26 | 0.01 | nM | 146 |

TABLE 5
Effect of Compound 36 on PGC-1α expression in C2C12
cells (OD @ 650 nm) (5000 cells/well)

| | | |
|---|---|---|
| Treatment | Concentration | % Activity |

DMSO

| | | | |
|---|---|---|---|
| AICAR | 1 | mM | 130 |
| Epicatechin | 0.001 | nM | 120 |
| Compound 36 | 0.01 | nM | 133 |

TABLE 6
Effect of Compound 40 on PGC-1α expression in C2C12
cells (OD @ 650 nm) (5000 cells/well)

| | | |
|---|---|---|
| Treatment | Concentration | % Activity |

| | |
|---|---|
| DMSO | 100 |

| | | | |
|---|---|---|---|
| AICAR | 1 | mM | 130 |
| Epicatechin | 0.001 | nM | 122 |
| Compound 40 | 0.01 | nM | 130 |

TABLE 7
Effect of Compound 40 on PGC-1α expression in C2C12
cells (OD @ 650 nm) (5000 cells/well)

| | | |
|---|---|---|
| Treatment | Concentration | % Activity |

| | |
|---|---|
| DMSO | 100 |

| | | | |
|---|---|---|---|
| AICAR | 1 | mM | 140 |
| Epicatechin | 0.001 | nM | 125 |
| Compound 40 | 0.01 | nM | 143 |

TABLE 8
Effect of Compound 40 on PGC-1α expression in C2C12
cells (OD @ 650 nm) (5000 cells/well)

| | | |
|---|---|---|
| Treatment | Concentration | % Activity |

| | | | |
|---|---|---|---|
| AICAR | 1 | mM | 128 |
| Epicatechin | 0.001 | nM | 120 |
| Compound 36 | 0.01 | nM | 130 |

### Example 7: In Vivo PGC-1α Expression Studies

C57BL/6 mice were procured from Vivo Bio Tech Ltd. Male mice were used for the study within an age range of 7 to 8 weeks. Body weight for these mice ranged from 20 g to 25 g. Animals were housed in a group of 6 in individually ventilated cages with temperature of 22±3° C., a relative humidity of 50 to 60%. The bottom of the cages was layered with sterilized corn cob. Animals were provided with gamma irradiated feed and autoclaved drinking water ad-libitum. The IVCs were maintained at an environment controlled (Centrally Air-Conditioned) room at a temperature of 22±3° C., a relative humidity of 50 to 60%, light intensity of 250-300 lux and a 12 hour light-dark cycle and noise intensity of <85 db.

Method of Preparation: LPS was dosed at 3 mg/kg in a dose volume of 10 ml/kg. For this, 0.3 mg/ml of LPS solution (Micronized suspension) was prepared. Butter paper was placed on analytical balance, tarred and LPS was placed on butter paper and its weight was noted down. LPS was transferred to 15 ml falcon tube. Then required amount of normal saline was added to it to make it 0.3 mg/ml. The solution was vortexed properly for 5 minutes and placed on ice. LPS solution was vortexed every time before aspiration and injection.

LPS was dosed at 3 mg/kg. Each test compound was dosed at 10 mg/kg, 2 hour and 15 min post LPS administration.

0.5% CMC was used as a vehicle with a dose volume of 10 ml/kg. An IP route was used for LPS, and a PO route was used for each test compound. Animals were sacrified 24 hours post LPS dosing, and plasma were collected. PGC-1α level was measured in liver samples, and other tissues were stored for future analysis. PGC-1α level was checked using ELISA kit—CUSABIO Mouse PPARGC1A ELISA kit.

Protocol: One day before start of the experiment C57BL/6 Mice (Male, 6-7-week-old) were randomized on the basis of their body weight and were divided into groups. All the cages were assigned the respective group name. Animals inside each cage were marked on the base of the tails with permanent marker to distinguish each animal. Mice were fasted overnight. Next day morning animals were dosed with test item. Group 1 (Control) and Group 2 (LPS) Animals were dosed with vehicle (0.5% CMC). Test items were prepared in 0.5% CMC by trituration in mortar and pestle and addition of CMC drop by drop. 2 hour post test item/vehicle dosing animals were administered with LPS through IP route at 3 mg/kg of body weight. Group 1 (Control) animals were administered with saline. 0.25 hour post LPS administration, animals were again dosed with test item/vehicle. Post dosing, animals were returned to their respective cages and kept until termination of the study. No feed was offered to any group. Animals were provided with water. 24 hour post LPS administration, blood was collected from each animal under influence of mild isoflurane anesthesia through retro-orbital technique. Animals were sacrificed by CO₂ asphyxiation technique. Post-termination, animals were dissected and vital organs, liver, brain, skeleton muscle were collected in Eppendorf tubes and flash frozen in liquid nitrogen. Serum was collected from whole blood by centrifuging it at 4° C. at 10,000 RPM and for 10 minutes. (Blood was left for 2 hour for coagulation before centrifugation) After completion of the experiment, all collected samples were stored at −80° C. until analysis.

Assay Methods/Protocol: The mortar was chilled with liquid nitrogen and then the small tissue pieces were grinded in the presence of liquid nitrogen to a fine powder. Immediately after grinding, transferred small amount of tissue powder to a 1.5 ml micro centrifuge tube containing 1.0 ml lysis buffer (RIPA buffer+Protease inhibitor). The samples were then incubated on ice for 2 hours. The samples were vortexed every 15 minutes. The samples were then centrifuged at 13000×g for 15 minutes at 2-8° C. After centrifugation the supernatant was removed carefully and pellet was discarded. The protein assay was performed using a Bio-Rad Protein Assay Dye Reagent Concentrate (#5000006).

BSA standard curve and protein estimation: The Bio-Rad dye reagent was prepared by diluting 1 part dye reagent with 4 parts of distilled water. The dilutions for BSA were prepared from 10 mg/ml stock. The range of the dilutions was 1.0 mg/ml to 0.0125 mg/ml. For protein estimation 10 μl of each standard or sample was added to 96 well plate. To this 200 μl of diluted dye reagent was added. The samples and reagent were mixed thoroughly using micro plate mixer. The samples were incubated at room temperature for 5 minutes. The plate was read at 595 nm using Spectramax Me5.

For PGC-op assay ELISA, samples were diluted in such a way that the saturation in signal can be avoided. This was achieved by running a small experiment every time with 8 wells. The 3-4 dilutions concentrations of a sample with known protein concentration were prepared and PGC-1α assay was ran along with 3-4 standard dilutions with lowest and highest value (pg/ml). This was to decide on which dilution should be used for final assay. After the confirmation of the working dilution, all the samples were diluted accordingly, and PGC-1α assay was performed according to the kit protocol. The final values were multiplied by the dilution factor. The data was analyzed using the PGC-1α standard curve.

TABLE 9
in vivo PGC-1α expression studies.

| | PGC-1α levels | PGC-1α level |
|---|---|---|
| Groups | (pg/ml) | (%) |
| Control | 38327 | 100 |
| LPS (3 mg/Kg) | 16860 | 44 |
| LPS (3 mg/Kg) + Compound 36 | 29341 | 77 |
| (BID) | | |
| LPS (3 mg/Kg) + Compound 26 | 32298 | 84 |
| (BID) | | |
| | PGC-1α level | PGC-1α level |
| Groups | (pg/ml) | (%) |
| Control | 53181 | 100 |
| LPS (3 mg/Kg) | 27051 | 51 |
| LPS (3 mg/Kg) + Epicatechin | 33330 | 63 |
| (BID) | | |
| LPS (3 mg/Kg) + Compound 40 | 39014 | 73 |
| (BID) | | |
| Control | 35303 | 100 |
| LPS (3 mg/Kg) | 18139 | 51 |
| LPS (3 mg/Kg) + Epicatechin | 31729 | 90 |
| (BID) | | |
| LPS (3 mg/Kg) + Compound 50 | 38684 | 110 |
| (BID) | | |
| Control | 30334 | 100 |
| LPS (3 mg/Kg) | 15099 | 50 |
| LPS (3 mg/Kg) + Epicatechin | 21967 | 72 |
| (BID) | | |
| LPS (3 mg/Kg) + Compound 40 | 22668 | 75 |
| (BID) | | |
| LPS (3 mg/Kg) + Compound 50 | 24206 | 80 |
| (BID) | | |
| Control | 6620 | 100 |
| LPS-0.25 + 0.5 + 1 mg/kg | 2654 | 40 |
| LPS-0.25 + 0.5 + 1 mg/kg, | 3247 | 49 |
| Epicatechin −10 mg/kg | | |
| LPS-0.25 +0.5 + 1 mg/kg, | 2632 | 40 |
| Compound 50 −10 mg/kg | | |

### Example 8: Pharmacokinetics of Compounds

C57BL/6 Mice, Age 7-9 weeks, body weight range 20-25 g were used. The mice were fasted overnight. Mice were dosed once a day for a single day at 10 mg/kg, 10 mg/kg. 6 time-points were used for whole blood. 10% NMP was used as a vehicle. 9 animals were used per treatment, with three groups of three animals each. Group 1 had time points at 15 and 60 minutes, Group 2 had time points at 30 and 120 minutes, and Group 3 had time points at 90 and 240 minutes. An Oral (PO) route was used. Plasma and brain samples were collected (brain sample collected at terminal time point). Plasma was collected through the centrifugation of whole blood at 10,000 rpm for 10 minutes at 4° C. Samples were stored at −80° C. until processing.

### LCMS-Plasma Method:

1.00 mg/mL in 100% MeOH PP vials (prepared at RT yellow light) at 2-8° C. were used as stock solution. 12.22-50000 ng/mL using 80% MeOH PP vials (prepared at RT yellow light) at 2-8° C. were used as working solution. 0.98-4000 ng/mL (prepared at RT yellow light) was used for the curve range. For Compounds 26, 40, and 50, 1.00 mg/mL of Compound 53 in 100% methanol Ria vials (Prepared at RT yellow light) was used as IS stock solution, and 80 ng/mL of Compound 53 in 80% methanol (Prepared at RT yellow light) was used as IS working solution. For Compound 36, 1.00 mg/mL of Compound 47 in 100% methanol Ria vials (Prepared at RT yellow light) was used as IS stock solution, and 80 ng/mL of Compound 47 in 80% methanol (Prepared at RT yellow light) was used as IS working solution. A linear, 1/×2 fit type was used. The injection volume was 10 μL for Compounds 26 and 36, and 5 μL for Compounds 40 and 50. Mobile Phase A was 0.1% Formic Acid in water at room temperature. Mobile Phase B was 100% methanol at room temperature. R0 wash solution was 80% methanol at room temperature. R3 wash solution was IPA/MeOH/ACN/water (1:1:1:1) at room temperature. The column was Agilent Eclipse 50*2.1 mm 1.8 μm and the instrument was Shimadzu LC-40 UPLC and AB Sciez 6500+ with LCMS002.

Using a pipette, 25 μL of each sample were aliquoted (calibration curve sample, QC samples and blank matrix) to labeled 1.5 ml eppendorf. Using a pipette, 5.0 μL of IS of conc. 80 ng (Cocktail IS) was aliquoted to each standards and samples except Blank. All tubes were mixed on a multi-tube vortex for approximately 30 seconds. 50 μL of 10 mM Sodium bi-carbonate were added. 1 ml of tert-butyl methyl ether (TBME) was added. All tubes were mixed on a multi-tube vortex for approximately 5 minutes then centrifuged for 10 min at 5,000 rpm for Compounds 26 and 36 and at 10,000 rpm for Compounds 40 and 50, 4° C. The sample was kept in methanol containing dry ice for few seconds to freeze the Aqueous layer (flash freeze method). The upper organic layer was poured from the frozen tubes into the labeled 12×75-mm rial vials and evaporated to dryness under nitrogen using Liquid Evaporator (30° C., ˜15 psi, ˜10 mins). 200 μl of reconstitution solution (0.1% Formic Acid: MeOH (20:80 v/v) were added into each tube. Samples were Vortexed for 30 seconds then transferred to amber glass injection vials for injection.

After test item administration, blood samples were collected from individual animals in previously labeled Heparinised blood collection tubes. Blood samples were collected from retro orbital plexus. The time points for collection of blood samples from all the animals are; 0.25, 0.5, 1, 2 and 4 hr. The collected samples were subjected to centrifugation @ 10,000 rpm for 10 minutes at 40° C. for separation of plasma. Thereafter, plasma samples were collected in previously labeled plasma tubes. These were stored at −80° C. until analysis.

### LCMS-Brain Method:

1.00 mg/mL in 100% MeOH PP vials (prepared at RT yellow light) at 2-8° C. were used as stock solution. 12.22-50000 ng/mL using 80% MeOH PP vials (prepared at RT yellow light) at 2-8° C. were used as working solution. 0.48-2000 ng/mL (prepared at RT yellow light) was used for the curve range. For Compounds 26, 40, and 50, 1.00 mg/mL of Compound 53 in 100% methanol Ria vials (Prepared at RT yellow light) was used as IS stock solution, and 80 ng/mL of Compound 53 in 80% methanol (Prepared at RT yellow light) was used as IS working solution. For Compound 36, 1.00 mg/mL of Compound 47 in 100% methanol Ria vials (Prepared at RT yellow light) was used as IS stock solution, and 80 ng/mL of Compound 47 in 80% methanol (Prepared at RT yellow light) was used as IS working solution. A linear, 1/×2 fit type was used. The injection volume was 10 μL for Compounds 26 and 36, and 5 μL for Compounds 40 and 50. Mobile Phase A was 0.1% Formic Acid in water at room temperature. Mobile Phase B was 100% methanol at room temperature. R0 wash solution was 80% methanol at room temperature. R3 wash solution was IPA/MeOH/ACN/water (1:1:1:1) at room temperature. The column was Agilent Eclipse 50*2.1 mm 1.8 μm and the instrument was Shimadzu LC-40 UPLC and AB Sciez 6500+ with LCMS002.

100 mg of brain tissue were weighed, 400 μL of Ice cold PBS was added, and samples were homogenated using polytron homogenizer. Before starting the experiment the blade was cleaned with NS, Methanol, Methanol: Water (50:50) followed by NS. Using a pipette with Methanol-rinsed pipette tips, homogenate 50 μL of each sample (calibration curve sample, QC samples and blank matrix) was aliquoted to labeled 1.5 ml Ependorfs. Using a pipette, 5.0 μL of IS of conc. 80 ng (Cocktail IS) was aliquoted to each standards and samples except Blank. All tubes were mixed on a multi-tube vortex for approximately 30 seconds. 1 ml of tert-butyl methyl ether (TBME) was added. All tubes were mixed on a multi-tube vortex for approximately 5 minutes then centrifuged for 10 min at 5,000 rpm, 4° C. The sample was kept in methanol containing dry ice for few seconds to freeze the Aqueous layer (flash freeze method). The upper organic layer was poured from the frozen tubes into the labeled 12×75-mm rial vials and evaporated to dryness under nitrogen using Liquid Evaporator (30° C., ˜15 psi, ˜10 mins). 200 μl of reconstitution solution (0.1% Formic Acid: MeOH (20:80 v/v) were added into each tube. The samples were vortexed for 30 seconds then transferred to amber glass injection vials for injection.

At the terminal time points, animals were euthanized after blood sample collection. Their skulls were opened and whole brains were separated and collected in previously labeled tubes. The tubes were then immediately transferred into liquid nitrogen container for snap freezing. Later the samples were stored at −80° C.

TABLE 10
Quantitative Determination of Compounds in Mice Plasma.
Plasma

| | T½ | Tmax | Cmax | AUClast | AUCINF |
|---|---|---|---|---|---|
| Compound | 8 hr) | (hr) | (ng/mL) | (ng*hr/mL) | (ng*hr/mL) |
| Compound 26 | 1.033 | 0.25 | 2134.4 | 930.5 | 992.9 |
| Compound 36 | 1.110 | 0.25 | 1956.3 | 1498.0 | 1639.6 |
| Compound 40 | 0.933 | 0.25 | 2200.2 | 2298.6 | 2434.0 |
| Compound 50 | 0.424 | 0.25 | 2898.7 | 2446.7 | 2535.0 |

TABLE 11
Quantitative Determination of Compounds in Mice Brains.

| | Tmax | Cmax | AUClast |
|---|---|---|---|
| Compound | (hr) | (ng/ml) | (ng*hr/mL) |
| Compound 26 | 1.0 | 432.8 | 486.6 |
| Compound 36 | 1.0 | 1164.3 | 1636.1 |
| Compound 40 | 1.0 | 1682.7 | 2503.6 |

### Example 9: Hydrolysis Conditions

To assess whether the fraction of synthetic and reverse hydrolytic activities of mitochondrial ATP synthase can be measured in the same preparation, a protocol was developed to monitor changes in oxygen consumption and proton release in response to changes in mitochondrial ATP synthase activity. The proton released and the concomitant changes in the pH can be monitored using the ECAR (extracellular acidification) channel in the Agilent Seahorse XF analyzer using isolated mitochondria, to ensure that the changes in pH cannot be attributed to glycolysis or other contributors to ECAR.

Mice were anesthetized with isoflurane followed by a cervical dislocation, and the heart was immediately removed and placed in ice cold relaxation buffer (5 mM sodium pyrophosphate, 100 mM KCl, 5 mM EGTA, 5 mM HEPES; pH 7.4). The heart was squeezed with tweezers to remove blood, minced with scissors, and then placed in a glass-glass Dounce homogenizer with 3 mL of HES homogenization buffer (250 mM sucrose, 5 mM HEPES, 1 mM EDTA; pH to 7.2, adjusted with KOH). The heart tissue was homogenized first with the loose pestle and followed by the tight pestle. The homogenized tissue was placed in a pre-chilled 15 mL conical tube and centrifuged at 900×g (4° C.) for 10 minutes (min). The supernatant was removed, placed in a new tube and centrifuged again at 900×g for 10 min. The supernatant was then transferred to 2 mL microcentrifuge tubes and centrifuged at 10,000×g (4° C.) for 10 min. The mitochondrial pellets were re-suspended in ice cold HES buffer and mitochondrial protein was measured with a BCA assay (Pierce). The concentrated mitochondrial pellet was stored on ice.

Heart mitochondria (0.75-1.5 μg) were loaded into a Seahorse XF96 microplate in L of MAS (70 mM Sucrose, 220 mM Mannitol, 5 mM KH2PO4, 5 mM MgCl2, 1 mM EGTA, 2 mM HEPES; pH 7.2) plus 1% free fatty acid BSA containing substrates. The loaded plate was centrifuged at 2,000×g for 5 min at 4° C. (no brake) and an additional 130 μL of MAS was added to each well. When assessing compounds effect on respirometry, the compounds were added at this point at the indicated concentration in MAS buffer. To avoid disrupting mitochondrial adherence to the bottom of the plate, MAS was added using a multichannel pipette pointed at a 450 angle to the top of the well-chamber, as instructed by the manufacturer. Substrate concentrations in the well when assay was starting in state 4 were as follow: (i) 5 mM pyruvate+5 mM malate or ii) 5 mM succinate+2 μM rotenone. Substrate concentrations in the well when assay was starting in state 3 were as follow: (i) 5 mM pyruvate+5 mM malate+4 mM ADP or ii) 5 mM succinate+2 μM rotenone+4 mM ADP. Injections were performed as indicated in the figure descriptions at the following final concentration in the well: oligomycin (3.5 μM), FCCP (4 μM), Antimycin A (2 μM). Compounds were added at the indicated concentration.

ATP hydrolysis capacity or state 4 acidification was measured using Seahorse XF96 as described in Divakaruni et al. (2018) Anal Biochem 552:60-65 and Acin-Perez et al. (2021) Life 11(9):949, in MAS (70 mM Sucrose, 220 mM Mannitol, 5 mM KH₂PO₄, 5 mM MgCl₂, 1 mM EGTA, 2 mM HEPES; pH 7.2). Plates were loaded with 0.75-1.5 μg of mouse heart mitochondria, or 25 μg of cell lysate. Cell lysates were prepared by subjecting the samples to 4 cycles of free thaw (liquid nitrogen—37° C. water bath) before measuring protein concentration. When assessing compounds effect on respirometry, the compounds were added in MAS after sample centrifugation. Initial respiration of the samples was sustained by the addition of 5 mM succinate+2 μM rotenone in the MAS after centrifugation. Injections were performed at the following final concentration in the well: Antimycin A (2 μM), oligomycin (5 μM), FCCP (1 μM), ATP (20 mM). To assess maximal ATP concentration, ATP was injected consecutively.

Oxygen consumption was measured in isolated fresh intact mitochondria from mouse heart, as oxygen consumption is linked to maximal ATP synthesis and proton release when mitochondria are respiring either in the presence of substrates and ADP (ATP synthesizing mitochondria or state 3) or in the presence of substrates but not ADP (state 4). Oxygen consumption was fueled by Pyruvate plus Malate (Pyr+Mal) or Succinate plus Rotenone (Succ+Rot). Proton release in isolated mitochondria is mostly a result of ATP hydrolysis.

An additional assay was developed to determine the maximal ATP hydrolysis capacity in frozen mitochondria. Maximal ATP hydrolytic capacity was assessed in frozen heart mitochondria where mitochondrial respiration is inhibited by Antimycin A and maximal ATP hydrolysis is driven by co-injection of FCCP and ATP, followed by inhibition in the presence of oligomycin. Addition of oligomycin at different concentrations in the assay media revealed a dose dependent inhibition of ATP hydrolysis, demonstrating the specificity of the assay.

OCR and ECAR were measured in a Seahorse XF96 analyzer under basal conditions as well as after injection of 2 μM oligomycin, two sequential additions of 1.5 μM FCCP, followed by 1 μM rotenone with 2 μM antimycin A. Respiratory parameters were calculated according to standard protocols, and all rates were corrected for non-mitochondrial respiration/background signal by subtracting the oxygen consumption rate insensitive to rotenone plus antimycin A.

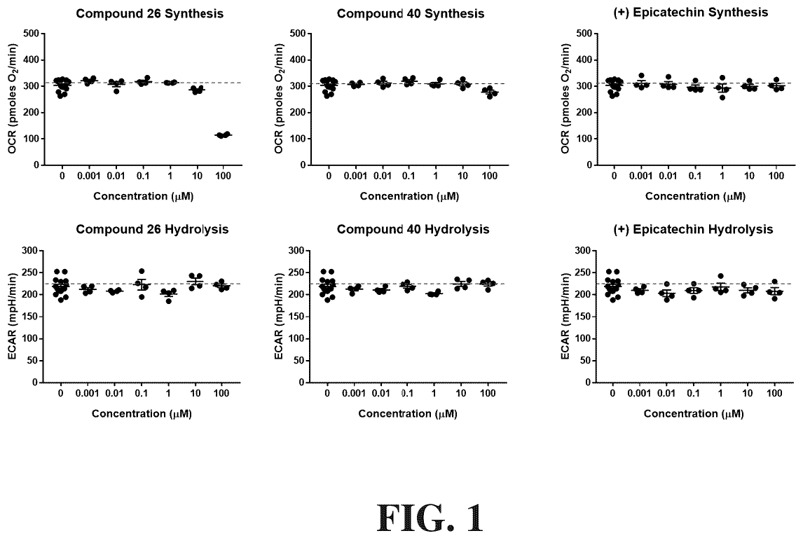

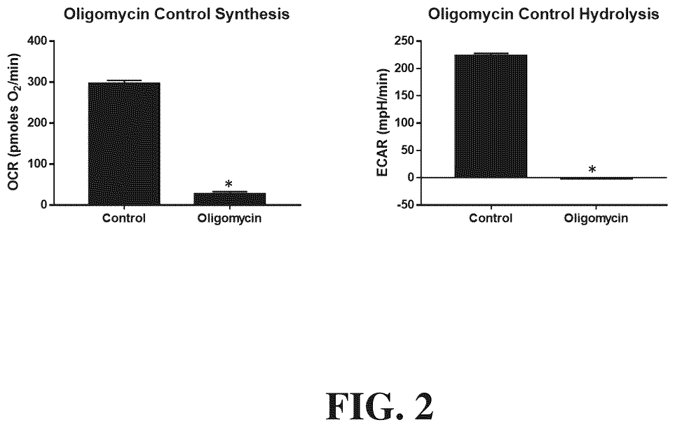

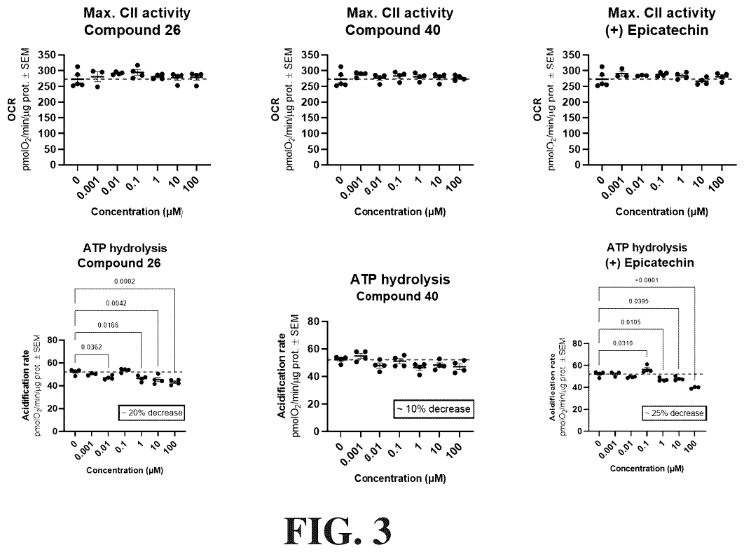

Compound 26 and Compound 40 were tested. Compound 26 decreased ATP synthesis at the highest concentration and may be a Complex I inhibitor (FIG. 1). For epicatechin, there was no change in ATP synthesis. Oligomycin controls worked as expected for both ATP synthesis and hydrolysis (FIG. 2), but only mild changes in hydrolysis were observed for Compounds 26 and 40 in this assay. Therefore, frozen mitochondria were used to robustly test hydrolysis. Epicatechin showed no synthesis change with Complex I or Complex II substrates and showed a 10-25% decrease in hydrolysis (FIG. 3).

All documents, including patents, patent application and publications cited herein, including all documents cited therein, tables, and drawings, are hereby expressly incorporated by reference in their entirety for all purposes.

While the foregoing written description of the compounds, uses, and methods described herein enables one of ordinary skill in the art to make and use the compounds, uses, and methods described herein, those of ordinary skill in the art will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

1. A compound of Formula (I): 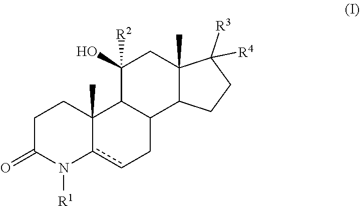
or a pharmaceutically acceptable salt, stereoisomer or tautomer thereof, wherein:
R¹ is H or C₁₋₆ alkyl;
R² is H, D, or C₁₋₆ alkyl;
R³ is C₁₋₆ alkyl, C₁₋₆ alkoxy, C(O)R⁵, NC(O)R⁵, NC(O)NR₆R₇, C(O)OR⁵, NHR⁵, or 5-membered heterocyclyl, wherein C₁₋₆ alkyl is optionally substituted with one or more of OH or D, and 5-membered heterocyclyl is optionally substituted with C₁₋₆ alkyl;
R⁴ is H;
or R³ and R⁴ taken together with the carbon atom to which they are attached form a 5-membered heterocycle;
R⁵ is H, C₁₋₆ alkyl, C₃₋₁₀ cycloalkyl, SO₂R⁶, NR⁶R⁷ or 5-6 membered heterocyclyl optionally substituted with one or more C₁₋₆ alkyl;
R⁶ and R⁷ are each independently H, C₃₋₆ cycloalkyl, or C₁₋₆ alkyl optionally substituted with C₃₋₆ cycloalkyl; and
the dashed line represents an optional double bond.

2. The compound of claim 1, wherein the compound is of Formula (Ia):

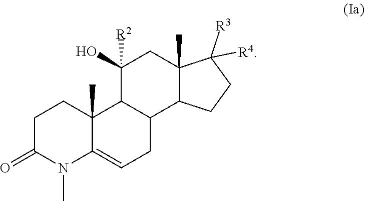

3. The compound of claim 2, wherein R² is H.

4. The compound of claim 2 or 3, wherein R⁴ is H.

5. The compound of claim 1, wherein the compound is of Formula (Ib):

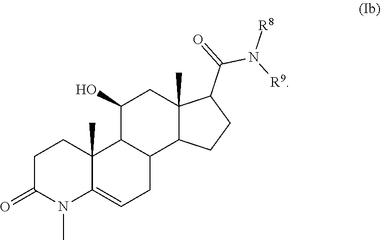

6. The compound of claim 5, wherein R⁹ is H.

7. The compound of claim 1, wherein the compound is of Formula (Ic):

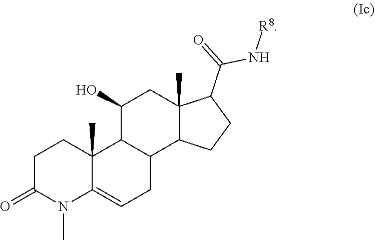

8. The compound of claim 7, wherein R⁸ is C₁₋₆ alkyl.

9. The compound of claim 8, wherein R⁸ is methyl.

10. The compound of claim 8, wherein R⁸ is isohexyl.

11. The compound of claim 7, wherein R⁸ is C₃₋₁₀ cycloalkyl.

12. The compound of claim 7, wherein R⁸ is C₁₋₆ alkyl substituted with C₃₋₁₀ cycloalkyl.

13. The compound of claim 1, wherein the compound is of Formula (Id):

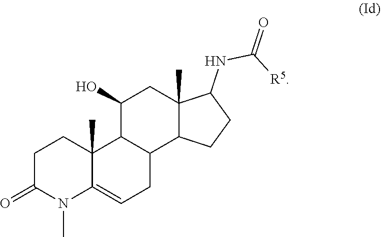

14. The compound of claim 13, wherein R⁸ is C₃₋₁₀ cycloalkyl.

15. The compound of claim 14, wherein R⁵ is cyclopropyl.

16. The compound of claim 13, wherein the compound is of Formula (Ie):

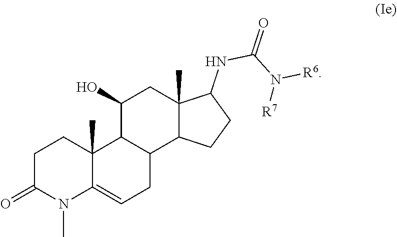

17. The compound of claim 16, wherein R⁶ is C₃₋₁₀ cycloalkyl or C₁₋₆ alkyl optionally substituted with C₃₋₁₀ cycloalkyl.

18. The compound of claim 16 or 17, wherein R⁷ is H.

19. A compound of Formula (II): 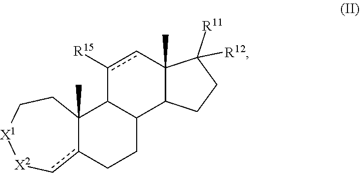
or a pharmaceutically acceptable salt, stereoisomer or tautomer thereof, wherein:
one of X¹ and X² is NR¹⁰, and the other is C═O,
R¹⁰ is H or C₁₋₆ alkyl;
R¹¹ is C₁₋₆ alkoxy, C(O)OR¹³, C(O)NR¹³R¹⁴, C(O)R¹³, NR¹³R¹⁴, NC(O)R¹³, or C₁₋₆ alkyl optionally substituted with one or more R¹³;
R¹² is H or OH; or
R¹¹ and R¹² together form oxo;
R¹³ and R¹⁴ are each independently H, C₁₋₆ alkyl, C₃₋₆ cycloalkyl, or NH₂;
R¹⁵ is H or OH; and
each dashed line represents an optional double bond.

20. The compound of claim 19, wherein R¹⁵ is OH.

21. The compound of claim 19, wherein the compound is of Formula (IIa):

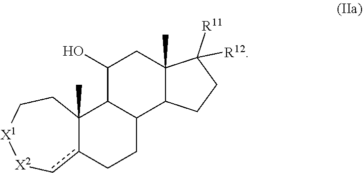

22. The compound of claim 21, wherein R¹² is H.

23. The compound of claim 21 or 22, wherein X¹ is NR¹⁰ and X² is C═O.

24. The compound of claim 21, wherein the compound is of Formula (IIb):

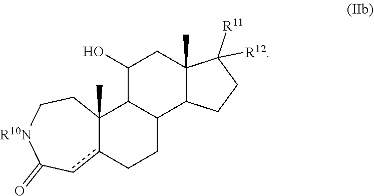

25. The compound of claim 24, wherein R¹⁰ is C₁₋₆ alkyl.

26. The compound of claim 25, wherein R¹⁰ is methyl.

27. The compound of claim 24, wherein the compound is of Formula (IIc):

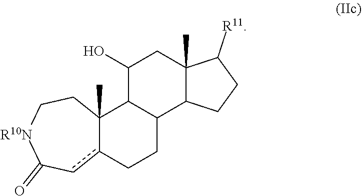

28. The compound of claim 27, wherein R¹⁰ is methyl.

29. The compound of claim 27 or 28, wherein R¹¹ is C₁₋₆ alkoxy.

30. The compound of claim 27 or 28, wherein R¹¹ is NH₂.

31. The compound of claim 27, wherein the compound is of Formula (IId):

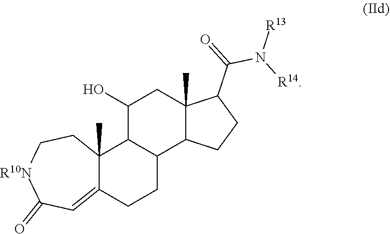

32. The compound of claim 31, wherein R¹⁰ is methyl.

33. The compound of claim 31 or 32, wherein R¹³ and R¹⁴ are each independently C₁₋₆ alkyl.

34. The compound of claim 33, wherein R¹³ and R¹⁴ are each methyl.

35. The compound of claim 27, wherein the compound is of Formula (IIe):

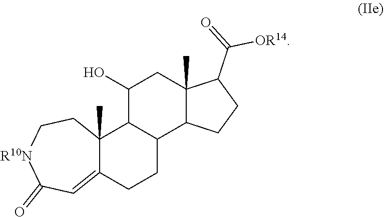

36. The compound of claim 35, wherein R¹⁰ is methyl.

37. The compound of claim 35 or 36, wherein R¹⁴ is hydrogen or methyl.

38. The compound of claim 24, wherein the compound is of Formula (IIg):

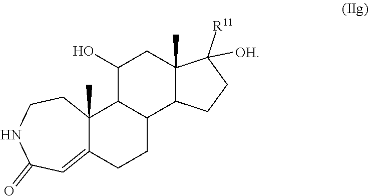

39. The compound of claim 38, wherein R¹¹ is C(O)NHR¹³, wherein R¹³ is C₁₋₆ alkyl or C₃₋₆ cycloalkyl.

40. The compound of claim 21, wherein the compound is of Formula (IIh):

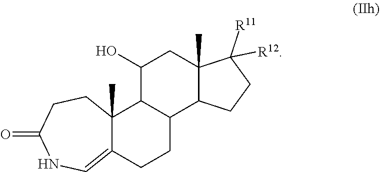

41. The compound of claim 40, wherein R¹¹ is C(O)NHR¹³, wherein R¹³ is C₁₋₆ alkyl or C₃₋₆ cycloalkyl.

42. The compound of claim 41, wherein R¹³ is isopropyl or cyclobutyl.

43. A compound of Formula (III): 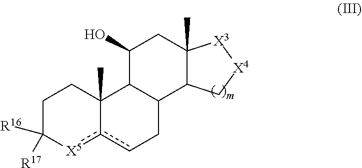
wherein
R¹⁶ and R¹⁷ together form oxo, or
R¹⁶ and R¹⁷ together with the carbon atom to which they are attached form a 5-membered heterocycle;
one of X³ and X⁴ is CH₂ or C═O, and the other is NR¹⁸;
X⁵ is CH₂, CH, or NR¹⁹;
R¹⁸ is H, C₁₋₆ alkyl, or C(O)R¹⁹;
each R¹⁹ is independently H or C₁₋₆ alkyl;
m is 1 or 2; and
the dashed lines represent optional double bonds, wherein the number of dashed lines representing double bonds is zero or one.

44. The compound of claim 43, wherein the compound is of Formula (IIIa):

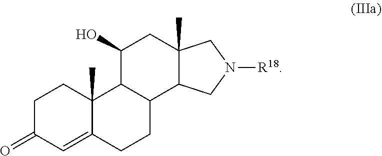

45. The compound of claim 43, wherein the compound is of Formula (IIIb):

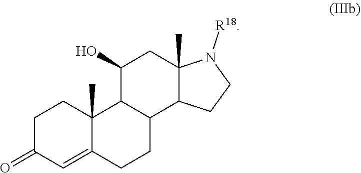

46. The compound of claim 43, wherein the compound is of Formula (IIIc):

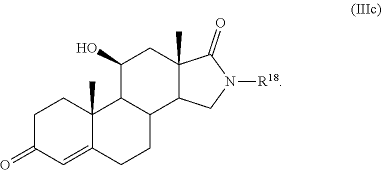

47. The compound of claim 43, wherein the compound is of Formula (IIId):

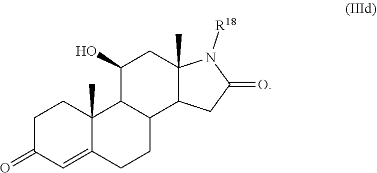

48. The compound of claim 43, wherein the compound is of Formula (IIIe):

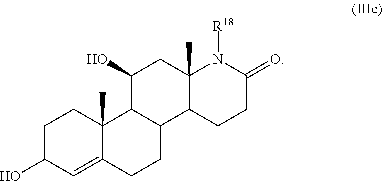

49. A compound selected from the group consisting of: [symbol]
or a pharmaceutically acceptable salt, stereoisomer or tautomer thereof.

50. A compound selected from the group consisting of: [symbol]
or a pharmaceutically acceptable salt, stereoisomer or tautomer thereof.

51. A compound selected from the group consisting of [symbol]
or a pharmaceutically acceptable salt, isomer, or tautomer thereof.

52. A compound selected from the group consisting of [symbol]
or a pharmaceutically acceptable salt, isomer, or tautomer thereof.

53. A pharmaceutical composition comprising a compound of any one of claims 1-52, a pharmaceutically acceptable salt thereof, and a pharmaceutically acceptable carrier.

54. A compound, or a pharmaceutically acceptable salt thereof, of any one of claims 1-52 for use as therapeutically active substance.

55. Use of a compound, or a pharmaceutically acceptable salt thereof, of any one of claims 1-52 or a pharmaceutical composition of claim 53 for the preparation of a medicament for the treatment of a disease, disorder or condition.
